(19) United States
(12) Patent Application Publication
(10) Pub. No.: US 2023/0183664 A1
(43) Pub. Date: Jun. 15, 2023

(54) SELF-LIMITING VIRAL VECTORS ENCODING NUCLEASES

(71) Applicant: Precision BioSciences, Inc., Durham, NC (US)

(72) Inventors: Hui Li, Apex, NC (US); James Jefferson Smith, Morrisville, NC (US); Derek Jantz, Durham, NC (US)

(73) Assignee: Precision BioSciences, Inc., Durham, NC (US)

(21) Appl. No.: 17/998,419

(22) PCT Filed: May 10, 2021
(86) PCT No.: PCT/US2021/031511

§ 371 Date: Nov. 10, 2022

Related U.S. Application Data
(60) Provisional application No. 63/023,165, filed on May 11, 2020.

(51) Int. Cl. C12N 9/22; C12N 15/86

(52) U.S. Cl. CPC C12N 9/22; C12N 15/86; C12N 2830/008; C12N 2830/42; C12N 2750/14143

(57) ABSTRACT

Disclosed herein are viral vectors for use in recombinant molecular biology techniques. In particular, the present disclosure relates to self-limiting viral vectors containing nucleic acid sequences that encode engineered nucleases as well as nuclease recognition sequences such that expression of the engineered nuclease in a cell cleaves the viral vector and limits its persistence time. In some embodiments, the viral vectors disclosed herein also carry directives to delete, insert, or change a target sequence.

### FIELD OF THE INVENTION

The invention relates to the field of molecular biology and recombinant nucleic acid technology. In particular, the invention relates to self-limiting viral vectors comprising genes encoding site-specific endonucleases as well as recognition sequences for site-specific endonucleases such that expression of the endonuclease in a cell cleaves the viral vector and limits its persistence time. Such viral vectors may also carry directives to delete, insert, or change a target sequence. Moreover, the self-limiting viral vectors may be engineered to address kinetic balancing (i.e., ensuring adequate expression of the endonuclease before that endonuclease finds its recognition sequence within the viral vector).

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 10, 2021 is named P89339_1130WO_ST25_5-10-21, and is 33.0 kilobytes in size.

### BACKGROUND OF THE INVENTION

Adeno-associated virus (AAV) is a small virus, which infects humans and several other primate species. AAV is not known to cause disease, and generally causes only a mild immune response. The virus infects both dividing and quiescent cells and can be engineered to persist in an extrachromosomal state without integrating into the genome of the host cell (Russel D W, Deyle D R (2010) Current Opinion in Molecular Therapy. 11: 442-447; Grieger J C, Samulski R J (2005) Advances in Biochemical Engineering/Biotechnology 99: 119-45P). These features make AAV a very attractive candidate for creating viral vectors for gene therapy. Recent human clinical trials using AAV for gene therapy in the retina have shown promise (Maguire A M, et al. (2008) New England Journal of Medicine 358: 2240-8). Moreover, AAV presents a well-known system with an established safety record with the completion of over sixty clinical trials. (Mitchell A M, Nicolson S C, Warischalk J K, and Samulski R J (2010) Curr Gene Ther. 10(5): 319-40).

Wild-type AAV has the ability to stably integrate into the host cell genome at a specific site (designated AAVS1) in the human chromosome 19. This feature makes it somewhat more predictable than other viral vectors such as retroviruses, which present the threat of random insertion and of mutagenesis. Gene therapy vectors based on AAV, however, generally eliminate this integrative capacity by removal of the rep and cap genes from the DNA of the vector. In their place, a gene of interest can be cloned under the control of a promoter between the viral inverted terminal repeats (ITRs) that aid in concatamer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency.

AAV presents disadvantages as well. The cloning capacity of the vector is relatively limited and most therapeutic genes require the complete replacement of the virus's 4.7 kilobase genome. Large genes are, therefore, not suitable for use in a standard AAV vector. Options are currently being explored to overcome the limited coding capacity. The AAV ITRs of two genomes can anneal to form head to tail concatemers, almost doubling the capacity of the vector. Insertion of splice sites allows for the removal of the ITRs from the transcript, alleviating concatemer formation.

Because of AAV's specialized gene therapy advantages, researchers have created an altered version of AAV termed self-complementary adeno-associated virus (scAAV). Whereas AAV packages a single strand of DNA, and must wait for its second strand to be synthesized, scAAV packages two shorter strands that are complementary to each other. By avoiding second-strand synthesis, scAAV can express more quickly, but although as a caveat, scAAV can only encode half of the already limited capacity of AAV (McCarty D M, Monahan P E, Samulski R J (2001) Gene Therapy 8: 1248-54).

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry (Carter, B J (2000) In DD Lassic & N Smyth Templeton. Gene Therapy: Therapeutic Mechanisms and Strategies. New York City: Marcel Dekker, Inc. pp. 41-59).

The Inverted Terminal Repeat (ITR) sequences comprise approximately 145 bases each. The first 125 nucleotides of the ITR sequence are palindromic, folding in on itself to create a T-shaped hairpin structure (Daya, Shyam (2008) Clin. Microbiol. Rev. 21(4) 583-593). The other 20 bases of the ITR remain unpaired and are known as the D sequence. The origin of replication is the ITR and serves as a primer for second-strand synthesis.

With regard to gene therapy, ITRs seem to be the only sequences required in cis next to the therapeutic gene: structural (cap) and packaging (rep) proteins can be delivered in trans. With this assumption, many methods have been established for the efficient production of recombinant AAV (rAAV) vectors containing a reporter, or therapeutic gene. However, it was also published that the ITRs are not the only elements required in cis for effective replication and encapsidation. Some research groups have identified a sequence designated cis-acting Rep-dependent element (CARE) inside the coding sequence of the rep gene. CARE was shown to augment amplification, when present in cis (Nony P, Tessier J, Chadeuf G, et al. (2001) J Virol 75: 9991-4).

On the “left side” of the genome, the rep genes are transcribed from two promoters, p5 and p19, from which two overlapping messenger ribonucleic acids (mRNAs) of different length can be produced. Each of these contains an intron, which may or may not be spliced out. Given these possibilities generated by such a system, four various mRNAs, and consequently, four various Rep proteins with overlapping sequence can be synthesized. Their names depict their sizes in kilodaltons (kDa): Rep78, Rep68, Rep52 and Rep40 (Kyöstiö S R, et al. (1994) Journal of Virology 68: 2947-57). Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins bind ATP and possess helicase activity. As demonstrated, Rep proteins upregulate the transcription from the p40 promoter (mentioned below), but downregulate both p5 and p19 promoters.

The “right side” of a positive-sensed AAV genome encodes overlapping sequences of three capsid proteins, VP1, VP2 and VP3, which start from one promoter, designated p40. The molecular weights of these proteins are 87, 72 and 62 kiloDaltons, respectively. All three are translated from one mRNA, the unspliced transcript producing VP1. After this mRNA is synthesized, it can be spliced in two different manners: either a longer or shorter intron can be excised resulting in the formation of two pools of mRNAs: a 2.3 kb- and a 2.6 kb-long mRNA pool. Generally, especially in the presence of adenovirus, the longer intron is preferred, so the 2.3-kb-long mRNA represents the so-called “major splice.” In this form, the first AUG codon that initiates synthesis of VP1 protein is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine, and serving as the initiation codon for VP2) surrounded by an optimal Kozak context. This contributes to a low level of synthesis of VP2 protein, which is actually VP3 protein with additional N terminal residues, as is VP1. Since the bigger intron is preferred to be spliced out, and since in the major splice the ACG codon is a much weaker translation initiation signal, the ratio at which the AAV structural proteins are synthesized in vivo is about 1:1:20, which is the same as in the mature virus particle (Rabinowitz J E, Samulski R J (2000) Virology 278: 301-8).

The unique fragment at the N terminus of VP1 protein possesses phospholipase A2 (PLA2) activity, likely required for releasing the AAV particles from late endosomes. VP2 and VP3 are crucial for correct virion assembly (Muralidhar S, Becerra S P, Rose J A (1994), Journal of Virology 68: 170-6). More recently, however, Warrington et al. have shown VP2 to be not only unnecessary for the complete virus particle formation and an efficient infectivity, but that VP2 can tolerate large insertions in its N terminus (Warrington K H, et al. (2004), Journal of Virology 78: 6595-609). In contrast. VP1 shows no such tolerance, probably because of the presence of the PLA2 domain (Id.). The AAV capsid is composed of 60 capsid protein subunits, VP1, VP2, and VP3, that are arranged in an icosahedral symmetry in a ratio of 1:1:10, with an estimated size of 3.9 MegaDaltons. The crystal structure of the VP3 protein was determined by Xie, Bue, et al. (Xie Q, Bu W, Bhatia S, et al. (2002) Proceedings of the National Academy of Sciences of the United States of America 99: 10405-10).

Currently, 12 AAV serotypes and nearly 100 variants have been identified in human and nonhuman primate populations. (Gao G, Zhong L, Danos O (2011) Methods Mol. Biol. 807:93-118). Serotypes can infect cells from multiple diverse tissue types. Tissue specificity, as determined by the capsid serotype and pseudotyping of AAV vectors to alter their tropism range, will likely impact to their efficacy and use in therapy.

Serotype 2 (AAV2) has been the most extensively examined to date. AAV2 presents a natural tropism towards skeletal muscles, neurons, vascular smooth muscle cells, and hepatocytes. Three cell receptors have been described for AAV2: heparan sulfate proteoglycan (HSPG), aVβ5 integrin, and fibroblast growth factor receptor 1 (FGFR-1). The first functions as a primary receptor, while the latter two have a co-receptor activity and enable AAV to enter the cell by receptor-mediated endocytosis.

Although AAV2 is the most popular serotype in various AAV-based research, it has been shown that other serotypes can be more effective as gene delivery vectors. For instance, AAV6 appears much better in infecting airway epithelial cells, AAV7 presents very high transduction rate of murine skeletal muscle cells (similarly to AAV1 and AAV5), AAV8 is superb in transducing hepatocytes, and AAV1 and 5 were shown to be very efficient in gene delivery to vascular endothelial cells. In the brain, most AAV serotypes show neuronal tropism, while AAV5 also transduces astrocytes. AAV6, a hybrid of AAV1 and AAV2, also shows lower immunogenicity than AAV2. Serotypes can differ with the respect to the receptors they are bound to. For example, AAV4 and AAV5 transduction can be inhibited by soluble sialic acids (of different form for each of these serotypes), and AAV5 was shown to enter cells via the platelet-derived growth factor receptor. Currently, rAAV8 and rAAV9 show the most prominent features relevant to therapeutic use relative to all other serotypes and under undisturbed physiological conditions. (Gao, G, Zhong L, and Danos O (2011) Methods Mol. Biol. 807:93-118).

There are several steps in the AAV infection cycle, from infecting a cell to producing new infectious particles. These are: 1. attachment to the cell membrane; 2. receptor-mediated endocytosis; 3. endosomal trafficking; 4. escape from the late endosome or lysosome; 5. translocation to the nucleus; 6. uncoating; 7. formation of double-stranded DNA replicative form of the AAV genome; 8. expression of rep genes; 9. genome replication; 10. expression of cap genes, synthesis of progeny ssDNA particles; 11. assembly of complete virions, and; 12. release from the infected cell. These steps may differ depending on the host cell type, which, in part, contributes to the defined and quite limited native tropism of AAV. Replication of the virus can also, even in regards to the same cell type, be dependent on the cell's cycle phase at the time of infection.

The characteristic feature of the adeno-associated virus is a deficiency in replication and thus, its inability to multiply in unaffected cells. The first factor described as providing successful generation of new AAV particles was the adenovirus, from which the AAV name originated. It was then shown that AAV replication is facilitated by selected proteins derived from the adenovirus genome, by other viruses such as HSV, or by genotoxic agents, such as UV irradiation or hydroxyurea. The minimal set of the adenoviral genes required for efficient generation of progeny AAV particles were discovered by Matsushita, Ellinger et al. (Matsushita T, Elliger S, Elliger C, et al. (1998) Gene Therapy 5: 938-45). This discovery paved the way for new production methods of recombinant AAV, which do not require adenoviral co-infection of the AAV-producing cells. In the absence of helper virus or genotoxic factors, AAV DNA can either integrate into the host genome, or persist in episomal form. In the former case integration is mediated by Rep78 and Rep68 proteins and requires the presence of ITRs flanking the region being integrated. In mice, the AAV genome has been observed persisting for long periods in quiescent tissues, such as skeletal muscles, in episomal form (a circular head-to-tail conformation).

The present disclosure relates to the use of recombinant DNA constructs (e.g., plasmids) and more specifically to rAAV vectors to deliver engineered, site-specific nucleases.

In a particular embodiment of the invention, the DNA break-inducing agent is an engineered homing endonuclease (also called a “meganuclease”). Homing endonucleases are a group of naturally-occurring nucleases, which recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006) Q. Rev. Biophys. 38: 49-95).

Homing endonucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001) Nucleic Acids Res. 29(18): 3757-3774). The LAGLIDADG homing endonucleases with a single copy of the LAGLIDADG motif form homodimers, whereas members with two copies of the LAGLIDADG motif are found as monomers.

I-CreI is a member of the LAGLIDADG family of homing endonucleases, which recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004) J Mol Biol 342: 31-41; Chames et al. (2005) Nucleic Acids Res. 33: e178; Seligman et al. (2002) Nucleic Acids Res. 30: 3870-9; Arnould et al. (2006) J Mol. Biol. 355: 443-58). More recently, a method of rationally-designing mono-LAGLIDADG homing endonucleases capable of comprehensively redesigning I-CreI and other homing endonucleases to target widely-divergent DNA sites, including sites in mammalian, yeast, plant, bacterial, and viral genomes has been described (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li, et al. (2009) Nucleic Acids Res. 37:1650-62; Grizot, et al. (2009) Nucleic Acids Res. 37:5405-19.). Thus, a functional “single-chain” meganuclease can be expressed from a single transcript. By delivering genes encoding two different single-chain meganucleases to the same cell, it is possible to simultaneously cut two different sites. This, coupled with the extremely low frequency of off-target cutting observed with engineered meganucleases, makes them the preferred endonuclease for the present invention.

For many applications, it is necessary to deliver (a) gene(s) encoding engineered endonuclease(s) to the target cell or organism. For in vivo applications, rAAV is a preferred delivery vector. However, rAAV vectors have long persistence times in many cell types, particularly non-dividing cells. Such persistence can activate immune response within the cell and cause disruption. Genome editing using engineered endonucleases requires only a short burst of endonuclease expression such that the endonuclease protein accumulates to a sufficient intracellular concentration to cut its recognition sequence in the genome. Long-term expression of an endonuclease can result in unintended off-target DNA cutting or in an immune response directed toward cells expressing the foreign nuclease protein. Thus, there is a need for rAAV vectors encoding site-specific gene editing endonucleases in which the persistence time of the vector is limited, off target cutting is reduced, whilst on target cutting is maintained.

### SUMMARY OF THE INVENTION

Disclosed herein is a self-limiting recombinant virus having limited persistence time in a cell or organism due to the presence of two or more recognition sequences for an engineered nuclease within the virus. Also disclosed herein are methods for using the self-limiting recombinant virus for genome editing applications.

It is understood that any of the embodiments described below can be combined in any desired way, and any embodiment or combination of embodiments can be applied to each of the aspects described below, unless the context indicates otherwise.

In one aspect, the present disclosure provides a recombinant DNA construct containing a polynucleotide, wherein the polynucleotide contains: (a) a first nucleic acid sequence encoding a first engineered nuclease; (b) a first promoter operably linked to the first nucleic acid sequence encoding the first engineered nuclease, wherein the promoter is positioned 5′ upstream of the first nucleic acid sequence and drives expression of the first engineered nuclease in a target cell; and (c) two or more engineered nuclease construct recognition sequences.

In some embodiments of the recombinant DNA construct, the polynucleotide contains a nuclear localization signal that is positioned 5′ upstream of the first nucleic acid sequence encoding the first engineered nuclease. In alternative embodiments of the recombinant DNA construct, the polynucleotide contains a nuclear localization signal that is positioned 3′ downstream of the first nucleic acid sequence encoding the first engineered nuclease.

In some embodiments of the recombinant DNA construct, the polynucleotide contains an intron that is positioned within the first nucleic acid sequence encoding the first engineered nuclease. In some such embodiments of the recombinant DNA construct, the intron is positioned 3′ downstream of the nuclear localization signal and 5′ upstream of the first nucleic acid sequence encoding the first engineered nuclease.

In some embodiments of the recombinant DNA construct, at least one of the two or more engineered nuclease construct recognition sequences is positioned 3′ downstream of the intron. In some embodiments of the recombinant DNA construct, at least one of the two or more engineered nuclease construct recognition sequences is positioned 5′ upstream of the intron. In some embodiments of the recombinant DNA construct, at least one of the two or more engineered nuclease construct recognition sequences is positioned within the intron.

In some embodiments of the recombinant DNA construct, the first promoter is a tissue-specific promoter, a species-specific promoter, a constitutive promoter or an inducible promoter.

In some such embodiments of the recombinant DNA construct, the tissue-specific promoter comprises a liver-specific promoter, an ocular-specific promoter, a central nervous system (CNS)-specific promoter, a lung specific promoter, a skeletal muscle-specific promoter, a heart-specific promoter, or a kidney-specific promoter. In certain embodiments of the recombinant DNA construct, the tissue-specific promoter is a liver-specific promoter. In particular embodiments of the recombinant DNA construct, the liver-specific promoter comprises a human thyroxine binding globulin (TBG) promoter, a human alpha-1 antitrypsin promoter, a hybrid liver specific promoter, or an apolipoprotein A-II promoter. In certain embodiments of the recombinant DNA construct, the tissue-specific promoter is an ocular-specific promoter. In particular embodiments of the recombinant DNA construct, the ocular-specific promoter comprises human G-protein-coupled receptor protein kinase 1 (GRK1) promoter.

In some embodiments of the recombinant DNA construct, the constitutive promoter is a native promoter. In alternative embodiments of the recombinant DNA construct, the constitutive promoter is a composite promoter.

In some embodiments of the recombinant DNA construct, the first promoter is an inducible promoter, and in such embodiments, the polynucleotide further comprises a nucleic acid sequence encoding a ligand-inducible transcription factor, wherein the ligand-inducible transcription factor regulates activation of the first promoter.

In some embodiments of the recombinant DNA construct, the polynucleotide further comprises a second nucleic acid sequence encoding a second engineered nuclease. In some such embodiments of the recombinant DNA construct, the first and the second engineered nucleases are different types of nucleases. In further embodiments of the recombinant DNA construct, the polynucleotide further comprises a second promoter operably linked to the second nucleic acid sequence encoding the second engineered nuclease.

In some embodiments of the recombinant DNA construct, the two or more engineered nuclease construct recognition sequences are non-identical. In alternative embodiments of the recombinant DNA construct, the two or more engineered nuclease construct recognition sequences are identical.

In some embodiments of the recombinant DNA construct, the first engineered nuclease binds and cleaves a genomic recognition sequence in a target cell and at least one of the two or more engineered nuclease construct recognition sequences, wherein the genomic recognition sequence is identical to at least one of the two or more engineered nuclease construct recognition sequences. In some such embodiments of the recombinant DNA construct, the first engineered nuclease binds and cleaves a genomic recognition sequence in a target cell and all of the two or more engineered nuclease construct recognition sequences, wherein the genomic recognition sequence is identical to the two or more engineered nuclease construct recognition sequences.

In some embodiments of the recombinant DNA construct, the first engineered nuclease binds and cleaves a genomic recognition sequence in a target cell, wherein the first engineered nuclease binds and cleaves at least one of the two or more engineered nuclease construct recognition sequences, wherein the genomic recognition sequence is identical to at least one of the two or more engineered nuclease construct recognition sequences, and wherein one or more second engineered nucleases binds and cleaves at least one of the two or more engineered nuclease construct recognition sequences.

In some embodiments of the recombinant DNA construct, the first engineered nuclease binds and cleaves a genomic recognition sequence in a target cell, wherein the genomic recognition sequence is not identical to the two or more engineered nuclease construct recognition sequences. In some such embodiments of the recombinant DNA construct, the first engineered nuclease cleaves at least one of the two or more engineered nuclease construct recognition sequences at about a 50% to about a 90% cleavage rate compared to a cleavage rate of the first engineered nuclease for the genomic recognition sequence. In certain embodiments of the recombinant DNA construct, the first engineered nuclease does not substantially cleave the two or more engineered nuclease construct recognition sequences.

In some embodiments of the recombinant DNA construct, a second engineered nuclease binds and cleaves at least one of the two or more engineered nuclease construct recognition sequences. In some such embodiments of the recombinant DNA construct, a second engineered nuclease binds and cleaves all of the engineered nuclease construct recognition sequences. In certain embodiments of the recombinant DNA construct, the second engineered nuclease cleaves the genomic recognition sequence at about a 50% to about a 90% cleavage rate compared to a cleavage rate of the second engineered nuclease for at least one of the two or more engineered nuclease construct recognition sequences. In certain embodiments of the recombinant DNA construct, the second engineered nuclease does not substantially cleave the genomic recognition sequence.

In some embodiments of the recombinant DNA construct, the genomic recognition sequence and at least one of the two or more engineered nuclease construct recognition sequences comprise different center sequences but identical recognition half-site sequences.

In some embodiments, the recombinant DNA construct further comprises a polyA sequence positioned 3′ downstream of the first nucleic acid sequence encoding the first engineered nuclease.

In some embodiments, the recombinant DNA construct further comprises a protein degradation peptide encoding sequence positioned 3′ downstream of the first nucleic acid sequence encoding the first engineered nuclease. In some such embodiments of the recombinant DNA construct, the protein degradation peptide comprises a PEST, an intracellular protein degradation signal sequence, a degron sequence, or an ubiquitin sequence.

In some embodiments of the recombinant DNA construct, the protein degradation peptide encoding sequence is positioned 5′ upstream of at least one of the two or more engineered nuclease construct recognition sequences. In certain embodiments of the recombinant DNA construct, the protein degradation peptide encoding sequence is positioned 3′ downstream of at least one of the two or more engineered nuclease construct recognition sequences.

In some embodiments, the recombinant DNA construct comprises a first engineered nuclease construct recognition sequence and a second engineered nuclease construct recognition sequence. In some such embodiments of the recombinant DNA construct, distance between the first and the second engineered nuclease construct recognition sequences is at least 1000 nucleotides (e.g., at least 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides. In other embodiments of the recombinant DNA construct, distance between the first and the second engineered nuclease construct recognition sequences is about 1000-2500 (e.g., about 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides.

In certain embodiments, the recombinant DNA construct comprises a polynucleotide, wherein the polynucleotide comprises from 5′ to 3′: (i) a first promoter sequence, wherein the first promoter sequence is operably linked to a first nucleic acid sequence encoding a first engineered nuclease and drives expression of the first engineered nuclease in a target cell; (ii) a first engineered nuclease construct recognition sequence positioned 3′ downstream of the first promoter; (iii) a nuclear localization signal positioned 3′ downstream of the first engineered nuclease construct recognition sequence; (iv) an intron positioned 3′ downstream of the nuclear localization signal and 5′ upstream of the first nucleic acid sequence encoding the first engineered nuclease; (v) a second engineered nuclease construct recognition sequence positioned 3′ downstream of the first nucleic acid sequence encoding the first engineered nuclease; and (vi) a polyA sequence positioned 3′ downstream of the second engineered nuclease construct recognition sequence.

In other embodiments, the recombinant DNA construct comprises a polynucleotide, wherein the polynucleotide comprises from 5′ to 3′: (i) a first promoter sequence, wherein the first promoter sequence is operably linked to a first nucleic acid sequence encoding a first engineered nuclease and drives expression of the first engineered nuclease in a target cell; (ii) a first engineered nuclease construct recognition sequence positioned 3′ downstream of the first promoter; (iii) a nuclear localization signal positioned 3′ downstream of the first engineered nuclease construct recognition sequence; (iv) an intron positioned 3′ downstream of the nuclear localization signal and 5′ upstream of the first nucleic acid sequence encoding the first engineered nuclease; (v) a protein degradation peptide encoding sequence positioned 3′ downstream of the first nucleic acid sequence encoding the first engineered nuclease; (vi) a second engineered nuclease construct recognition sequence positioned 3′ downstream of the protein degradation peptide encoding sequence; and (vii) a polyA sequence positioned 3′ downstream of the second engineered nuclease construct recognition sequence.

In other embodiments, the recombinant DNA construct comprises a polynucleotide, wherein the polynucleotide comprises from 5′ to 3′: (i) a first promoter sequence, wherein the first promoter sequence is operably linked to a first nucleic acid sequence encoding a first engineered nuclease and drives expression of the first engineered nuclease in a target cell; (ii) a nuclear localization signal positioned 3′ downstream of the first promoter; (iii) an intron positioned 3′ downstream of the nuclear localization signal and 5′ upstream of the first nucleic acid sequence encoding the first engineered nuclease; (iv) a first engineered nuclease construct recognition sequence positioned within the intron; (v) a protein degradation peptide encoding sequence positioned 3′ downstream of the first nucleic acid sequence encoding the first engineered nuclease; (vi) a second engineered nuclease construct recognition sequence positioned 3′ downstream of the protein degradation peptide encoding sequence; and (vii) a polyA sequence positioned 3′ downstream of the second engineered nuclease construct recognition sequence.

In some embodiments, the recombinant DNA construct comprises a first engineered nuclease construct recognition sequence, a second engineered nuclease construct recognition sequence, and a third engineered nuclease construct recognition sequence. In some such embodiments, distance between the first and the second engineered nuclease construct recognition sequences is at least 50 (e.g., at least 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides and distance between the second and the third engineered nuclease construct recognition sequences is at least 1000 (e.g., at least 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides. In other embodiments of the recombinant DNA construct, the distance between the first and the second nuclease recognition sequences is about 50-2500 (e.g., about 50-75, 75-100, 100-150, 150-200, 200-250, 250-300, 300-350, 350-400, 400-450, 450-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides, and distance between the second and the third engineered nuclease construct recognition sequences is about 1000-2500 (e.g., about 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides.

In certain embodiments, the recombinant DNA construct comprises a polynucleotide, wherein the polynucleotide comprises from 5′ to 3′: (i) a first promoter sequence, wherein the first promoter sequence is operably linked to a first nucleic acid sequence encoding a first engineered nuclease and drives expression of the first engineered nuclease in a target cell; (ii) a first engineered nuclease construct recognition sequence positioned 3′ downstream of the first promoter; (iii) a nuclear localization signal positioned 3′ downstream of the first engineered nuclease construct recognition sequence; (iv) an intron positioned 3′ downstream of the nuclear localization signal and 5′ upstream of the first nucleic acid sequence encoding the first engineered nuclease; (v) a second engineered nuclease construct recognition sequence positioned within the intron; (vi) a third engineered nuclease construct recognition sequence positioned 3′ downstream of the first nucleic acid sequence encoding the first engineered nuclease; and (vii) a polyA sequence positioned 3′ downstream of the third engineered nuclease construct recognition sequence.

In other embodiments, the recombinant DNA construct comprises a polynucleotide, wherein the polynucleotide comprises from 5′ to 3′: (i) a first promoter sequence, wherein the first promoter sequence is operably linked to a first nucleic acid sequence encoding a first engineered nuclease and drives expression of the first engineered nuclease in a target cell; (ii) a first engineered nuclease construct recognition sequence positioned 3′ downstream of the first promoter; (iii) a nuclear localization signal positioned 3′ downstream of the first engineered nuclease construct recognition sequence; (iv) an intron positioned 3′ downstream of the nuclear localization signal and 5′ upstream of the first nucleic acid sequence encoding the first engineered nuclease; (v) a second engineered nuclease construct recognition sequence positioned within the intron; (vi) a protein degradation peptide encoding sequence positioned 3′ downstream of the first nucleic acid sequence encoding the first engineered nuclease; (vii) a third engineered nuclease construct recognition sequence positioned 3′ downstream of the protein degradation peptide encoding sequence; and (viii) a polyA sequence positioned 3′ downstream of the third engineered nuclease construct recognition sequence.

In some embodiments of the recombinant DNA construct, the engineered nuclease comprises one or more of an engineered meganuclease, a TALEN, a compact TALEN, a zinc finger nuclease, a CRISPR/Cas9 nuclease, or a megaTAL. In particular embodiments of the recombinant DNA construct, the engineered nuclease comprises an engineered meganuclease.

In another aspect, the present disclosure provides a plasmid comprising the recombinant DNA construct described hereinabove.

In another aspect, the present disclosure provides a recombinant virus comprising the recombinant DNA construct described hereinabove.

In some embodiments, the recombinant virus is a recombinant adenovirus, a recombinant lentivirus, a recombinant retrovirus, or a recombinant adeno-associated virus (AAV). In certain embodiments, the recombinant virus is a recombinant AAV. In some such embodiments, the recombinant AAV has an AAV8 serotype. In other embodiments, the recombinant AAV has an AAV5 serotype. In alternative embodiments, the recombinant AAV has an AAV2 serotype.

In another aspect, the present disclosure provides a pharmaceutical composition comprising a pharmaceutically acceptable carrier and a plasmid described herein.

In another aspect, the present disclosure provides a pharmaceutical composition comprising a pharmaceutically acceptable carrier and a recombinant DNA construct described herein.

In another aspect, the present disclosure provides a pharmaceutical composition comprising a pharmaceutically acceptable carrier and a recombinant virus described herein.

In another aspect, the present disclosure provides a method of cleaving a target site in genome of a target cell, by introducing a plasmid or a recombinant virus described hereinabove.

In some embodiments of the method, cleavage of the two or more engineered nuclease construct recognition sequences by the engineered nuclease in the target cell increases on-target cleavage of the genome of the target cell by at least 10% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the engineered nuclease. In other embodiments of the method, cleavage of the two or more engineered nuclease construct recognition sequences by the engineered nuclease in the target cell increases on-target cleavage of the genome of the target cell by about 10-90% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the engineered nuclease.

In some embodiments of the method, cleavage of the two or more engineered nuclease construct recognition sequences by the engineered nuclease in the target cell decreases off-target cleavage of the genome of the target cell by at least 10% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the engineered nuclease. In other embodiments of the method, cleavage of the two or more engineered nuclease construct recognition sequences by the engineered nuclease in the target cell decreases off-target cleavage of the genome of the target cell by about 10-90% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the engineered nuclease.

In some embodiments of the method, cleavage of the two or more engineered nuclease construct recognition sequences by the engineered nuclease in the target cell reduces the persistence time of the plasmid or the recombinant virus in the target cell when compared to a control plasmid or control recombinant virus that does not comprise two or more engineered nuclease construct recognition sequences cleaved by the engineered nuclease. In some such embodiments of the method, the persistence time in the target cell is less than 10 weeks. In certain embodiments of the method, the persistence time in the target cell is less than 6 weeks. In particular embodiments of the method, the persistence time in the target cell is about 2 weeks.

In some embodiments of the method, the engineered nuclease binds and cleaves a genomic recognition sequence in the target cell, and wherein following cleavage of the two or more engineered nuclease construct recognition sequences, integration of the plasmid or the recombinant virus into the genome of the target cell is reduced by at least 10% following at least 2 weeks, at least 6 weeks, or at least 10 weeks after introduction of the plasmid or the recombinant virus into the target cell, when compared to introducing a control plasmid or control recombinant virus that does not comprise two or more engineered nuclease construct recognition sequences cleaved by the engineered nuclease. In other embodiments of the method, the engineered nuclease binds and cleaves a genomic recognition sequence in the target cell, and wherein following cleavage of the two or more engineered nuclease construct recognition sequences, integration of the plasmid or the recombinant virus into the genome of the target cell is reduced by about 10-90% following at least 2 weeks, at least 6 weeks, or at least 10 weeks after introduction of the plasmid or the recombinant virus into the target cell, when compared to introducing a control plasmid or control recombinant virus that does not comprise two or more engineered nuclease construct recognition sequences cleaved by the engineered nuclease.

In some embodiments of the method, cleavage of the two or more engineered nuclease construct recognition sequences by the engineered nuclease in the target cell reduces mRNA and/or protein expression of the engineered nuclease in the target cell by at least 10% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the first engineered nuclease. In other embodiments of the method, cleavage of the two or more engineered nuclease construct recognition sequences by the engineered nuclease in the target cell reduces mRNA and/or protein expression of the engineered nuclease in the target cell by about 10-90% following at least 2 weeks, at least 6 weeks, or at least 10 weeks after introduction of the plasmid or the recombinant virus into the target cell, when compared to introduction of a control plasmid or control recombinant virus that does not comprise two or more engineered nuclease construct recognition sequences cleaved by the first engineered nuclease.

In some embodiments of the method, cleavage of the two or more engineered nuclease construct recognition sequences by the engineered nuclease in the target cell reduces copy number of the plasmid or the recombinant virus in the target cell following at least 2 weeks, at least 6 weeks, or at least 10 weeks after introduction of the plasmid or the recombinant virus into the target cell by at least 10%, when compared to a control plasmid or control recombinant virus that does not comprise two or more engineered nuclease construct recognition sequences cleaved by the engineered nuclease. In other embodiments of the method, cleavage of the two or more engineered nuclease construct recognition sequences by the engineered nuclease in the target cell reduces copy number of the plasmid or the recombinant virus in the target cell following at least 2 weeks, at least 6 weeks, or at least 10 weeks after introduction of the plasmid or the recombinant virus into the target cell by about 10-90%, when compared to a control plasmid or control recombinant virus that does not comprise two or more engineered nuclease construct recognition sequences cleaved by the engineered nuclease.

In some embodiments of the method, cleavage of the two or more engineered nuclease construct recognition sequences by the engineered nuclease in the target cell reduces immunogenic and genotoxic effect of the plasmid or said recombinant virus in the target cell by at least 10% following at least 2 weeks, at least 6 weeks, or at least 10 weeks after introduction of the plasmid or the recombinant virus into the target cell, when compared to a control plasmid or control recombinant virus that does not comprise two or more engineered nuclease construct recognition sequences cleaved by the engineered nuclease. In other embodiments of the method, cleavage of the two or more engineered nuclease construct recognition sequences by the engineered nuclease in the target cell reduces immunogenic and genotoxic effect of the plasmid or the recombinant virus in the target cell by about 10-90% following at least 2 weeks, at least 6 weeks, or at least 10 weeks after introduction of the plasmid or the recombinant virus into the target cell, when compared to a control plasmid or control recombinant virus that does not comprise two or more engineered nuclease construct recognition sequences cleaved by the engineered nuclease. In certain embodiments of the method, the genotoxic effect comprises translocations, inversions, and/or indels.

In some embodiments of the method, the target cell is a eukaryotic cell. In particular embodiments of the method, the eukaryotic cell is a mammalian cell. In specific embodiments of the method, the eukaryotic cell is a human cell. In alternative embodiments of the method, the eukaryotic cell is a plant cell.

In another aspect, the present disclosure provides a method for producing a genetically-modified eukaryotic cell having a disrupted target sequence in a genome of the genetically modified eukaryotic cell, by: introducing into the eukaryotic cell the recombinant DNA construct described hereinabove, wherein the engineered nuclease is expressed in the eukaryotic cell; wherein the engineered nuclease produces a cleavage site in the genome at a genomic recognition sequence, and wherein the target sequence is disrupted by non-homologous end-joining at the cleavage site. In some embodiments of the method the first engineered nuclease binds encoded by the recombinant DNA construct and cleaves at least one of said two or more engineered nuclease construct recognition sequences. In some embodiments of the method, the first engineered nuclease encoded by the recombinant DNA construct binds and cleaves all of the two or more engineered nuclease construct recognition sequences.

In some embodiments of the method, the recombinant DNA construct is introduced into the eukaryotic cell by a plasmid or a recombinant virus described hereinabove.

In some embodiments of the method, the eukaryotic cell is a mammalian cell. In particular embodiments of the method, the eukaryotic cell is a human cell. In alternative embodiments of the method, the eukaryotic cell is a plant cell.

In another aspect, the present disclosure provides a method for producing a genetically-modified eukaryotic cell comprising an exogenous sequence of interest inserted into a genome of the eukaryotic cell, by introducing into the eukaryotic cell one or more recombinant DNA constructs, including: (a) a recombinant DNA construct described hereinabove, wherein the engineered nuclease is expressed in the eukaryotic cell; and (b) a second recombinant DNA construct encoding the sequence of interest. In such embodiments, the engineered nuclease produces a cleavage site in the genome at a genomic recognition sequence; and the sequence of interest is inserted into the genome at the cleavage site. In some embodiments of the method the first engineered nuclease binds encoded by the recombinant DNA construct and cleaves at least one of said two or more engineered nuclease construct recognition sequences. In some embodiments of the method, the first engineered nuclease encoded by the recombinant DNA construct binds and cleaves all of the two or more engineered nuclease construct recognition sequences.

In some embodiments of the method, the second recombinant DNA construct further comprises sequences homologous to sequences flanking the cleavage site and the sequence of interest is inserted at the cleavage site by homologous recombination.

In some embodiments of the method, the recombinant DNA construct is introduced into the eukaryotic cell by a recombinant virus. In some embodiments of the method, the second recombinant DNA construct is introduced into the eukaryotic cell by a recombinant virus.

In some embodiments of the method, the eukaryotic cell is a mammalian cell. In particular embodiments of the method, the eukaryotic cell is a human cell. In alternative embodiments of the method, the eukaryotic cell is a plant cell.

In another aspect, the present disclosure provides a genetically-modified eukaryotic cell that is prepared by the method described hereinabove.

### BRIEF DESCRIPTION OF THE FIGURES

FIG. 1 provides schematic diagrams of six self-limiting AAV (AAV) vectors (NoTS-PEST, 2TS1, 2TS1-PEST, 2TS2-PEST, 3TS, and 3TS-PEST) with 2-3 copies of nuclease recognition sequence, which is also referred to herein as a nuclease target site (TS) or alternatively as an engineered nuclease construct recognition sequence, and/or PEST tag inserted and one additional construct (NoTS) without any insertion. The vector genome is shown with inverted terminal repeats (ITRs) at the ends. Each vector encodes a site-specific nuclease, and in all constructs, the nuclease expression is under control of a TBG promoter.

FIGS. 2A-2B provide results from experiments directed at determining protein expression of nuclease in liver tissues of mice that were injected with AAV vectors (NoTS, NoTS-PEST, 2TS1, 2TS1-PEST, 2TS2-PEST, 3TS, or 3TS-PEST), as determined by western blot analysis at 2, 6, or 10 weeks following the AAV injection. FIG. 2A provides images from the western blot analysis. FIG. 2B is a graph showing quantification of nuclease expression levels by measuring intensity of the bands from the western blot analysis.

FIGS. 3A-3B provide results from experiments directed at determining mRNA expression of nuclease in liver tissues of mice that were injected with AAV vectors (NoTS, NoTS-PEST, 2TS1, 2TS1-PEST, 2TS2-PEST, 3TS, or 3TS-PEST), as determined by qRT-PCR analysis at 2, 6, or 10 weeks following the AAV injection. FIG. 3A is a graph showing mRNA level of nuclease relative to GAPDH. FIG. 3B is a graph showing mRNA level of nuclease encoded by different AAV vectors normalized to the mRNA level of nuclease encoded by NoTS.

FIGS. 4A-4C provide results from experiments directed at determining absolute AAV copy numbers per diploid genome in liver tissues of mice that were injected with AAV vectors (NoTS, NoTS-PEST, 2TS1, 2TS1-PEST, 2TS2-PEST, 3TS, or 3TS-PEST), as determined by qPCR analysis using 3 primer pairs (SV40, TBG and TS1) at 2, 6, or 10 weeks following the AAV injection. FIG. 4A is a graph showing absolute AAV copy numbers per diploid genome, as determined using the SV40 primers. FIG. 4B is a graph showing absolute AAV copy numbers per diploid genome, as determined using the TBG primers. FIG. 4C is a graph showing absolute AAV copy numbers per diploid genome, as determined using the SV40, TBG, and TS1 primers.

FIGS. 5A-5C provide results from experiments directed at determining AAV copy number normalized to copy number of NoTS in liver tissues of mice that were injected with AAV vectors (NoTS, NoTS-PEST, 2TS1, 2TS1-PEST, 2TS2-PEST, 3TS, or 3TS-PEST), wherein the AAV copy number is determined by qPCR analysis using 3 primer pairs (SV40, TBG, and TS1) at 2, 6, or 10 weeks following the AAV injection. FIG. 5A is a graph showing AAV copy number relative to copy number of NoTS, as determined using the SV40 primers. FIG. 5B is a graph showing AAV copy number relative to copy number of NoTS, as determined using the TBG primers. FIG. 5C is a graph showing AAV copy number relative to copy number of NoTS, as determined using the and TS1 primers.

FIGS. 6A-6B provide results from experiments directed at determining insertion and deletion (indel) rates as a measure of nuclease activity in gDNA extracted from liver tissues of mice that were injected with AAV vectors (NoTS, NoTS-PEST, 2TS1, 2TS1-PEST, 2TS2-PEST, 3TS, or 3TS-PEST), wherein the indel rate is determined by digital droplet PCR (ddPCR), at 2, 6, or 10 weeks following the AAV injection. FIG. 6A is a graph depicting absolute indel rates as a measure of nuclease activity of the AAV vectors. FIG. 6B is a graph depicting nuclease activity of the AAV vectors relative to nuclease activity of NoTS.

FIGS. 7A-7B provide results from experiments directed at determining indel rates as a measure of nuclease activity in gDNA extracted from liver tissues of mice that were injected with AAV vectors (NoTS, NoTS-PEST, 2TS1, 2TS1-PEST, 2TS2-PEST, 3TS, or 3TS-PEST), wherein the indel rate is determined by on-target amplicon sequencing (amplicon-seq), at 2, 6, or 10 weeks following the AAV injection. FIG. 7A is a graph depicting indel rates as a measure of on-target nuclease activity of the AAV vectors. FIG. 7B is a graph showing correlation between indel rates results determined by ddPCR and indel rate results determined by amplicon-seq.

FIGS. 8A-8B provide results from experiments directed at correlating genome indel rates and AAV indel rates. FIG. 8A is a graph depicting indel rates in AAV genome. FIG. 8B is a graph showing correlation between AAV indel rates (as described in FIG. 8A) and genome indel rates or on-target indel rates (as described in FIG. 7A).

FIG. 9 depicts three amplicons containing nuclease off-target sites (off-target 1, 2, and 4) in mouse genome that were PCR amplified with mouse gDNA as template and visualized on 1% agarose TAE gel.

FIGS. 10A-10C provide the results from experiments directed at determining nuclease activity of AAV vectors NoTS, NoTS-PEST, 2TS1, 2TS1-PEST, 2TS2-PEST, 3TS, or 3TS-PEST at three identified off-target sites 1 (FIG. 10A), 2 (FIG. 10B), and 4 (FIG. 10C) in gDNA extracted from liver tissues of mice that were injected with the AAV vectors, wherein the nuclease activity determined at 2, 6, or 10 weeks following the AAV injection.

FIGS. 11A-11D provide results from experiments directed at determining relative nuclease activity at same nuclease protein levels for AAV vectors NoTS, 2TS1, or 3TS, on on-target site and off-target sites 1, 2, and 4 in gDNA extracted from liver tissues of mice that were injected with the AAV vectors, wherein the nuclease activity determined at 2, 6, or 10 weeks following the AAV injection is normalized against the nuclease protein levels determined by western blot analysis. FIG. 11A is a graph depicting relative nuclease activity of the AAV vectors on on-target site at the same nuclease protein level of NoTS group. FIG. 11B is a graph depicting relative nuclease activity of the AAV vectors on off-target site 1 at the same nuclease protein level of NoTS group. FIG. 11C is a graph depicting relative nuclease activity of the AAV vectors on off-target site 2 at the same nuclease protein level of NoTS group. FIG. 11D is a graph depicting relative nuclease activity of the AAV vectors on off-target site 4 at the same nuclease protein level of NoTS group.

FIGS. 12A-12D provide results from experiments directed at determining relative nuclease activity at same nuclease mRNA levels for AAV vectors NoTS, NoTS-PEST, 2TS1, 2TS1-PEST, 2TS2-PEST, 3TS, or 3TS-PEST, on on-target site and off-target sites 1, 2, and 4 in gDNA extracted from liver tissues of mice that were injected with the AAV vectors, wherein the nuclease activity determined at 2, 6, or 10 weeks following the AAV injection is normalized against the nuclease mRNA levels determined by qRT-PCR analysis. FIG. 12A is a graph depicting relative nuclease activity of the AAV vectors on on-target site at the same nuclease mRNA level of NoTS group. FIG. 12B is a graph depicting relative nuclease activity of the AAV vectors on off-target site 1 at the same nuclease mRNA level of NoTS group. FIG. 12C is a graph depicting relative nuclease activity of the AAV vectors on off-target site 2 at the same nuclease mRNA level of NoTS group. FIG. 12D is a graph depicting relative nuclease activity of the AAV vectors on off-target site 4 at the same nuclease mRNA level of NoTS group.

### BRIEF DESCRIPTION OF THE SEQUENCES

SEQ ID NO: 1 sets forth the nucleic acid sequence of the forward primer used to determine viral titers by qPCR.

SEQ ID NO: 2 sets forth the nucleic acid sequence of the reverse primer used to determine viral titers by qPCR.

SEQ ID NO: 3 sets forth the nucleic acid sequence of the forward primer used to target nuclease open reading frame (ORF).

SEQ ID NO: 4 sets forth the nucleic acid sequence of the reverse primer used to target nuclease open reading frame (ORF).

SEQ ID NO: 5 sets forth the nucleic acid sequence of the forward primer used to target mouse GAPDH gene.

SEQ ID NO: 6 sets forth the nucleic acid sequence of the reverse primer used to target mouse GAPDH gene.

SEQ ID NO: 7 sets forth the nucleic acid sequence of the forward primer of the SV40 primer pair.

SEQ ID NO: 8 sets forth the nucleic acid sequence of the reverse primer of the SV40 primer pair.

SEQ ID NO: 9 sets forth the nucleic acid sequence of the forward primer of the TBG primer pair.

SEQ ID NO: 10 sets forth the nucleic acid sequence of the reverse primer of the TBG primer pair.

SEQ ID NO: 11 sets forth the nucleic acid sequence of the forward primer of the TS1 primer pair.

SEQ ID NO: 12 sets forth the nucleic acid sequence of the reverse primer of the TS1 primer pair.

SEQ ID NO: 13 sets forth the nucleic acid sequence of the target forward primer (28-HAO21-22 F2) for indel analysis by ddPCR.

SEQ ID NO: 14 sets forth the nucleic acid sequence of the target reverse primer (27-HAO21-22 R2) for indel analysis by ddPCR.

SEQ ID NO: 15 sets forth the nucleic acid sequence of the target probe (42 HAO1 2 BHQ 1) for indel analysis by ddPCR.

SEQ ID NO: 16 sets forth the nucleic acid sequence of the reference probe (44 12REf PROBE1) for indel analysis by ddPCR.

SEQ ID NO: 17 sets forth the nucleic acid sequence of the gene specific primer (forward; 28 F2) for amplicon-seq.

SEQ ID NO: 18 sets forth the nucleic acid sequence of the gene specific primer (reverse; 27 R2) for amplicon-seq.

SEQ ID NO: 19 sets forth the nucleic acid sequence of the forward primer (oft1f) used to target Off-target 1 amplicon.

SEQ ID NO: 20 sets forth the nucleic acid sequence of the reverse primer (oft1r) used to target Off-target 1 amplicon.

SEQ ID NO: 21 sets forth the nucleic acid sequence of the forward primer (oft2f) used to target Off-target 2 amplicon.

SEQ ID NO: 22 sets forth the nucleic acid sequence of the reverse primer (oft2r) used to target Off-target 2 amplicon.

SEQ ID NO: 23 sets forth the nucleic acid sequence of the forward primer (oft4f) used to target Off-target 4 amplicon.

SEQ ID NO: 24 sets forth the nucleic acid sequence of the reverse primer (oft4r) used to target Off-target 4 amplicon.

SEQ ID NO: 25 sets forth the nucleic acid sequence of the On-target amplicon (HAO_ON). SEQ ID NO: 26 sets forth the nucleic acid sequence of the Off-target 1 amplicon (HAO_Off1).

SEQ ID NO: 27 sets forth the nucleic acid sequence of the Off-target 2 amplicon (HAO_Off2).

SEQ ID NO: 28 sets forth the nucleic acid sequence of the Off-target 4 amplicon (HAO_Off4).

SEQ ID NO: 29 sets forth the nucleic acid sequence of the self-limiting AAV vector NoTS.

SEQ ID NO: 30 sets forth the nucleic acid sequence of the self-limiting AAV vector NoTS-PEST.

SEQ ID NO: 31 sets forth the nucleic acid sequence of the self-limiting AAV vector 2TS1.

SEQ ID NO: 32 sets forth the nucleic acid sequence of the self-limiting AAV vector 2TS1-PEST.

SEQ ID NO: 33 sets forth the nucleic acid sequence of the self-limiting AAV vector 2TS2-PEST.

SEQ ID NO: 34 sets forth the nucleic acid sequence of the self-limiting AAV vector 3TS.

SEQ ID NO: 35 sets forth the nucleic acid sequence of the self-limiting AAV vector 3TS-PEST.

SEQ ID NO: 36 sets forth the nucleic acid sequence of the human growth hormone (HGH) intron.

SEQ ID NO: 37 sets forth the nucleic acid sequence of the SV40 large T antigen intron.

SEQ ID NO: 38 sets forth the sequence of HAO1-2L30 meganuclease without a signal sequence.

### DETAILED DESCRIPTION OF THE INVENTION

### 1.1. References and Definitions

The patent and scientific literature referred to herein establishes knowledge that is available to those of skill in the art. The entire disclosures of the issued U.S. patents, pending applications, published foreign applications, and references, including GenBank database sequences, that are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

Reference will now be made in detail to particular embodiments of the self-limiting viral vector, examples of which are illustrated in the accompanying drawings.

As used herein, the term “cell” refers to a cell, whether it be part of a cell line, tissue, or organism. “Cell” may refer to cells of microbial, plant, insect, or animal (mammalian, reptilian, avian, or otherwise) type, and where necessary, is specified.

As used herein, the terms “nuclease” and “endonuclease” are used interchangeably to refer to naturally-occurring or engineered enzymes, which cleave a phosphodiester bond within a polynucleotide chain.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein. As used herein, the term “single-chain meganuclease” refers to a polypeptide comprising a pair of meganuclease subunits joined by a linker. A single-chain meganuclease has the organization: N-terminal subunit-Linker-C-terminal subunit. The two meganuclease subunits, each of which is derived from I-CreI, will generally be non-identical in amino acid sequence and will recognize non-identical DNA sequences. Thus, single-chain meganucleases typically cleave pseudo-palindromic or non-palindromic recognition sequences. A single chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “site specific endonuclease” means a meganuclease, TALEN, Compact TALEN, Zinc-Finger Nuclease, or CRISPR.

As used herein, the term “compact TALEN” refers to an endonuclease comprising a DNA-binding domain with one or more TAL domain repeats fused in any orientation to any portion of the I-TeVI homing endonuclease or any of the endonucleases listed in Table 2 in U.S. Application No. 20130117869 (which is incorporated by reference in its entirety), including but not limited to MmeI, EndA, EndI, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats. As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, 51 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zincion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, itis possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site. As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “gene” refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “endogenous” in reference to a nucleotide sequence or protein is intended to mean a sequence or protein that is naturally comprised within or expressed by a cell.

As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “expression” refers to the transcription and/or translation of a particular nucleotide sequence driven by a promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “poly(A)” is a series of adenosines attached by polyadenylation to the mRNA. In a particular embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, the term “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors, lentiviral vectors, adenoviral vectors, and adeno-associated viral vectors (AAV).

As used herein, the terms “adeno-associated viral particle” or “adeno-associated virus particle” or “AAV particle” refer to an adeno-associated capsid shell that may or may not comprise a viral genome encapsulated therein.

As used herein, the term “genetically-modified” refers to a cell or organism in which, or in an ancestor of which, a genomic DNA sequence has been deliberately modified by recombinant technology. As used herein, the term “genetically-modified” encompasses the terms “recombinant” or “transgenic.”

As used herein, with respect to a protein, the term “recombinant” means having an altered amino acid sequence as a result of the application of genetic engineering techniques to nucleic acids which encode the protein, and cells or organisms which express the protein. With respect to a nucleic acid, the term “recombinant” means having an altered nucleic acid sequence as a result of the application of genetic engineering techniques. Genetic engineering techniques include, but are not limited to: PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant. As used herein, the term “engineered” is synonymous with the term “recombinant.”

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or maybe used in conjunction with a vector.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, “off-target cleavage” refers to single stranded or double stranded cleavage of any site in the genome of a target cell that is not a recognition sequence of the engineered nuclease.

As used herein, “on-target cleavage” refers to a single or double stranded cleavage of a target site at the recognition sequence of the engineered nuclease.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the terms “recognition sequence” or “nuclease recognition sequence” or “recognition site” or “nuclease recognition site” refers to a DNA sequence that is bound and cleaved by a nuclease. A nuclease recognition sequence in a recombinant DNA construct or a self-limiting recombinant virus of the present disclosure may be referred to herein as a “construct recognition sequence” or an “engineered nuclease construct recognition sequence.” For example, a “construct recognition sequence” or an “engineered nuclease construct recognition sequence” may refer to a DNA sequence in a recombinant DNA construct or a self-limiting recombinant virus of the present disclosure that is bound and cleaved by a nuclease, such as a first or second engineered nuclease described herein. Alternatively, a nuclease recognition sequence in the genome or chromosomal DNA of a cell (e.g., a target cell) of the present disclosure may be referred to herein as a “genomic recognition sequence.” For example, a “genomic recognition sequence” may refer to a DNA sequence in the genome or chromosomal DNA of a cell, such as a target cell, that is bound and cleaved by a nuclease, such as a first or second engineered nuclease described herein. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

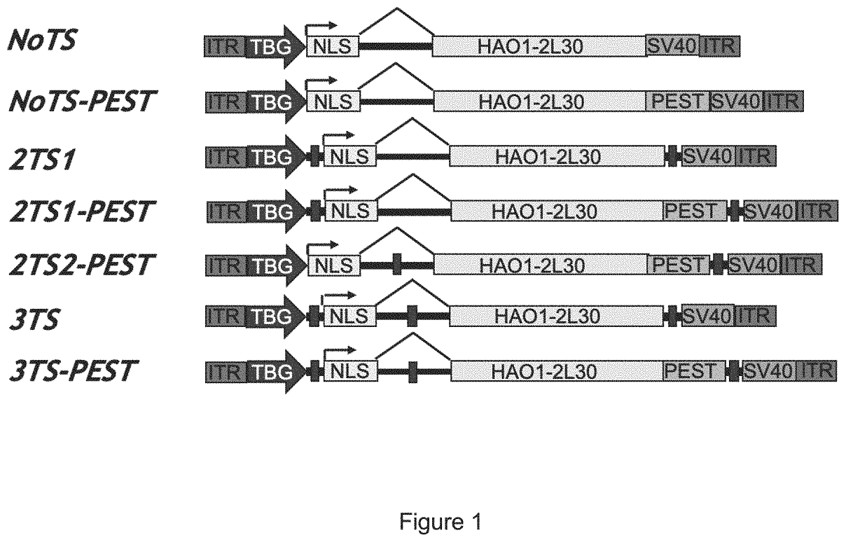

As used herein, the term “recognition sequence” when referring to an engineered I-CreI derived meganuclease refers to a DNA sequence that is bound and cleaved by wild-type I-CreI or an engineered I-CreI-derived meganuclease of the disclosure. The disclosed recognition sequences cleaved by I-CreI and the disclosed engineered meganucleases are typically 22 nucleotides in length. These recognition sequences comprise a pair of inverted, 9 base pair “half-sites” (each numbered from −1 to −9) which are separated by a four base pair center sequence (numbered +1, +2, +3, and +4) (FIG. 1). In the case of a single-chain meganuclease, the N-terminal domain of the protein recognizes, interacts with and/or contacts one half-site and the C-terminal domain of the protein recognizes, interacts with and/or contacts the other half-site. Cleavage by a meganuclease produces four base pair 3′ “overhangs”. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 base pair recognition sequence. Thus, an I-CreI meganuclease recognition sequence may be defined according to formula I:

X₋₉X₋₈X₋₇X₋₆X₋₅X₋₄X₋₃X₋₂X₋₁N₊₁N₊₂N₊₃N₊₄X₋1X₋2X₋₃X₋₄X₋₅X₋₆X₋₇
X₋₈X₋₉,

wherein X and N are each independently nucleotides selected from an adenine nucleotide, a cytosine nucleotide, a guanine nucleotide, and a thymine nucleotide; wherein N₊₁N₊₂N₊₃N₊₄ is the four base pair center sequence.

As used herein, the term “target site” or “target sequence” refers to a region of the chromosomal DNA of a cell comprising a recognition sequence for a site specific nuclease, such as an engineered nuclease described herein. A target site or target sequence can also be located on the recombinant DNA construct or recombinant self-limiting virus disclosed herein.

As used herein, the term “center sequence” refers to the four base pairs separating half-sites in the meganuclease recognition sequence. These bases are numbered +1 through +4. The center sequence comprises the four bases that become the 3′ single-strand overhangs following meganuclease cleavage. “Center sequence” can refer to the sequence of the sense strand or the antisense (opposite) strand. Meganucleases are symmetric and recognize bases equally on both the sense and antisense strand of the center sequence. For example, the sequence A₊₁A₊₂A₊₃A₊₄ on the sense strand is recognized by, interacted with and/or contacted by a meganuclease as T₊₁T₊₂T₊₃T₊₄ on the antisense strand and, thus, A₊₁A₊₂A₊₃A₊₄ and T₊₁T₊₂T₊₃T₊₄ are functionally equivalent (e.g., both can be cleaved by a given meganuclease). Thus, the sequence C₊₁T₊₂G₊₃C₊₄, is equivalent to its opposite strand sequence, G₊₁C₊₂A₊₃G₊₄ due to the fact that the meganuclease binds its recognition sequence as a symmetric homodimer. In most cases, a first subunit of the meganuclease recognizes, interacts with and/or contacts the first two base pairs of the sense strand of a given center sequence and the second two base pairs on the antisense. For example, taking A₊₁A₊₂A₊₃A₊₄ as the center sequence, a first subunit would recognize, interact with and/or contact the two base pairs A₊₁A₊₂, and a second subunit would recognize, interact with and/or contact the anti-sense strand two base pairs A₊₃A₊₄ on the anti-sense strand, which is T₊₄T₊₃.

As used herein, the term “center sequence half-site,” or simply “center half-site” refers to either the 5′ two base pairs or the 3′ two base pairs of a four base pair center sequence of a recognition sequence as described herein. For example, for the center sequence ACAG, the 5′ two base pairs (i.e., the 5′ center half site) of the center sequence is “AC” and the 3′ two base pairs (i.e., the 3′ center half site) is “AG” (reverse complement being “CT”).

As used herein, the term “homologous recombination” or “HR” refers to the natural, cellular process in which a double-stranded DNA-break is repaired using a homologous DNA sequence as the repair template (see, e.g. Cahill et al. (2006) Front. Biosci. 11:1958-1976). The homologous DNA sequence may be an endogenous chromosomal sequence or an exogenous nucleic acid that was delivered to the cell. The term “homology” is used herein as equivalent to “sequence similarity” and is not intended to require identity by descent or phylogenetic relatedness.

As used herein, the term “homology arms” or “sequences homologous to sequences flanking a nuclease cleavage site” refer to sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term “non-homologous end-joining” or “NHEJ” refers to the natural, cellular process in which a double-stranded DNA-break is repaired by the direct joining of two non-homologous DNA segments (see, i.e. Cahill et al. (2006) Front. Biosci. 11:1958-1976). DNA repair by non-homologous end-joining is error-prone and frequently results in the untemplated addition or deletion of DNA sequences at the site of repair. The process of non-homologous end-joining occurs in both eukaryotes and prokaryotes such as bacteria.

As used herein, the term “re-ligation” refers to a process in which two DNA ends produced by a pair of double-strand DNA breaks are covalently attached to one another with the loss of the intervening DNA sequence but without the gain or loss of any additional DNA sequence. In the case of a pair of DNA breaks produced with single-strand overhangs, re-ligation can proceed via annealing of complementary overhangs followed by covalent attachment of 5′ and 3′ ends by a DNA ligase. Re-ligation is distinguished from NHEJ in that it does not result in the untemplated addition or removal of DNA from the site of repair.

As used herein, the term “concatemer” refers to long continuous DNA molecules that contain multiple copies of the same DNA sequence linked in series,

As used herein, the term “persistence” or “persist” refers to the viability of the self-limiting recombinant virus in the cell, tissue, or organism of interest. Attenuating persistence time refers to the degradation of the recombinant virus, and thus, viral genome.

As used herein, the term “promoter” or “regulatory sequence” refers to a nucleic acid sequence which is required for expression of a gene product operably linked to the promoter or regulatory sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in a tissue-specific manner, a species-specific manner, an inducible manner, and/or a constitutive manner.

As used herein, the term “constitutive promoter” refers to a nucleotide sequence which, when operably linked with a polynucleotide which encodes or specifies a gene product, causes the gene product to be produced in a cell under most or all physiological conditions of the cell.

As used herein, the term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, the term “operably linked” is intended to mean a functional linkage between two or more elements. For example, an operable linkage between a promoter and a nucleic acid sequence encoding an engineered nuclease as disclosed herein is a functional link that allows for expression of the nucleic acid sequence encoding the engineered nuclease. Operably linked elements may be contiguous or non-contiguous. When used to refer to the joining of two protein coding regions, by operably linked is intended that the coding regions are in the same reading frame.

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

As used herein, the terms “transfected” or “transformed” or “transduced” or “nucleofected” refer to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, persistence time refers to the amount of time that the self-limiting recombinant virus or recombinant DNA construct is present in the cell and able to encode the production of the engineered nuclease.

As used herein with respect to a parameter, the term “increased” or “increasing” or “increase” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) positive change in the parameter from a comparison control, e.g., an established normal or reference level of the parameter, or an established standard control. For example, increased on-target cleavage of the genome of the target cell by a self-limiting recombinant virus may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) increase or positive change in on-target cleavage of the genome of the target cell by the self-limiting recombinant virus compared to on-target cleavage of the genome of the target cell by a control recombinant virus.

As used herein with respect to a parameter, the term “decreased” or “decreasing” or “decrease” or “reduced” or “reducing” or “reduction” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) negative change in the parameter from a comparison control, e.g., an established normal or reference level of the parameter, or an established standard control. For example, decreased off-target cleavage of the genome of the target cell by a self-limiting recombinant virus may indicate detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) decrease or negative change in off-target cleavage of the genome of the target cell by the self-limiting recombinant virus compared to on-target cleavage of the genome of the target cell by a control recombinant virus.

As used herein, a “control recombinant virus” refers to a virus, such as a recombinant virus that has not been subject to the methods and compositions described herein. For example, a control recombinant virus may refer to a recombinant virus that is not encoded by a recombinant DNA construct described herein, such as a recombinant virus that does not contain the two or more nuclease recognition sequences cleaved by the engineered nuclease. In some embodiments, a control recombinant virus may refer to a recombinant virus that does not contain the two or more nuclease recognition sequences cleaved by the engineered nuclease but which may otherwise be similar to the self-inactivating recombinant virus of the present disclosure.

As used herein, “a,” “an,” or “the” can mean one or more than one. For example, “an” endonuclease can mean a single endonuclease or a multiplicity of endonucleases. Further, the term “a gene” may include a plurality of genes, including a group of several genes.

As used herein, unless specifically indicated otherwise, the word “or” is used in the inclusive sense of “and/or” and not the exclusive sense of “either/or.”

As used herein, the term “about” or “approximately” usually means within 5%, or more preferably within 1%, of a given value or range.

As used herein, the terms “comprises”, “comprising”, “includes”, “including”, “having” and their conjugates mean “including but not limited to”.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values and if the variable is inherently continuous. As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### 2.1. Principle of the Invention

Significant advances in engineering sequence specific nucleases have enabled a broad range of biomedical applications, particularly when combined with recombinant adeno-associated virus (AAV), a versatile viral vector for in vivo post-mitotic cell gene delivery. The long-term expression of nuclease mediated by AAV delivery, however, raises concerns about specificity and immunogenicity. Persistent expression of the nuclease can increase the likelihood of off-target cleavage which can induce genotoxicity. Moreover, expression of an exogenous nuclease has the potential to elicit an immune response against transduced cells. Thus, there is an unmet need to limit the duration of nuclease expression following the AAV delivery.

The present disclosure provides recombinant DNA constructs that are self-inactivating through the use of engineered nuclease technology. In some embodiments, the recombinant DNA constructs described herein encode a self-inactivating AAV system using engineered nuclease technology (e.g., using engineered meganucleases). The self-inactivating AAV system of the present disclosure can contain a tissue-specific promoter, a nuclease open reading frame (ORF), and adjacent sites targeted by the nuclease (i.e., one or more nuclease construct recognition sequences). Data disclosed herein demonstrate in vitro that this system can progressively reduce nuclease expression over time, and the decrease in nuclease expression can be impacted by the locations and copy numbers of the target site insertions. Furthermore, data provided herein demonstrated in vivo that this AAV system can eliminate ˜80% of nuclease expression within 6 weeks in mouse liver, while enabling ˜70% of on-target cleavage efficiency. In addition, off-target cutting and AAV insertions and deletions (indels) were measured to fully evaluate the efficiency of the system. Overall, the self-inactivating AAV system of the present disclosure has the potential to improve therapeutic applications that use engineered nucleases.

### 2.2. Recombinant DNA Construct Encoding Self-Limiting Recombinant Virus

Disclosed herein are recombinant DNA constructs encoding an engineered nuclease with target sites of the nuclease located in specific positions on the recombinant construct in order to limit the persistence of the construct in a cell when the nuclease is expressed. Also described herein are plasmids containing the recombinant DNA constructs of the present disclosure, along with recombinant viruses containing the recombinant DNA constructs of the present disclosure. In certain embodiments, the recombinant virus is a recombinant adenovirus, a recombinant lentivirus, a recombinant retrovirus, or a recombinant adeno-associated virus (AAV). In particular, the recombinant virus of the present disclosure is a recombinant AAV (rAAV). In specific embodiments, the recombinant AAV of the present disclosure has an AAV8 serotype. Alternatively, the recombinant AAV of the present disclosure may have an AAV5 serotype. In yet other embodiments, the recombinant AAV of the present disclosure may have an AAV2 serotype.

A viral vector is sometimes referred to herein as a recombinant virus. For example, an AAV vector is often referred to herein as a recombinant AAV. Similarly, a self-limiting viral vector of the present disclosure is often referred to herein as a self-limiting recombinant virus. For example, a self-limiting AAV vector is often referred to herein as a self-limiting recombinant AAV.

The present disclosure is based, in part, on the premise that a recombinant virus, such as a recombinant AAV, will not persist in a cell after cleavage of the DNA by a nuclease. Recombinant AAV is a preferred vector for delivery of genome editing nucleases to cells and tissues, but its long persistence time in cells often presents a problem. Genome editing applications using site-specific nucleases generally do not require long-term expression of the nuclease gene, and long-term expression of nuclease may even be harmful. Long-term expression of nucleases may hinder cleavage specificity, thus introducing breakage in unintended sites, which may lead to detrimental consequences for cell health. Moreover, cell machinery is designed to detect and immunologically respond to the production of foreign proteins, such as nucleases introduced by a recombinant AAV (Mingozzi and High, Blood 122:23-26 (2013)). Thus, the present disclosure provides recombinant DNA constructs that encode a recombinant virus, wherein the persistence time of the recombinant virus is “self-limited” through a recognition sequence for the genome editing nuclease already incorporated into the recombinant virus.

The self-limiting recombinant virus is thus able to deliver the nuclease gene to a cell or tissue such that the nuclease is expressed and able to modify the genome of the cell. In addition, the same nuclease will find its target site within the recombinant virus and will cut the genome of the virus, exposing free 5′ and 3′ ends and initiating degradation by exonucleases. Cleavage of the viral genome will prevent the virus from forming concatemers that can persist stably in the cell as episomes. Thus, the virus effectively “kills itself.”

A self-limiting recombinant virus of the present disclosure can be encoded by a recombinant DNA construct, such as a DNA construct that contains: a polynucleotide that encompasses a nucleic acid sequence encoding an engineered nuclease; a promoter that is operably linked to the nucleic acid sequence encoding the engineered nuclease and drives expression of the engineered nuclease in a target cell; and two or more engineered nuclease construct recognition sequences that are bound and cleaved by the engineered nuclease.

### 2.2.1. Polynucleotide Encoding Engineered Nuclease

The recombinant DNA constructs disclosed herein incorporate a polynucleotide comprising a nucleic acid sequence encoding an engineered nuclease. In specific embodiments, a self-limiting recombinant virus of the present disclosure can be encoded by a recombinant DNA construct that contains a polynucleotide, which encompasses a nucleic acid sequence encoding an engineered nuclease.

In some embodiments, the polynucleotide contains a nuclear localization sequence (NLS) attached to the nucleic acid sequence encoding the engineered nuclease. The NLS may facilitate nuclear transport of the engineered nuclease after the nuclease is expressed in a target cell. In some embodiments, the polynucleotide may contain SV40 NLS, which is the NLS of SV40 large T antigen. In some instances, the NLS is positioned 5′ upstream of the nucleic acid sequence encoding the engineered nuclease. For example, the NLS may be positioned 10-500 (e.g., 15-450, 20-400, 25-350, 30-300, 35-250, 40-200, 45-150, or 50-100) nucleotides, such as about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 nucleotides upstream of the nucleic acid sequence encoding the engineered nuclease. In some instances, the NLS is positioned 3′ downstream of the nucleic acid sequence encoding the engineered nuclease. For example, the NLS may be positioned 10-500 (e.g., 15-450, 20-400, 25-350, 30-300, 35-250, 40-200, 45-150, or 50-100) nucleotides, such as about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 nucleotides downstream of the nucleic acid sequence encoding the engineered nuclease.

In some embodiments, the polynucleotide contains an intron that is inserted in the nucleic acid sequence encoding the engineered nuclease. In certain embodiments, a mammalian intron, such as the human growth hormone (HGH) intron (SEQ ID NO: 36), or the SV40 large T antigen intron (SEQ ID NO: 37) may be inserted in the nucleic acid sequence encoding the engineered nuclease. The intron may be inserted in the nuclease open reading frame (ORF) to restrict the expression of the nuclease in a non-target cell, such as in a non-mammalian cell. In some instances, the intron may be positioned 3′ downstream of the NLS. For example, the intron may be positioned 1-250 (e.g., 5-250, 10-200, 15-150, 20-100, or 25-50) nucleotides, such as about 1, 5, 10, 15, 20, 25, 30, 35, 40, 45, 50, 55, 60, 65, 70, 75, 80, 85, 90, 95, 100, 125, 150, 175, 200, 225, or 250 nucleotides downstream of the nucleic acid sequence encoding the engineered nuclease. In additional or alternative instances, the intron may be positioned 5′ upstream of an exon of the nucleic acid sequence encoding the engineered nuclease. For example, the intron may be positioned 1-250 (e.g., 5-250, 10-200, 15-150, 20-100, or 25-50) nucleotides, such as about 1, 5, 10, 15, 20, 25, 30, 35, 40, 45, 50, 55, 60, 65, 70, 75, 80, 85, 90, 95, 100, 125, 150, 175, 200, 225, or 250 nucleotides upstream of an exon of the nucleic acid sequence encoding the engineered nuclease.

In some embodiments, a self-limiting recombinant virus of the present disclosure can be encoded by a recombinant DNA construct that contains a polyadenylation signal sequence (polyA sequence or polyA tail), which is attached downstream of the nucleic acid sequence encoding the engineered nuclease. In certain embodiments, a polyA tail attached downstream of the nucleic acid sequence encoding the engineered nuclease can add a polyadenylation tail to the nuclease mRNA and terminate transcription of the nuclease. In particular, a SV40 polyadenylation signal sequence (SV40 polyA) can be attached 3′ downstream of the nucleic acid sequence encoding the engineered nuclease.

In some embodiments, the recombinant DNA construct or self-limiting recombinant virus of the present disclosure contains one or more (e.g., one, two, three, four, five, six, seven, eight, nine, ten, or more) accessory nucleic acid sequences encoding one or more (e.g., one, two, three, four, five, six, seven, eight, nine, ten, or more) accessory engineered nucleases. For example, a recombinant DNA construct or self-limiting recombinant virus of the present disclosure may contain a second, a third, a fourth, a fifth, a sixth, a seventh, an eighth, a ninth, a tenth, or more nucleic acid sequences encoding a second, a third, a fourth, a fifth, a sixth, a seventh, an eighth, a ninth, a tenth, or more engineered nuclease.

It is known in the art that it is possible to use a site-specific nuclease to make a DNA break in the genome of a living cell, and that such a DNA break can result in permanent modification of the genome via homologous recombination with a transgenic DNA sequence. The use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus.

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus. Such exogenous nucleic acids can encode any sequence or polypeptide of interest.

Thus, in different embodiments, a variety of different types of nucleases are useful for practicing the invention. In one embodiment, the invention can be practiced using engineered recombinant meganucleases. In another embodiment, the invention can be practiced using a CRISPR system nuclease or CRISPR system nickase. Methods for making CRISPR and CRISPR Nickase systems that recognize and bind pre-determined DNA sites are known in the art, for example Ran, et al. (2013) Nat Protoc. 8:2281-308. In another embodiment, the invention can be practiced using TALENs or Compact TALENs. Methods for making TALE domains that bind to pre-determined DNA sites are known in the art, for example Reyon et al. (2012) Nat Biotechnol. 30:460-5. In another embodiment, the invention can be practiced using zinc finger nucleases (ZFNs). In a further embodiment, the invention can be practiced using megaTALs.

Engineered nuclease and accessory engineered nucleases of the present disclosure can be one or more (e.g., one, two, three, four, five, six, seven, eight, nine, ten, or more) of an engineered meganuclease, an engineered TALEN, an engineered compact TALEN, an engineered zinc finger nuclease, an engineered CRISPR/Cas9 nuclease, or an engineered megaTAL. In particular, an engineered nuclease of the present disclosure can be an engineered meganuclease. The accessory engineered nucleases can be any engineered nuclease disclosed here and need not necessarily be the same engineered nuclease or class of engineered nuclease as the other engineered nuclease encoded by the recombinant DNA construct. For example, all engineered nucleases encoded by the recombinant DNA construct or recombinant virus disclosed herein could be engineered meganucleases or could be a mix of an engineered meganuclease and a engineered TALEN, an engineered compact TALEN, an engineered zinc finger nuclease, an engineered CRISPR/Cas9 nuclease, or an engineered megaTAL.

In some embodiments, the engineered meganuclease is an engineered meganuclease as published in the of any of International Publication Nos. WO2007/047859, WO2009059195, WO2010/009147, WO2012/167192, WO2015/138739, WO2016/179112, WO2017/044649, WO2017/062439, WO2017/062451, WO2017/112859, WO2017/192741, WO2018/071849, WO2018/195449, WO2019/005957, WO2019/089913, WO2019/200122, and WO2019/200247, and International Publication Nos. PCT/US2019/068186 and PCT/US2020/013198, each of which is incorporated by reference in its entirety herein. In some embodiments, an “I-CreI-derived meganuclease” specifically includes any engineered meganuclease within the scope of the issued claims of any of U.S. Pat. Nos. 8,021,867, 8,119,361, 8,119,381, 8,124,369, 8,129,134, 8,133,697, 8,143,015, 8,143,016, 8,148,098, 8,163,514, 8,304,222, 8,377,674, 8,445,251, 9,340,777, 9,434,931, 10,041,053, 9,683,257, 10,287,626, 10,273,524, 9,683,257, 10,287,626, 10,273,524, 9,822,381, 10,603,363, 9,889,160, 9,889,161, 9,993,501, 9,993,502, 9,950,010, 9,950,011, 9,969,975, 10,093,899, and 10,093,900, each of which is incorporated by reference herein. In some embodiments, an engineered I-CreI-derived meganuclease comprises a polypeptide having at least 85% sequence identity to residues 2-153 of the I-CreI meganuclease of SEQ ID NO: 1, as in the issued claims of each of U.S. Pat. Nos. 8,021,867, 8,119,361, 8,119,381, 8,124,369, 8,129,134, 8,133,697, 8,143,015, 8,143,016, 8,148,098, 8,163,514, 8,304,222, 8,377,674. In some embodiments, an engineered I-CreI-derived meganuclease comprises a polypeptide having at least 86%, 87%, 88%, 89%, 90%, 91%, 92%, 93%, 94%, 95%, 96%, 97%, 98% or 99% sequence identity to residues 2-153 of the I-CreI meganuclease of SEQ ID NO: 1.

### 2.2.2. Promoter Driving Expression of Engineered Nuclease

A recombinant DNA construct and self-limiting recombinant virus of the present disclosure can contain a promoter, which is operably linked to the nucleic acid sequence encoding the engineered nuclease. In some embodiments, the promoter is positioned 5′ upstream of the nucleic acid sequence and drives expression of the engineered nuclease in a target cell. In particular embodiments, the promoter is positioned 5′ upstream of the NLS.

As described hereinabove, in some embodiments, a recombinant DNA construct and self-limiting recombinant virus of the present disclosure can contain one or more accessory nucleic acid sequences encoding one or more accessory engineered nucleases. In some such embodiments, the recombinant DNA construct may contain: (i) a first promoter, which is operably linked to the nucleic acid sequence (such as a first nucleic acid sequence) encoding the engineered nuclease (such as a first engineered nuclease), and drives the expression of the engineered nuclease in a target cell; and (ii) one or more accessory promoters, which are operably linked to the one or more accessory nucleic acid sequences encoding one or more accessory engineered nucleases, and drive the expression of the one or more accessory engineered nucleases in a target cell. Alternatively, some such recombinant DNA constructs may contain a single promoter, which drives the expression of the engineered (such as the first engineered nuclease) and the one or more accessory engineered nuclease.

A promoter for use in the compositions and methods described herein can be one or more of a tissue-specific promoter, a species-specific promoter, an inducible promoter, and/or a constitutive promoter.

Tissue-Specific Promoter

In some embodiments, a recombinant DNA construct and self-limiting recombinant virus of the present disclosure can contain a tissue-specific promoter. For example, a recombinant DNA construct described herein may contain a tissue-specific promoter that is operably linked to the nucleic acid sequence encoding the engineered nuclease. A tissue-specific promoter may drive expression of the engineered nuclease in that specific tissue. In certain embodiments, a tissue-specific promoter for use in the compositions and methods described herein may be a liver-specific promoter. A liver-specific promoter may drive expression of the engineered nuclease specifically in liver, and not in any other tissues. Examples of liver-specific promoters include, but are not limited to, albumin promoters (such as Palb), human al-antitrypsin (such as Pa1AT), and hemopexin (such as Phpx) (Kramer et al., Mol Therapy 7:375-85 (2003)). In particular, liver-specific promoters for use in the compositions and methods described herein may be one or more of a human thyroxine binding globulin (TBG) promoter, a human alpha-1 antitrypsin promoter, a hybrid liver specific promoter, or an apolipoprotein A-II promoter. Additionally, or alternatively, a tissue-specific promoter for use in the compositions and methods described herein may be an ocular-specific or eye-specific promoter. An ocular-specific or eye-specific promoter may drive expression of the engineered nuclease specifically in eye, and not in any other tissues. Examples of ocular-specific or eye-specific promoters include, but are not limited to opsin, and corneal epithelium-specific K12 promoters (Martin et al., Methods 28:267-75 (2002); Tong et al., J Gene Med 9:956-66 (2007)) and human G-protein-coupled receptor protein kinase 1 (GRK1) promoter. Additionally, or alternatively, a tissue-specific promoter for use in the compositions and methods described herein may be a muscle-specific promoter. A muscle-specific promoter may drive expression of the engineered nuclease specifically in muscles, and not in any other tissues. Examples of muscle-specific promoters include, but are not limited to C5-12 (Liu et al., Hum Gene Ther 15:783-92 (2004)), the muscle-specific creatine kinase (MCK) promoter (Yuasa et al., Gene Ther 9:1576-88 (2002)), or the smooth muscle 22 (SM22) promoter (Haase et al., BMC Biotechnol 13:49-54 (2013)). Additionally, or alternatively, a tissue-specific promoter for use in the compositions and methods described herein may be a central nervous system (CNS)-specific or neuron-specific promoter. A (CNS)-specific or neuron-specific promoter may drive expression of the engineered nuclease specifically in the CNS, such as in neurons, and not in any other tissues. Examples of CNS (neuron)-specific promoters include, but are not limited to NSE, Synapsin, and MeCP2 promoters (Lentz et al., Neurobiol Dis 48:179-88 (2012)). Other non-limiting examples of tissue specific promoters for use in the compositions and methods described herein include: synovial sarcomas PDZD4 (specific to cerebellum); C6 (specific to liver); cholesterol regulation APOM (specific to liver); ASB5 (specific to muscle); monogenic malformation syndromes TP73L (specific to muscle); SLC5A12 (specific to kidney); PPP1R12B (specific to heart); and ADPRHL1 (specific to heart) (Jacox et al., PLoS One v.5(8):e12274 (2010)).

Species-Specific Promoter

In some embodiments, a recombinant DNA construct and self-limiting recombinant virus of the present disclosure can contain a species-specific promoter. For example, a recombinant DNA construct described herein may contain a species-specific promoter that is operably linked to the nucleic acid sequence encoding the engineered nuclease. A species-specific promoter may drive expression of the engineered nuclease in that specific species. In certain embodiments, a tissue-specific promoter for use in the compositions and methods described herein may be a mammalian promoter. A mammalian promoter may drive expression of the engineered nuclease specifically in mammalian cells, and not in non-mammalian cells. Examples of mammalian promoters include, but are not limited to cytomegalovirus- or SV40 virus-early promoters.

Inducible Promoter

In some embodiments, a recombinant DNA construct and self-limiting recombinant virus of the present disclosure can contain an inducible promoter. For example, a recombinant DNA construct described herein may contain an inducible promoter that is operably linked to the nucleic acid sequence encoding the engineered nuclease. In some such embodiments, the recombinant DNA construct further contains a nucleic acid sequence encoding a ligand-inducible transcription factor, wherein the ligand-inducible transcription factor regulates activation of the inducible promoter, and eventually, expression of the nuclease. In certain embodiments, a small-molecule inducer may be required for activation of the inducible promoter and expression of the nuclease. Examples of inducible promoters include, but are not limited to Tet-On system (Clontech; Chen et al., BMC Biotechnol 15(1):4 (2015)) and the RheoSwitch system (Intrexon; Sowa et al., Spine 36(10): E623-8 (2011)). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). The transcription activator can induce gene expression of the engineered nuclease only in cells or tissues that are treated with the cognate small-molecule activator. Use of such inducible promoters is advantageous because it enables gene expression of the engineered nuclease to be regulated in a spatio-temporal manner by selecting when and to which tissues the small-molecule inducer is delivered.

Constitutive Promoter

In some embodiments, a recombinant DNA construct and self-limiting recombinant virus of the present disclosure can contain a constitutive promoter. For example, a recombinant DNA construct described herein may contain a constitutive promoter that is operably linked to the nucleic acid sequence encoding the engineered nuclease. In certain embodiments, a constitutive promoter for use in the compositions and methods described herein may be a native promoter. Additionally, or alternatively, a constitutive promoter for use in the compositions and methods described herein may be a composite promoter.

### 2.2.3. Nuclease Recognition Sequence

A recombinant DNA construct and self-limiting recombinant virus of the present disclosure can contain two or more (e.g., two, three, four, five, six, seven, eight, nine, ten, or more) nuclease recognition sequences, which are bound and cleaved by the engineered nuclease. A nuclease recognition sequence in a recombinant DNA construct or a self-limiting recombinant virus of the present disclosure may be referred to herein as a construct recognition sequence or an engineered nuclease construct recognition sequence. For example, a DNA sequence in a recombinant DNA construct or a self-limiting recombinant virus of the present disclosure that is bound and cleaved by a nuclease, such as a first or second engineered nuclease may be referred to herein as a construct recognition sequence or engineered nuclease construct recognition sequence. Alternatively, a nuclease recognition sequence in the genome or chromosomal DNA of a cell (e.g., a target cell) of the present disclosure may be referred to herein as a genomic recognition sequence. For example, a DNA sequence in the genome or chromosomal DNA of a cell, such as a target cell, that is bound and cleaved by a nuclease, such as a first or second engineered nuclease of the present disclosure may be referred to herein as a genomic recognition sequence.

In certain embodiments, a recombinant DNA construct and self-limiting recombinant virus of the present disclosure contains two nuclease recognition sequences, such as a first nuclease recognition sequence and a second nuclease recognition sequences. In some such embodiments, the distance between the first and the second nuclease recognition sequences is at least 500 (e.g., at least 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides. In other embodiments, the distance between the first and the second nuclease recognition sequences is about 500-2500 nucleotides, such as about 600-2500, 700-2500, 800-2500, 900-2500, or 1000-2500 (e.g., about 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides. In certain embodiments, a self-limiting recombinant virus of the present disclosure is encoded by a recombinant DNA construct that contains three nuclease recognition sequences, such as a first nuclease recognition sequence, a second nuclease recognition sequence, and a third nuclease recognition sequence. In some such embodiments, the distance between the first and the second nuclease recognition sequences is at least 50 (e.g., at least 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides and the distance between the second and the third nuclease recognition sequences is at least 500 (e.g., at least 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, or more) nucleotides. In other embodiments, the distance between the first and the second nuclease recognition sequences is about 50-2500 (e.g., about 50-75, 75-100, 100-150, 150-200, 200-250, 250-300, 300-350, 350-400, 400-450, 450-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides and the distance between the second and the third nuclease recognition sequences is about 500-2500 nucleotides, such as about 600-2500, 700-2500, 800-2500, 900-2500, or 1000-2500 (e.g., about 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, or 2400-2500) nucleotides.

In some embodiments, a recombinant DNA construct of the present disclosure contains two or more nuclease recognition sequences, of which at least one (e.g., at least one, two, three, four, five, six, seven, eight, nine, ten, or more) nuclease recognition sequence may be positioned 3′ downstream of the intron described hereinabove. For example, at least one of the two or more nuclease recognition sequences may be positioned 3′ downstream of a mammalian intron, such as a human growth hormone (HGH) intron, or a SV40 large T antigen intron. In additional or alternative embodiments, a recombinant DNA construct of the present disclosure contains two or more nuclease recognition sequences, of which at least one (e.g., at least one, two, three, four, five or more) nuclease recognition sequence may be positioned 5′ upstream of the intron described hereinabove. For example, at least one of the two or more nuclease recognition sequences may be positioned 5′ upstream of a mammalian intron, such as a human growth hormone (HGH) intron, or a SV40 large T antigen intron. In additional or alternative embodiments, a recombinant DNA construct of the present disclosure contains two or more nuclease recognition sequences, of which at least one nuclease recognition sequence may be positioned within the intron described hereinabove. For example, at least one of the two or more nuclease recognition sequences may be positioned within a mammalian intron, such as a human growth hormone (HGH) intron, or a SV40 large T antigen intron.

In some embodiments, the two or more nuclease recognition sequences are identical. For example, a recombinant DNA construct of the present disclosure may contain two or more identical nuclease recognition sequences. In additional or alternative embodiments, the two or more nuclease recognition sequences are non-identical. For example, a recombinant DNA construct of the present disclosure may contain two or more non-identical nuclease recognition sequences. In certain embodiments, the two or more non-identical nuclease recognition sequences can be recognized by the same engineered nuclease, such as a first engineered nuclease, and one or more accessory engineered nucleases. For example, a self-limiting recombinant virus of the present disclosure can be encoded by a recombinant DNA construct that contains one or more accessory nucleic acid sequences encoding one or more accessory engineered nucleases; and in such embodiments, the two or more non-identical nuclease recognition sequences can be recognized by an engineered nuclease, such as a first engineered nuclease, and one or more accessory engineered nucleases. In certain embodiments, the non-identical nuclease recognition sequences can differ by at least 1 nucleic acid, such as by 1, 2, 3, 4, 5, 6, 7, 8, 9, 10, 11, or 12 nucleic acids.

In some embodiments, the engineered nuclease binds and cleaves a genomic recognition sequence in the genome of a target cell. For example, following expression in a target cell, the engineered nuclease may bind and cleave a genomic recognition sequence in the genome of a target cell. In certain embodiments, the genomic recognition sequence is identical to the two or more nuclease recognition sequences in the recombinant DNA construct. In other embodiments, the genomic recognition sequence is not identical to the two or more nuclease recognition sequences in the recombinant DNA construct. In some such embodiments, the genomic recognition sequence and the two or more nuclease recognition sequences in the recombinant DNA construct contain different recognition sequences. In some embodiments, the genomic recognition sequence and the two or more nuclease recognition sequences in the recombinant DNA construct contain different recognition sequences for different types of engineered nucleases. In some embodiments, the genomic recognition sequence and at least one of the two or more nuclease recognition sequences in the recombinant DNA are engineered meganuclease recognition sequences.

It has been recently described that the cleavage of a recognition sequence within an engineered meganuclease can be enhanced by altering certain center sequence interacting positions corresponding to positions 48, 50, 71, 72, 73, 73B, and 74 of an I-CreI derived engineered meganuclease as described in PCT/US2020/31879, which is incorporated by reference herein. Additional center sequences for engineered meganucleases are described in PCT/US2009/050566, which is incorporated by reference herein. Thus, in some such embodiments, the genomic recognition sequence and the two or more nuclease recognition sequences in the recombinant DNA construct contain different center sequences but identical half-site sequences. In some embodiments, the center sequence of the engineered meganuclease is a four base pair center sequence comprising ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, TTAA, GTAA, GTAG, GTAT, GTGA, GTGC, GTGG, or GTGT. In some embodiments, the center sequence of the engineered meganuclease is a four base pair center sequence comprising TTGT, TTAT, TCTT, TCGT, TCAT, GTTT, GTCT, GGAT, GAGT, GAAT, ATGT, TTTC, TTCC, TGAC, TAAC, GTTC, ATAT, TCGA, TTAA, GGGC, ACGC, CCGC, CTGC, ACAA, ATAA, AAGA, ACGA, ATGA, AAAC, AGAC, ATCC, ACTC, ATTC, ACAT, GAAA, GGAA, GTCA, GTTA, GAAC, ATAT, TCGA, TTAA, GCCC, GCGT, GCGG or GCAG. In some embodiments, the center sequence of the engineered meganuclease is a four base pair center sequence comprising GTGT, GTAT, TTAG, GTAG, TTAC, TCTC, TCAC, GTCC, GTAC, TCGC, AAGC, GAGC, GCGC, GTGC, TAGC, TTGC, ATGC, ACAC, ATAC, CTAA, CTAC, GTAA, GAGA, GTGA, GGAC, GTAC, GCGA, GCTT, GCTC, GCGC, GCAC, GCTA, GCAA or GCAT. Additionally, in some such embodiments, a cleavage rate of the engineered nuclease for at least one of the two or more nuclease recognition sequences in the recombinant DNA construct may be about 50-90% (e.g., about 55-90%, 60-90%, 65-90%, 70-90%, 75-90%, 80-90%, or 85-90%), such as about 50%, 55%, 60%, 65%, 70%, 75%, 80%, 85%, 86%, 87%, 88%, 89%, or 90% of the cleavage rate of the engineered nuclease for the genomic recognition sequence. In additional or alternative embodiments, an engineered nuclease may not substantially cleave the two or more construct recognition sequences in the recombinant DNA construct.

As described herein above, a recombinant DNA construct or self-limiting recombinant virus of the present disclosure may contain one or more accessory nucleic acid sequences encoding one or more accessory engineered nucleases (e.g., a second engineered nuclease). In some embodiments, cleavage rate of the accessory engineered nuclease for the genomic recognition sequence may be about 50-90% (e.g., about 55-90%, 60-90%, 65-90%, 70-90%, 75-90%, 80-90%, or 85-90%), such as about 50%, 55%, 60%, 65%, 70%, 75%, 80%, 85%, 86%, 87%, 88%, 89%, or 90% of the cleavage rate of the accessory engineered nuclease for at least one of the two or more nuclease recognition sequences in the recombinant DNA construct. For example, cleavage rate of a second engineered nuclease for the genomic recognition sequence may be about 50-90% (e.g., about 55-90%, 60-90%, 65-90%, 70-90%, 75-90%, 80-90%, or 85-90%), such as about 50%, 55%, 60%, 65%, 70%, 75%, 80%, 85%, 86%, 87%, 88%, 89%, or 90% of the cleavage rate of the second engineered nuclease for at least one of the two or more nuclease recognition sequences in the recombinant DNA construct.

The precise location of the nuclease recognition sequence in the recombinant DNA construct may vary, as exemplified in FIG. 1. One exemplified configuration comprises at least one nuclease recognition sequence positioned within an intron in the nucleic acid sequence encoding the engineered nuclease gene (see, for example, 2TS2-PEST, 3TS, and 3TS-PEST in FIG. 1). Intracellular cleavage of a recombinant virus that is encoded by such a recombinant DNA construct is expected to separate the two exons of the engineered nuclease gene such that the engineered nuclease gene can no longer be expressed. This leads to a more rapid attenuation of expression of the engineered nuclease. Alternatively, it is possible to position at least one nuclease recognition sequence between the engineered nuclease gene and its promoter, i.e., in the 5′ UTR (see, for example, 2TS1, 2TS1-PEST, 3TS, and 3TS-PEST in FIG. 1). This configuration can also quickly attenuate expression of the engineered nuclease while not adding as much additional size to the gene. Also, at least one nuclease recognition sequence may be placed 3′ downstream of the engineered nuclease gene sequence and 5′ upstream of the polyA sequence (see, for example, 2TS1, 2TS1-PEST, 2TS2-PEST, 3TS, and 3TS-PEST in FIG. 1). The nuclease recognition sequence may be placed in various locations, as long as the site does not interfere with the proper expression of the engineered nuclease, and is accessible by the expressed engineered nuclease.

### 2.2.4. Protein Degradation Peptide

The recombinant DNA constructs and a self-limiting recombinant viruses of the present disclosure may contain a nucleic acid sequence encoding a protein degradation peptide. A nucleic acid sequence encoding a protein degradation peptide is also referred to herein as a protein degradation peptide encoding sequence. Presence of a protein degradation peptide on an engineered nuclease may render the nuclease more susceptible to intracellular proteolysis and may reduce the expression level of the nuclease. Moreover, presence of a protein degradation peptide on an engineered nuclease may also reduce off-target activity of the engineered nuclease. In some embodiments, the protein degradation peptide has a protein sequence that is rich in proline, glutamic acid, serine and threonine. In such embodiments, the protein degradation peptide is referred to herein as a PEST sequence. Identification of PEST amino acid sequences or “PEST sequences” is well known in the art, and is described, for example in Rogers S et al (Amino acid sequences common to rapidly degraded proteins: the PEST hypothesis. Science 1986; 234(4774):364-8) and Rechsteiner M et al (PEST sequences and regulation by proteolysis. Trends Biochem Sci 1996; 21(7):267-71). “PEST sequence” refers, in another embodiment, to a region rich in proline (P), glutamic acid (E), serine (S), and threonine (T) residues. In another embodiment, the PEST sequence is flanked by one or more clusters containing several positively charged amino acids. The PEST sequence can mediate intracellular degradation of proteins containing it. In another embodiment, the PEST sequence fits an algorithm disclosed in Rogers et al. In another embodiment, the PEST sequence fits an algorithm disclosed in Rechsteiner et al. In another embodiment, the PEST sequence contains one or more internal phosphorylation sites, and phosphorylation at these sites precedes protein degradation. Alternatively, a protein degradation peptide may have a sequence encoding an intracellular protein degradation signal or degron or ubiquitin sequence.

In some embodiments, the protein degradation peptide encoding sequence is positioned 3′ downstream of the nucleic acid sequence that encodes the engineered nuclease. In additional or alternative embodiments, the protein degradation peptide encoding sequence is positioned 5′ upstream of the polyA sequence.

In specific embodiments, the protein degradation peptide encoding sequence is positioned 5′ upstream of at least one (e.g., at least one, two, three, four, five, or more) of the two or more nuclease recognition sequences. For example, a recombinant DNA construct of the present disclosure may contain a protein degradation peptide encoding sequence that is positioned 5′ upstream of at least one of the two or more nuclease recognition sequences. In additional or alternative embodiments, the protein degradation peptide encoding sequence is positioned 3′ downstream of at least one of the two or more nuclease recognition sequences. For example, a recombinant DNA construct of the present disclosure may contain a protein degradation peptide encoding sequence that is positioned 3′ downstream of at least one of the two or more nuclease recognition sequences.

### 2.2.5. Examples of Recombinant DNA Constructs

The recombinant DNA construct and self-limiting recombinant virus of the present disclosure can contain one or more of the features described hereinabove. For example, a self-limiting recombinant virus of the present disclosure can be encoded by a recombinant DNA construct that contains:

(i) a first nuclease recognition sequence positioned 3′ downstream of a first promoter, wherein the first promoter is operably linked to a nucleic acid sequence encoding an engineered nuclease and drives the expression of the engineered nuclease in a target cell;

(ii) a nuclear localization signal positioned 3′ downstream of the first nuclease recognition sequence;

(iii) an intron positioned 3′ downstream of the nuclear localization signal and 5′ upstream of an exon encoding the nucleic acid sequence;

(iv) a second nuclease recognition sequence positioned 3′ downstream of the exon that encodes the nucleic acid sequence; and

(v) a polyA sequence positioned 3′ downstream of the second nuclease recognition sequence.

Alternatively, a self-limiting recombinant virus of the present disclosure can be encoded by a recombinant DNA construct that contains:

(i) a first nuclease recognition sequence positioned 3′ downstream of a first promoter, wherein the first promoter is operably linked to a nucleic acid sequence encoding an engineered nuclease and drives the expression of the engineered nuclease in a target cell;

(ii) a nuclear localization signal positioned 3′ downstream of the first nuclease recognition sequence;

(iii) an intron positioned 3′ downstream of the nuclear localization signal and 5′ upstream of an exon encoding the nucleic acid sequence;

(iv) a protein degradation peptide encoding sequence positioned 3′ downstream of the exon that encodes the nucleic acid sequence;

(v) a second nuclease recognition sequence positioned 3′ downstream of the protein degradation peptide encoding sequence; and

(vi) a polyA sequence positioned 3′ downstream of the second nuclease recognition sequence.

Alternatively, a self-limiting recombinant virus of the present disclosure can be encoded by a recombinant DNA construct that contains:

(i) a nuclear localization signal positioned 3′ downstream of a first promoter, wherein the first promoter is operably linked to a nucleic acid sequence encoding an engineered nuclease and drives the expression of the engineered nuclease in a target cell;

(ii) an intron positioned 3′ downstream of the nuclear localization signal and 5′ upstream of an exon encoding the nucleic acid sequence;

(iii) a first nuclease recognition sequence positioned within the intron;

(iv) a protein degradation peptide encoding sequence positioned 3′ downstream of the exon that encodes the nucleic acid sequence;

(v) a second nuclease recognition sequence positioned 3′ downstream of the protein degradation peptide encoding sequence; and

(vi) a polyA sequence positioned 3′ downstream of the second nuclease recognition sequence.

Alternatively, a self-limiting recombinant virus of the present disclosure can be encoded by a recombinant DNA construct that contains:

(i) a first nuclease recognition sequence positioned 3′ downstream of a first promoter, wherein the first promoter is operably linked to a nucleic acid sequence encoding an engineered nuclease and drives the expression of the engineered nuclease in a target cell;

(ii) a nuclear localization signal positioned 3′ downstream of the first nuclease recognition sequence;

(iii) an intron positioned 3′ downstream of the nuclear localization signal and 5′ upstream of an exon encoding the nucleic acid sequence;

(iv) a second nuclease recognition sequence positioned within the intron;

(v) a third nuclease recognition sequence positioned 3′ downstream of the exon that encodes the nucleic acid sequence; and

(vi) a polyA sequence positioned 3′ downstream of the third nuclease recognition sequence.

Alternatively, a self-limiting recombinant virus of the present disclosure can be encoded by a recombinant DNA construct that contains:

(i) a first nuclease recognition sequence positioned 3′ downstream of a first promoter, wherein the first promoter is operably linked to a nucleic acid sequence encoding an engineered nuclease and drives the expression of the engineered nuclease in a target cell;

(ii) a nuclear localization signal positioned 3′ downstream of the first nuclease recognition sequence;

(iii) an intron positioned 3′ downstream of the nuclear localization signal and 5′ upstream of an exon encoding the nucleic acid sequence;

(iv) a second nuclease recognition sequence positioned within the intron;

(v) a protein degradation peptide encoding sequence positioned 3′ downstream of the exon that encodes the nucleic acid sequence;

(vi) a third nuclease recognition sequence positioned 3′ downstream of the protein degradation peptide encoding sequence; and

(vii) a polyA sequence positioned 3′ downstream of the third nuclease recognition sequence.

### 2.3. Target Site Cleavage

Also disclosed herein is the use of a recombinant virus containing a recombinant DNA construct, such as a self-limiting recombinant virus of the present disclosure in cleaving a target site in the genome of a target cell. In some embodiments, a target site in the genome of a target cell can be cleaved by introducing a recombinant virus, such as a self-limiting recombinant virus of the present disclosure into the target cell. Introduction of a self-limiting recombinant virus of the present disclosure into the target cell leads to expression of the engineered nuclease in the target cell. Once expressed, the engineered nuclease may cleave the two or more nuclease recognition sequences on the recombinant DNA construct and/or in the genome of the target cell.

In some embodiments, cleavage of the two or more nuclease recognition sequences by the engineered nuclease on the recombinant DNA construct may increase on-target cleavage of the genome of the target cell. In certain embodiments, on-target cleavage of the genome of the target cell may be increased following the introduction of a self-limiting recombinant virus of the present disclosure, when compared to on-target cleavage of the genome of the target cell following introduction of a control recombinant virus that does not contain the two or more nuclease recognition sequences cleaved by the engineered nuclease. In particular embodiments, on-target cleavage of the genome of the target cell may be increased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, on-target cleavage of the genome of the target cell may be increased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, on-target cleavage of the genome of the target cell may be increased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences on the recombinant DNA construct of the self-limiting recombinant virus by the engineered nuclease in a target cell may decrease off-target cleavage by the engineered nuclease of the genome of the target cell. In certain embodiments, off-target cleavage of the genome of the target cell may be decreased following the introduction of a self-limiting recombinant virus of the present disclosure, when compared to off-target cleavage of the genome of the target cell following introduction of a control recombinant virus that does not contain the two or more nuclease recognition sequences cleaved by the engineered nuclease. In particular embodiments, off-target cleavage of the genome of the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, off-target cleavage of the genome of the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, off-target cleavage of the genome of the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences by the engineered nuclease in a target cell may cause a self-limiting recombinant virus of the present disclosure to have a lower persistence time in the target cell, when compared to the persistence time of a control recombinant virus which does not contain the two or more nuclease recognition sequences cleaved by the engineered nuclease. Persistence time can be calculated as the time following introduction of the self-limiting recombinant virus or DNA construct into the target cell. In certain embodiments, the persistence time of a self-limiting recombinant virus in the target cell is less than 20 weeks such as less than 20 weeks, 19 weeks, 18 weeks, 17 weeks, 16 weeks, 15 weeks, 14 weeks, 13 weeks, 12 weeks, 11 weeks, 10 weeks, 9 weeks, 8 weeks, 7 weeks, 6 weeks, 5 weeks, 4 weeks, 3 weeks, 2 weeks, or 1 week. Additionally, or alternatively, the persistence time of a self-limiting recombinant virus in the target cell can be about 10 weeks, 9 weeks, 8 weeks, 7 weeks, 6 weeks, 5 weeks, 4 weeks, 3 weeks, 2 weeks, 1 week, or less. In particular, the persistence time of a self-limiting recombinant virus of the present disclosure in the target cell can be about 2 weeks.

In some instances, an engineered nuclease may bind and cleave a genomic recognition sequence in a target cell. In some such embodiments, following cleavage of the two or more nuclease recognition sequences, integration of a self-limiting recombinant virus of the present disclosure into the genome of the target cell is reduced. In certain embodiments, integration of the self-limiting recombinant virus into the genome of the target cell is reduced, when compared to integration of a control recombinant virus that does not contain the two or more nuclease recognition sequences cleaved by the engineered nuclease. In particular embodiments, integration of the self-limiting recombinant virus into the genome of the target cell may be reduced following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, integration of the self-limiting recombinant virus into the genome of the target cell may be reduced by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, integration of the self-limiting recombinant virus into the genome of the target cell may be reduced by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences by the engineered nuclease in a target cell may decrease mRNA expression of the engineered nuclease in the target cell. In certain embodiments, mRNA expression of the engineered nuclease in the target cell may be decreased following the introduction of a self-limiting recombinant virus of the present disclosure, when compared to mRNA expression of the engineered nuclease in the target cell following introduction of a control recombinant virus that does not contain the two or more nuclease recognition sequences cleaved by the engineered nuclease. In particular embodiments, mRNA expression of the engineered nuclease in the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, mRNA expression of the engineered nuclease in the genome of the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, mRNA expression of the engineered nuclease in the genome of the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell. mRNA expression can be measured by measuring the level of mRNA of the engineered nuclease in the target cell by any means known in the art. For example, the level of mRNA of the engineered nuclease can be measured using quantitative RT-PCR.

In some embodiments, cleavage of the two or more nuclease recognition sequences by the engineered nuclease in a target cell may decrease protein expression of the engineered nuclease in the target cell. In certain embodiments, protein expression of the engineered nuclease in the target cell may be decreased following the introduction of a self-limiting recombinant virus of the present disclosure, when compared to protein expression of the engineered nuclease in the target cell following introduction of a control recombinant virus that does not contain the two or more nuclease recognition sequences cleaved by the engineered nuclease. In particular embodiments, protein expression of the engineered nuclease in the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, protein expression of the engineered nuclease in the genome of the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, protein expression of the engineered nuclease in the genome of the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences by the engineered nuclease in a target cell may decrease the copy number of recombinant virus in a target cell. In certain embodiments, copy number of recombinant virus in the target cell may be decreased following the introduction of the self-limiting recombinant virus of the present disclosure, when compared to copy number of recombinant virus in the target cell following introduction of a control recombinant virus that does not contain the two or more nuclease recognition sequences cleaved by the engineered nuclease. In particular embodiments, copy number of recombinant virus in the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, copy number of recombinant virus in the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, copy number of recombinant virus in the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences by the engineered nuclease in a target cell may decrease immunogenic effect of recombinant virus in a target cell. In certain embodiments, immunogenic effect of recombinant virus in the target cell may be decreased following the introduction of the self-limiting recombinant virus of the present disclosure, when compared to immunogenic effect of recombinant virus in the target cell following introduction of a control recombinant virus that does not contain the two or more nuclease recognition sequences cleaved by the engineered nuclease. In particular embodiments, immunogenic effect of recombinant virus in the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, immunogenic effect of recombinant virus in the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, immunogenic effect of recombinant virus in the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell.

In some embodiments, cleavage of the two or more nuclease recognition sequences by the engineered nuclease in a target cell may decrease genotoxic effect of recombinant virus in a target cell. In certain embodiments, genotoxic effect of recombinant virus in the target cell may be decreased following the introduction of the self-limiting recombinant virus of the present disclosure, when compared to genotoxic effect of recombinant virus in the target cell following introduction of a control recombinant virus that does not contain the two or more nuclease recognition sequences cleaved by the engineered nuclease. In particular embodiments, genotoxic effect of recombinant virus in the target cell may be decreased following at least 1 week (e.g., at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) after introduction of the self-limiting recombinant virus into the target cell. In specific embodiments, genotoxic effect of recombinant virus in the target cell may be decreased by at least 5% (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more) following the introduction of the self-limiting recombinant virus into the target cell. Additionally, or alternatively, genotoxic effect of recombinant virus in the target cell may be decreased by about 5-95% (e.g., about 10-90%, 15-85%, 20-80%, 25-75%, 30-70%, 35-65%, or 40-60%) or more, such as about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, following the introduction of the self-limiting recombinant virus into the target cell. In certain embodiments, genotoxic effect includes one or more of translocations, inversions, and/or insertions and deletions (indels).

The target cell can be any prokaryotic or eukaryotic cell. In some embodiments, the target cell is a eukaryotic cell, such as a mammalian cell or a plant cell. In certain embodiments, the eukaryotic cell is a mammalian cell, such as a human cell. In particular embodiments, the human cell is a T cell or a natural killer (NK) cell. In specific embodiments, the T cell is a primary T cell.

### 2.4. Producing Genetically-Modified Eukaryotic Cells

Also disclosed herein is the use of a recombinant virus and/or a recombinant DNA construct of the present disclosure in producing genetically-modified eukaryotic cells. The present disclosure provides methods for producing a genetically-modified eukaryotic cell by disrupting a target sequence in a genome of the eukaryotic cell. In some embodiments, the method involves introducing a recombinant DNA construct of the present disclosure into the eukaryotic cell, which results in expression of the engineered nuclease in the eukaryotic cell. Once expressed in the eukaryotic cell, the engineered nuclease produces a cleavage site in the genome at a target sequence comprising a genomic recognition sequence, and the target sequence is then disrupted by non-homologous end-joining at that cleavage site of the genomic recognition sequence. In certain embodiments, the recombinant DNA construct is introduced into the eukaryotic cell by a recombinant virus, such as a self-limiting recombinant virus described hereinabove.

Engineered nucleases of the invention can be delivered into a cell in the form of protein or, preferably, as a nucleic acid encoding the engineered nuclease. Such nucleic acids can be DNA (e.g., circular or linearized plasmid DNA or PCR products) or RNA (e.g., mRNA). Accordingly, polynucleotides are provided herein that comprise a nucleic acid sequence encoding an engineered nuclease disclosed herein. In some embodiments, a first engineered nuclease is encoded by a recombinant DNA construct described herein. In some embodiments, a second or additional engineered meganuclease is encoded by an mRNA polynucleotide. In specific embodiments, the polynucleotide is an mRNA. The polynucleotides encoding an engineered meganuclease disclosed herein can be operably linked to a promoter. In specific embodiments, expression cassettes are provided that comprise a promoter operably linked to a polynucleotide having a nucleic acid sequence encoding an engineered meganuclease disclosed herein.

For embodiments in which the engineered nuclease coding sequence is delivered in DNA form (e.g., as part of a recombinant DNA construct described herein), it should be operably linked to a promoter to facilitate transcription of the nuclease gene. Mammalian promoters suitable for the invention include constitutive promoters such as the cytomegalovirus early (CMV) promoter (Thomsen et al. (1984), Proc Natl Acad Sci USA. 81(3):659-63), the SV40 early promoter (Benoist and Chambon (1981), Nature. 290(5804):304-10), a CAG promoter, an EF1 alpha promoter, or a UbC promoter, as well as inducible promoters such as the tetracycline-inducible promoter (Dingermann et al. (1992), Mol Cell Biol. 12(9):4038-45). An engineered nuclease of the invention can also be operably linked to a synthetic promoter. Synthetic promoters can include, without limitation, the JeT promoter (WO 2002/012514). In specific embodiments, a nucleic acid sequence encoding an engineered nuclease of the invention is operably linked to a tissue-specific promoter, such as a muscle cell-specific promoter, a skeletal muscle-specific promoter, a myotube-specific promoter, a muscle satellite cell-specific promoter, a neuron-specific promoter, an astrocyte-specific promoter, a microglia-specific promoter, an eye cell-specific promoter, a retinal cell-specific promoter, a retinal ganglion cell-specific promoter, a retinal pigmentary epithelium-specific promoter, a pancreatic cell-specific promoter, or a pancreatic beta cell-specific promoter.

In some embodiments, mRNA encoding an engineered nuclease (e.g., a second engineered nuclease) is delivered to a cell because this reduces the likelihood that the gene encoding the engineered nuclease will integrate into the genome of the cell.

Such mRNA encoding an engineered nuclease can be produced using methods known in the art such as in vitro transcription. In some embodiments, the mRNA is 5′ capped using 7-methyl-guanosine, anti-reverse cap analogs (ARCA) (U.S. Pat. No. 7,074,596), CLEANCAP® analogs such as Cap 1 analogs (Trilink, San Diego, Calif.), or enzymatically capped using vaccinia capping enzyme or similar. In some embodiments, the mRNA may be polyadenylated. The mRNA may contain various 5′ and 3′ untranslated sequence elements to enhance expression the encoded engineered nuclease and/or stability of the mRNA itself. Such elements can include, for example, posttranslational regulatory elements such as a woodchuck hepatitis virus posttranslational regulatory element. The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

Purified nuclease proteins can be delivered into cells to cleave DNA by a variety of different mechanisms known in the art, including those further detailed herein.

In another particular embodiment, a nucleic acid encoding a nuclease of the invention is introduced into the cell using a single-stranded DNA template. The single-stranded DNA can further comprise a 5′ and/or a 3′ AAV inverted terminal repeat (ITR) upstream and/or downstream of the sequence encoding the engineered nuclease. The single-stranded DNA can further comprise a 5′ and/or a 3′ homology arm upstream and/or downstream of the sequence encoding the engineered nuclease.

In another particular embodiment, genes encoding a nuclease of the invention is introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below.

Additionally, the present disclosure provides methods for producing a genetically-modified eukaryotic cell by disrupting a target sequence in a genome of the eukaryotic cell. In some embodiments, the method involves introducing an engineered nuclease into the eukaryotic cell, wherein the engineered nuclease is encoded by a recombinant DNA construct of the present disclosure. Once introduced in the eukaryotic cell, the engineered nuclease produces a cleavage site in the genome at a genomic recognition sequence of a genomic target sequence, and the target sequence is then disrupted by non-homologous end-joining at that cleavage site of the genomic recognition sequence.

Additionally, the present disclosure provides methods for producing a genetically-modified eukaryotic cell, such as a eukaryotic cell, that contains an exogenous sequence of interest inserted into its genome. In some embodiments, the method involves introducing in the eukaryotic cell the following: (i) a recombinant DNA construct of the present disclosure that encodes an engineered nuclease; and (ii) a second recombinant DNA construct that encodes the sequence of interest. Following introduction of the recombinant DNA constructs in the eukaryotic cell, the engineered nuclease is expressed in the eukaryotic cell and produces a cleavage site in the genome at a genomic recognition sequence of a target site. The sequence of interest encoded by the second recombinant DNA construct is then inserted into the genome at that cleavage site. In certain embodiments, the second recombinant DNA construct may further contain sequences homologous to sequences flanking the cleavage site. In some such embodiments, the sequence of interest may then be inserted at the cleavage site by homologous recombination. In some instances, the recombinant DNA construct and/or the second recombinant DNA construct may be introduced in the target cell by a recombinant virus, such as a self-limiting recombinant virus described hereinabove.

Additionally, the present disclosure provides methods for producing a genetically-modified eukaryotic cell, such as a eukaryotic cell that contains an exogenous sequence of interest inserted into its genome. In some embodiments, the method involves introducing in the eukaryotic cell the following: (i) an engineered nuclease, such as an engineered nuclease encoded by a recombinant DNA construct of the present disclosure; and (ii) a nucleic acid sequence that includes the sequence of interest. Once introduced in the eukaryotic cell, the engineered nuclease produces a cleavage site in the genome at a genomic recognition sequence, and the sequence of interest is then inserted into the genome at that cleavage site. In certain embodiments, the nucleic acid sequence of “(ii)” may further contain sequences homologous to sequences flanking the cleavage site. In some such embodiments, the sequence of interest may then be inserted at that cleavage site by homologous recombination. In certain embodiments, the nucleic acid sequence of “(ii)” may be introduced into the eukaryotic cell by a recombinant virus, such as a self-limiting recombinant virus described hereinabove.

The recombinant DNA constructs encoding engineered nucleases, can be delivered into cells to cleave genomic DNA by a variety of different mechanisms known in the art, including those further detailed herein below.

In some embodiments, the recombinant DNA constructs encoding nuclease proteins are formulated for systemic administration, or administration to target tissues, in a pharmaceutically acceptable carrier in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, the DNA construct is typically admixed with a pharmaceutically acceptable carrier. The carrier must, of course, be acceptable in the sense of being compatible with any other ingredients in the formulation and must not be deleterious to the patient. The carrier can be a solid or a liquid, or both, and can be formulated with the compound as a unit-dose formulation.

In some embodiments, the recombinant DNA constructs encoding nuclease proteins are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, the recombinant DNA constructs encoding nuclease proteins, are coupled covalently or non-covalently to an antibody that recognizes a specific cell-surface receptor expressed on target cells such that the recombinant DNA constructs encoding nuclease proteins bind to and are internalized by the target cells. Alternatively, the recombinant DNA constructs encoding nuclease proteins can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, the recombinant DNA constructs encoding nuclease proteins are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, the recombinant DNA constructs encoding nuclease proteins are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the recombinant DNA constructs encoding nuclease proteins can be attached to or encapsulated with the nanoparticle core. This increases the copy number of the DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the recombinant DNA constructs encoding nuclease proteins are encapsulated within liposomes or complexed using cationic lipids (see, e.g., LIPOFECTAMINE™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, the recombinant DNA constructs encoding nuclease proteins are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, the recombinant DNA constructs encoding nuclease proteins are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, the recombinant DNA constructs encoding nuclease proteins are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, the recombinant DNA constructs encoding nuclease proteins are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

Also provided herein is a genetically-modified eukaryotic cell that is produced by one or more methods disclosed hereinabove. In some embodiments, a eukaryotic cell described hereinabove is a plant cell. In other embodiments, a eukaryotic cell described hereinabove is a mammalian cell, such as a human cell. In particular embodiments, the human cell is a T cell or a natural killer (NK) cell. In specific embodiments, the T cell is a primary T cell.

### 2.5 Pharmaceutical Compositions

In some embodiments, the invention provides a pharmaceutical composition comprising a pharmaceutically acceptable carrier and a recombinant DNA construct comprising a nucleic acid sequence encoding an engineered nuclease of the invention. In some embodiments, the invention provides a pharmaceutical composition comprising a pharmaceutically acceptable carrier and a recombinant virus comprising a recombinant DNA construct described herein comprising a nucleic acid sequence encoding an engineered nuclease described herein. In particular, pharmaceutical compositions are provided that comprise a pharmaceutically acceptable carrier and a therapeutically effective amount of a nucleic acid encoding an engineered meganuclease or an engineered meganuclease peptide.

In other embodiments, the invention provides a pharmaceutical composition comprising a pharmaceutically acceptable carrier and a genetically-modified cell of the invention. The genetically modified cell can be delivered to a desired target tissue where the cell.

Pharmaceutical compositions of the invention can be useful for treating a subject having a disease in a subject in need of treatment thereof in accordance with the present invention.

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, nuclease polypeptides (or DNA/RNA encoding the same or cells expressing the same) are typically admixed with a pharmaceutically acceptable carrier, and the resulting composition is administered to a subject. The carrier must be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

In particular embodiments of the invention, the pharmaceutical composition comprises a recombinant virus comprising a polynucleotide (e.g., a viral genome) comprising a nucleic acid sequence encoding an engineered nuclease described herein. Such recombinant viruses are known in the art and include recombinant retroviruses, recombinant lentiviruses, recombinant adenoviruses, and recombinant adeno-associated viruses (AAV) (reviewed in Vannucci, et al. (2013 New Microbiol. 36:1-22). Recombinant AAVs useful in the invention can have any capsid or serotype that allows for transduction of the virus into a target cell type and expression of the meganuclease gene by the target cell. For example, in some embodiments, recombinant AAV has a serotype of AAV1, AAV2, AAV3, AAV3B, AAV4, AAV5, AAV6, AAV7, AAV8, AAV9, AAV10, AAV11, AAV RH74m or AAVHSC. In some embodiments, the recombinant virus is injected directly into target tissues. In alternative embodiments, the recombinant virus is delivered systemically via the circulatory system. It is known in the art that different AAVs tend to localize to different tissues, and one could select an appropriate AAV capsid/serotype for preferential delivery to a particular tissue. Accordingly, in some embodiments, the AAV serotype is AAV1. Accordingly, in some embodiments, the AAV serotype is AAV2. In some embodiments, the AAV serotype is AAV3. In some embodiments, the AAV serotype is AAV3B. In some embodiments, the AAV serotype is AAV4. In some embodiments, the AAV serotype is AAV5. In some embodiments, the AAV serotype is AAV6. In some embodiments, the AAV serotype is AAV7. In some embodiments, the AAV serotype is AAV8. In some embodiments, the AAV serotype is AAV9. In some embodiments, the AAV serotype is AAV10. In some embodiments, the AAV serotype is AAV11. In some embodiments, the AAV serotype is AAV RH74. In some embodiments, the AAV serotype is AAVHSC. AAVs can also be self-complementary such that they do not require second-strand DNA synthesis in the host cell (McCarty, et al. (2001) Gene Ther. 8:1248-54). Nucleic acids delivered by recombinant AAVs can include left (5′) and right (3′) inverted terminal repeats.

In particular embodiments of the invention, the pharmaceutical composition comprises one or more mRNAs described herein (e.g., mRNAs encoding engineered nucleases) formulated within lipid nanoparticles.

The selection of cationic lipids, non-cationic lipids and/or lipid conjugates which comprise the lipid nanoparticle, as well as the relative molar ratio of such lipids to each other, is based upon the characteristics of the selected lipid(s), the nature of the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S. Patent Publication Nos. 20040142025 and 20070042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm or from about 60 nm to about 120 nm. The size of the lipid nanoparticles may be determined by quasi-electric light scattering (QELS) as described in Bloomfield, Ann. Rev. Biophys. Bioeng., 10:421\hat{ }150 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-di stearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid comprises from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) comprises from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-dilauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-w-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol (or any fraction thereof or range therein) of the total lipid present in the particle. Typically, in such instances, the PEG moiety has an average molecular weight of about 750 Daltons.

In other embodiments, the composition comprises amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Choi 3-β-[N—(N′,N′-dimethylmethane) carbamoyl]cholesterol, TC-Choi 3-β-[N—(N′,N′,N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DOME 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemi succinate), Mo-Chol (morpholine-N-ethylamino-cholesterol hemi succinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

### 2.6. Kinetic Balancing

In some cases, it may be advantageous to modify the recognition sequence in the self-limiting recombinant virus to make it sub-optimal. The recombinant virus should not be cut before a sufficient concentration of the engineered nuclease accumulates in the cell, such as in a target cell, to modify the cell's genome in the desired manner. Because the chromosomal target sequence of interest (i.e., comprising the genomic recognition sequence) will be chromatinized, it is more difficult to access than an episomal vector sequence. Thus, higher concentrations of the engineered nuclease are likely required to cut the genomic recognition sequence, such as a chromosomal recognition site in the genome of the target cell. If the transcribed engineered nuclease binds and cleaves the recognition sequence in the self-limiting recombinant virus before the appropriate amount of engineered nuclease is achieved, binding and cleavage of the genomic recognition sequence within the cell may be unrealized. The use of sub-optimal recognition sequences in the recombinant virus is referred to as “kinetic balancing,” because it helps coordinate the timing of DNA cleavage such that the genome of the target cell is cut first, followed by the genome of the virus.

In general, sub-optimal recognition sequences can be generated by deviating from the precise sequence that the engineered nuclease was engineered to recognize while still maintaining cleavage specificity of the genomic recognition sequence. An engineered nuclease, such as an engineered meganuclease, for example, recognizes a 22 bp sequence but will tolerate certain 1-2 basepair changes in its preferred sequence. These modified sequences are typically cut less efficiently than the preferred sequence and, so, are suitable for incorporation into self-limiting recombinant virus. In selecting a sub-optimal recognition sequence for incorporation into self-limiting recombinant virus, it is critical that the sub-optimal site is still cut by the engineered nuclease, albeit less efficiently than the preferred sequence. For each of the engineered nuclease types, regions of a recognition sequence may be able to tolerate changes. For example, engineered meganucleases tolerate single-base changes at bases 1, 10, 11, 12, 13, and 22 of the recognition sequence (Jurica et al., Mol Cell 2:469-76 (1998)).

Experimental methods to evaluate and quantify site-specific DNA cleavage may be performed, including in vitro DNA digests with purified nuclease protein and cell-based reporter assays (Chevalier et al., J Mol Biol 329: 253-69 (2003)). These methods can be used to evaluate a variety of sub-optimal recognition sequences to determine the sequences that are cut less efficiently than the preferred recognition sequence in the genome of the cell.

Alternative methods for achieving kinetic balancing include changing a center sequence, which is more readily cleaved by an engineered meganuclease for a center sequence that is less readily cleaved by the engineered meganuclease. In this way, the two −9 recognition half-site sequences are kept constant and only the four base pair center sequence is changed. Accordingly, it may only be necessary to express one engineered nuclease which has an alternative cleavage rate towards a genomic recognition sequence and one or more construct recognition sequences. In some embodiments, a genomic recognition sequence comprises a four base pair center sequence, which is cleaved by an engineered meganuclease at a higher rate than one or more of the construct recognition sequences. Alternatively, in some embodiments, a construct recognition sequence comprises a four base pair center sequence, which is cleaved by an engineered meganuclease at a higher rate than a genomic recognition sequence.

### 2.6. Methods for Producing Self-Limiting Viruses

rAAV virus is typically produced in mammalian cell lines such as HEK-293. Because the viral cap and rep genes are removed from the vector to prevent its self-replication to make room for the therapeutic gene(s) to be delivered (e.g., the engineered nuclease gene), it is necessary to provide these in trans in the packaging cell line. In addition, it is necessary to provide the “helper” (e.g., adenoviral) components necessary to support replication (Cots et al., Curr Gene Ther 13: 370-81 (2013)). Frequently, rAAV is produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because rAAV particles are typically produced (manufactured) in cells, precautions must be taken in practicing the current invention to ensure that the site-specific engineered nuclease is not expressed in the packaging cells. Because the viral genomes of the present disclosure comprise a recognition sequence for the engineered nuclease, any engineered nuclease expressed in the packaging cell line will be capable of cleaving the viral genome before it can be packaged into viral particles. This will result in reduced packaging efficiency and/or the packaging of fragmented genomes. Several approaches can be used to prevent expression of the engineered nuclease in the packaging cells. The engineered nuclease can be placed under the control of any promoter that is capable of expressing the gene encoding the nuclease. In some embodiments, the promoter is a constitutive promoter, or the promoter is a tissue-specific promoter such as, for example, a stem cell-specific promoter, a CD34+ HSC-specific promoter, a muscle-specific promoter, a skeletal muscle-specific promoter, a myotube-specific promoter, a muscle satellite cell-specific promoter, a neuron-specific promoter, an astrocyte-specific promoter, a microglia-specific promoter, an eye cell-specific promoter, a retinal cell-specific promoter, a retinal ganglion cell-specific promoter, a retinal pigmentary epithelium-specific promoter, a pancreatic cell-specific promoter, a pancreatic beta cell-specific promoter, a kidney cell-specific promoter, a bone marrow cell-specific promoter, or an ear hair cell-specific promoter. In some embodiments, the constitutive promoter is a CMV promoter, a CAG promoter, an EF1 alpha promoter, or a UbC promoter.

In some embodiments, the engineered nuclease can be placed under the control of a tissue-specific promoter that is not active in the packaging cells. For example, if a self-limiting recombinant virus is developed for delivery of (an) engineered nuclease gene(s) to muscle tissue, a muscle-specific promoter can be used. Examples of muscle-specific promoters include C5-12 (Liu et al., Hum Gene Ther 15:783-92 (2004)), the muscle-specific creatine kinase (MCK) promoter (Yuasa et al., Gene Ther. 9:1576-88 (2002)), or the smooth muscle 22 (SM22) promoter (Haase et al., BMC Biotechnol. 13:49-54 (2013)). Examples of CNS (neuron)-specific promoters include the NSE, Synapsin, and MeCP2 promoters (Lentz et al., Neurobiol Dis. 48:179-88 (2012)). Examples of liver-specific promoters include albumin promoters (such as Palb), human α1-antitrypsin (such as Pa1AT), and hemopexin (such as Phpx) (Kramer et al., Mol Therapy 7:375-85 (2003)). In particular, liver-specific promoters for use in the compositions and methods described herein may be one or more of a human thyroxine binding globulin (TBG) promoter, a human alpha-1 antitrypsin promoter, a hybrid liver specific promoter, or an apolipoprotein A-II promoter. Examples of eye-specific promoters include opsin, and corneal epithelium-specific K12 promoters (Martin et al., Methods (28): 267-75 (2002); Tong et al., J Gene Med, 9:956-66 (2007)). These promoters, or other tissue-specific promoters known in the art, are not highly-active in HEK-293 cells and, thus, will not expected to yield significant levels of endonuclease gene expression in packaging cells when incorporated into self-limiting recombinant virus of the present disclosure. Similarly, the self-limiting recombinant virus of the present invention contemplate the use of other cell lines with the use of incompatible tissue specific promoters (i.e., the HeLa cell line (human epithelial cell) and using the liver-specific hemopexin promoter). Other examples of tissue specific promoters include: synovial sarcomas PDZD4 (cerebellum), C6 (liver), ASB5 (muscle), PPP1R12B (heart), SLC5A12 (kidney), cholesterol regulation APOM (liver), ADPRHL1 (heart), and monogenic malformation syndromes TP73L (muscle) (Jacox et al., PLoS One v.5(8):e12274 (2010)).

Alternatively, the recombinant virus can be packaged in cells from a different species in which the engineered nuclease is not likely to be expressed. For example, viral particles can be produced in microbial, insect, or plant cells using mammalian promoters, such as the cytomegalovirus- or SV40 virus-early promoters, which are not active in the non-mammalian packaging cells. In a particular embodiment, viral particles are produced in insect cells using the baculovirus system as described by Gao, et al. (Gao et al., J. Biotechnol. 131(2):138-43 (2007)). An engineered nuclease under the control of a mammalian promoter is unlikely to be expressed in these cells (Airenne et al., Mol. Ther. 21(4):739-49 (2013)). Moreover, insect cells utilize different mRNA splicing motifs than mammalian cells. Thus, it is possible to incorporate a mammalian intron, such as the human growth hormone (HGH) intron, or the SV40 large T antigen intron, into the coding sequence of an engineered nuclease (see, for example, FIG. 1). Because these introns are not spliced efficiently from pre-mRNA transcripts in insect cells, insect cells will not express a functional engineered nuclease and will package the full-length genome. In contrast, mammalian cells to which the resulting rAAV particles are delivered will properly splice the pre-mRNA and will express functional engineered nuclease protein. Haifeng Chen has reported the use of the HGH and SV40 large T antigen introns to attenuate expression of the toxic proteins barnase and diphtheria toxin fragment A in insect packaging cells, enabling the production of rAAV carrying these toxin genes (Chen, Mol Ther Nucleic Acids 1(11): e57

Additionally, or alternatively, an engineered nuclease gene can be operably linked to an inducible promoter, such that a small-molecule inducer is required for expression of the engineered nuclease. Examples of inducible promoters include the Tet-On system (Clontech; Chen et al., BMC Biotechnol. 15(1):4 (2015)) and the RheoSwitch system (Intrexon; Sowa et al., Spine, 36(10): E623-8 (2011)). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the engineered nuclease gene under the control of a promoter that responds to the corresponding transcription factor, the engineered nuclease gene having (a) binding site(s) for the transcription factor; and 2) including the gene encoding the transcription factor in the packaged viral genome. The latter step is necessary because the engineered nuclease will not be expressed in the target cells or tissues following rAAV delivery if the transcription activator is not also provided to the same cells. The transcription activator then induces engineered nuclease gene expression only in cells or tissues that are treated with the cognate small-molecule activator. This approach is advantageous because it enables expression of the engineered nuclease gene to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, rAAV particles are produced in a mammalian cell line that expresses a transcription repressor that prevents expression of the engineered nuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current disclosure, packaging cells are transfected/transduced with a recombinant virus encoding a transcription repressor and the engineered nuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or, most preferably, it can be stably integrated into the genome of the packaging cell such that it is expressed constitutively. Some methods to modify common mammalian promoters to incorporate transcription repressor sites have been disclosed in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson, Gene 183:137-42 (1996)). The use of a non-human transcription repressor ensures that transcription of the endonuclease gene will be repressed only in the packaging cells expressing the repressor and not in target cells or tissues transduced with the resulting self-limiting rAAV.

### 2.7. Methods for Delivering Self-Limiting Recombinant Virus to Human Patients and Animals

The self-limiting recombinant virus of the invention, with their significant safety advantages relative to conventional gene-therapy vectors, can be used as therapeutic agents for the treatment of genetic disorders. For therapeutic applications, route of administration is an important consideration. These self-limiting recombinant virus particles may be delivered systemically via intravenous injection, especially where the target tissues for the therapeutic are liver (e.g., hepatocytes) or vascular epithelium/endothelium. Alternatively, the self-limiting recombinant virus of the invention may be injected directly into target tissues. For example, rAAV can be delivered to muscle cells via intramuscular injection (Maltzahn, et al. (2012) Proc Natl Acad Sci USA. 109:20614-9), or hydrodynamic injection (Taniyama, et al. (2012) Curr Top Med Chem. 12:1630-7 and Hegge, et al. (2010) Hum Gene Ther. 21:829-42). Delivery to CNS can be accomplished by systemic delivery or intracranial injection (Weinberg, et al. (2013) Neuropharmacology. 69:82-8, Bourdenx, et al. (2014) Front Mol Neurosci 0.7:50, and Ojala D S, et al. (2015) Neuroscientist. 21(1):84-98). Direct injection (e.g. subretinal injection) is the preferred route of administration for the eye (Willett K and Bennett J (2013) Front Immunol. 4:261 and Colella P and Auricchio A (2012) Hum Gene Ther. 23(8):796-807.). Thus, as described herein the self-limiting viral particles may be administered by an administration route comprising intravenous, intramuscular, intraperitoneal, subcutaneous, intrahepatic, transmucosal, transdermal, intraarterial, and sublingual.

In some embodiments, a therapeutically effective amount of an engineered nuclease described herein is administered to a subject in need thereof. As appropriate, the dosage or dosing frequency of the engineered nuclease may be adjusted over the course of the treatment, based on the judgment of the administering physician. Appropriate doses will depend, among other factors, on the specifics of any AAV vector chosen (e.g., serotype, etc.), on the route of administration, on the subject being treated (i.e., age, weight, sex, and general condition of the subject), and the mode of administration. Thus, the appropriate dosage may vary from patient to patient. An appropriate effective amount can be readily determined by one of skill in the art. Dosage treatment may be a single dose schedule or a multiple dose schedule. Moreover, the subject may be administered as many doses as appropriate. One of skill in the art can readily determine an appropriate number of doses. The dosage may need to be adjusted to take into consideration an alternative route of administration or balance the therapeutic benefit against any side effects.

### 2.8. Self-Limiting Recombinant Adenovirus and Retrovirus

While particular embodiments of the invention are self-limiting rAAV, the same principles can be applied to recombinant adenovirus and recombinant lentivirus/retrovirus to limit the persistence times of these recombinant virus particles in cells. These recombinant virus particles have significantly larger genomes and, hence, larger “carrying capacities” than AAV which makes them preferable for the delivery of larger gene payloads to the cell. Indeed, for applications involving the use of a gene editing engineered nuclease to insert a transgene into the genome, recombinant adenovirus or lentivirus/retrovirus are preferred when the transgene is larger than ˜3.5 kb. For other applications, recombinant adenovirus or lentivirus/retrovirus are preferred when the gene editing engineered nuclease is too large to be encoded by rAAV. This is particularly applicable when employing TALENs and most CRISPR/Cas9 nucleases.

Adenovirus and lentiviruses/retroviruses naturally integrate into the genome of the host cell. To be useful for the present disclosure, the ability of the virus to integrate into the genome must be attenuated. For recombinant adenovirus or lentivirus/retrovirus, this is accomplished by mutating the int gene encoding the virus integrase. For example, Bobis-Wozowicz et al., used an integration-deficient recombinant retrovirus to deliver zinc-finger nucleases to human and mouse cells (Bobis-Wozowicz et al., Nature Scientific Reports 4:4656 (2014); Qasim et al., Mol Ther 18:1263-67(2010); Wanisch and Yáñez-Muñoz, Mol Ther 17(8):1316-32 (2009); Nowrouzi et al., Viruses 3(5):429-55 (2011)).

### 2.9. Use of Self-Limiting Recombinant Virus

A self-limiting recombinant virus disclosed herein can be used to infect cells, tissues, or organisms to achieve a multitude of therapeutic results. For instance, a self-limiting recombinant virus can be used to disable (“knock-out”) a gene. In infected cells, the expressed engineered nuclease within the cell recognizes a target sequence within a coding sequence of a gene of interest within the cell (that cell being part of a cell line, tissue, or organism) and cuts the DNA. The DNA break in the cell's genome will then be repaired by non-homologous end-joining (NHEJ), such that mutations are introduced at the target site that disables the gene of interest's function. Subsequently, the expressed engineered nuclease recognizes and cuts the self-limiting recombinant virus at the nuclease recognition sequence. Once cut, the self-limiting recombinant virus cannot produce concatemers that may otherwise form and persist within the episomes. Accordingly, the self-limiting recombinant virus will cease to persist within the target cell.

In other embodiments, a self-limiting recombinant virus may comprise, starting at a 5′ position between the ITRs, a promoter, a first engineered nuclease encoding sequence (such as a nucleic acid sequence encoding a first engineered nuclease) and polyA, a nuclease recognitions sequence, a second promoter (which may be the same as the first), an accessory engineered nuclease encoding sequence (such as an accessory nucleic acid sequence encoding an accessory engineered nuclease) with polyA followed by the 3′ ITR. Notably, this configuration may be altered, specifically the location of the nuclease recognition sequence (as discussed above and exemplified in FIG. 1). The nuclease recognition sequence may be recognized by any one of the engineered nucleases coded for within the self-limiting recombinant virus. In some embodiments, the nuclease recognition sequence is recognized by the first engineered nuclease. Moreover, it is contemplated that more than two engineered nucleases, each recognizing a different nuclease recognition sequence (such as non-identical nuclease recognition sequence) within a cell genome, may be housed within the self-limiting recombinant virus. In some embodiments, a recombinant virus may code for two engineered nucleases, such as a first engineered nuclease and an accessory engineered nuclease. Upon expression of the engineered nucleases in an infected cell, each engineered nuclease may recognize their own recognition sequences; for example, the first engineered nuclease may recognize one nuclease recognition sequence, and the accessory engineered nuclease may recognize the second nuclease recognition sequence. The engineered nucleases may cut the genome at each nuclease recognition sequence, exposing the region of interest coded between each cut site. The region of interest fragment can then be excised and degraded by cell machinery, and the cell genome may be repaired by re-ligation. As stated above, the ligation of the genome may be achieved by cell processes that maintain the integrity of the sequence and does not introduce additional sequence to the genome. The engineered nuclease may subsequently recognize the nuclease recognition sequence within the self-limiting recombinant virus and cut the viral genome. Once cut, the self-limiting recombinant virus cannot produce concatemers that may otherwise form and persist within the episomes. Hence, the self-limiting recombinant virus will cease to persist within the cell.

The self-limiting recombinant virus of the present invention may be employed to introduce a new transgene into the infected cell's genome. In these embodiments, the self-limiting recombinant virus comprises from a 5′ position between the ITRs: a promoter, an engineered nuclease encoding sequence (such as a nucleic acid sequence encoding an engineered nuclease) and polyA, a nuclease recognition sequence, a homologous DNA sequence, the transgene, and another homologous sequence at the 3′ position within the ITRs. Notably, this configuration may be altered, specifically the location of the nuclease recognition sequence (as discussed above and exemplified in FIG. 1). For example, at least one nuclease recognition sequence could be positioned on the 3′ end of the homologous DNA sequence of the transgene, or in an intron contained within the transgene sequence. After infection, the engineered nuclease is expressed within the cell, and recognizes a genomic recognition sequence within a region of interest. The engineered nuclease cleaves the genomic recognition sequence within the gene of interest, exposing 5′ and 3′ ends of the cell genome. The self-limiting recombinant virus contains matching homologous DNA for the exposed 5′ and 3′ ends of the cleaved cell genome. By homologous recombination, the homologous DNA regions flanking the 5′ and 3′ ends of the transgene recombine with the cleaved portion of the cell genome and the transgene of the self-limiting recombinant virus is inserted within the cell genome. The engineered nuclease subsequently recognizes the nuclease recognition sequence within the self-limiting recombinant virus and cuts the viral genome. Once cut, the self-limiting recombinant virus cannot produce concatemers that may otherwise form and persist within the episomes. The self-limiting recombinant virus will cease to persist within the cell.

In some embodiments, a self-limiting recombinant virus of the present disclosure may be dispatched to alter a gene sequence within a cell genome (as previously defined, that cell being part of a cell line, tissue, or organism). In these embodiments, the self-limiting recombinant virus comprises at a 5′ position of the ITRs, a promoter, an engineered nuclease encoding sequence (such as a nucleic acid sequence encoding an engineered nuclease) with polyA, a nuclease recognition sequence, a gene sequence and the 3′ position within the ITRs. The expressed engineered nuclease recognizes and cuts a site within the gene sequence in the infected cell's genome. This cut exposes 5′ and 3′ ends of the infected cell's genome that can recombine with the 5′ and 3′ ends of the gene sequence encoded within the self-limiting recombinant virus. The gene sequence is inserted into the infected cell's genome through homologous recombination. The engineered nuclease subsequently recognizes the nuclease recognition sequence within the self-limiting viral vector and cuts the viral genome. Once cut, the self-limiting recombinant virus cannot produce concatemers that may otherwise form and persist within the episomes. Hence, the self-limiting recombinant virus will cease to persist within the target cell.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1. Insertion of Target Site (TS) and/or PEST in AAV Genome Reduces Nuclease Expression and AAV Copy Number

### Materials and Methods

Vector Cloning Design

The goal of this experiment and experiments described in the following examples was to determine if reduction of nuclease expression levels lead to reduction of nuclease cleavage at off-target sites while maintaining measurable nuclease activity in mouse liver. Two mechanisms were used to reduce nuclease expression. One mechanism was to fuse a gene fragment encoding PEST tag to the nuclease open reading frame (ORF) so that the nuclease protein contains a PEST tag upon expression. The function of PEST tag is to facilitate the nuclease degradation, thus reducing the apparent nuclease levels. This mechanism has no effect on AAV copy number and persistence. Another mechanism is to insert more than two nuclease recognition sequences, also referred to herein as nuclease target sites (TS), in the AAV genome whose positions are chosen to break the nuclease ORF so that it prevents functional nuclease expression under rare event of integration into host genome. Under this mechanism, the nuclease expressed from an AAV can cut its therapeutic TS in genome, and the TS inserted in AAV genome to reduce AAV copy numbers, which subsequently results in less nuclease expression. Therefore, TS insertion can lead to reduction of AAV copy number and persistence as well as reduction of nuclease expression. In this experiment, two or three copies of nuclease TS were inserted in the AAV genome. In addition, three constructs combined insertion of both TS and PEST tag to determine the effect of two mechanisms. In total, six constructs were generated with 2-3 copies of TS and/or PEST tag inserted and one additional construct (NoTS) without any insertion as positive control. A schematic diagram of the constructs is provided in FIG. 1.

In all constructs, the nuclease expression is under control of TBG promoter, a liver specific promoter, to restrict nuclease expression in other cells/tissues transduced by AAV. A nuclear localization sequence (NLS) is attached to nuclease ORF to facilitate nuclear transport of nuclease after expression. An intron is inserted in nuclease ORF to restrict nuclease expression in non-mammalian cells. A SV40 polyadenylation signal sequence (SV40 polyA) is attached after the nuclease ORF to terminate transcription and add polyadenylation tail to nuclease mRNA. The nuclease exemplified in these experiments is an engineered meganuclease referred to as HAO 1-2L.30 (SEQ ID NO: 38), which was disclosed in PCT/US2019/68186. This engineered meganuclease binds and cleaves the 22 base pair recognition sequence according to SEQ ID NO: 25. These genetic elements, including promoters (tissue specific or constitutive), NLS and polyA sequence, can be changed and modulated for maximum therapeutic benefits based on the needs of different applications. All constructs were packaged into AAV serotype 8 (AAV8) vectors, which is an AAV serotype for liver transduction. The viral titers were determined by quantitative PCR (qPCR) using a primer pair (forward 5′ GAGTTTGGACAAACCACAACTAGA 3′ (SEQ ID NO: 1) and reverse 5′ AGCAATAGCATCACAAATTTCACAA 3′ (SEQ ID NO: 2)). The qPCR reaction was set up using KAPA SYBR® FAST qPCR Kit (KAPA Biosystems, Cat #KK4602), and run on QuantStudio 7 Flex Real-Time PCR Systems from ThermoFisher.

Mouse Strain and AAV Administration

8-week old female FVB mice were obtained and maintained in Vivarium animal facility. Each AAV8 vector was diluted to 1E+12 vg/mL in PBS. 2E+11 viral genomes (vg) of AAV8 vector was injected into each mouse by intravenous administration through the tail vein. In total, seven groups of mice (6 mice per group) were treated with AAV and one group of mice was treated with PBS as negative control. Two mice from each group were sacrificed, and their liver samples were collected 2-, 6- and 10-week post AAV administration. All the liver samples were stored in −80° C. freezer before processing.

Protein Extraction from Mouse Liver and Western Blot

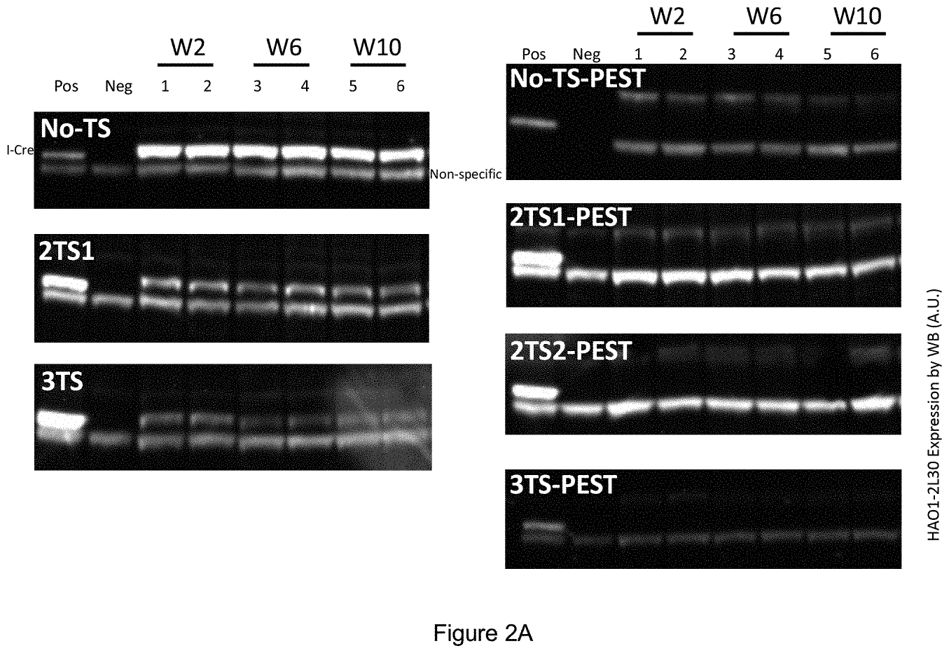

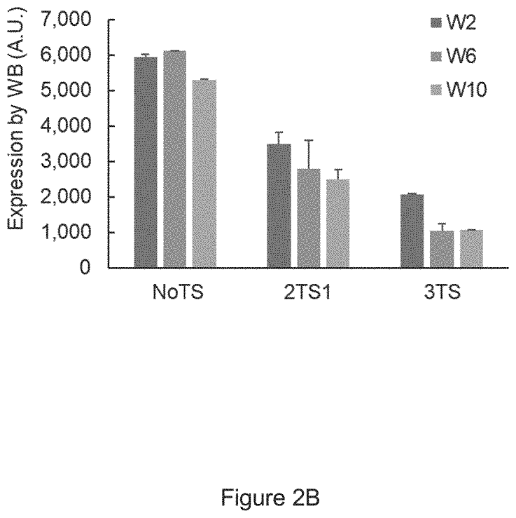

To extract total protein, small piece of mouse liver tissues were added to 1.5 mL tubes containing 300 μL RIPA buffer (EMD Millipore Cat #20-188) containing Protease Inhibitors (Roche Cat #11836153001), homogenized using a doweling rod, incubated on ice for 60 min, centrifuged 10 min at 14k×g, and the clarified lysates were transferred to a new 1.5 mL tube. Protein concentrations were determined by Pierce™ BCA Protein Assay Kit (ThermoFisher Cat #23227). 30 μg lysates were denatured under reducing conditions, resolved on a NuPAGE 10% Bis-Tris gel (ThermoFisher Cat #NP0301BOX), and transferred to a PVDF membrane (Invitrogen Cat #88520). The membranes were processed using the SuperSignal Western Blot Enhancer protocol (ThermoFisher Cat #46640). The primary antibody-rabbit anti I-Cre (Precision) was used at 1:8000 and secondary-goat anti rabbit HRP (Invitrogen Cat #G-21234) was used at 1:50k. The blots were incubated 5 min in ECL Prime western blot detection reagent (GE Healthcare Cat #RPN2232) and images were captured using a UVP ChemiDoc 815 Imager. Images from the western blot analysis are provided in FIG. 2A. Nuclease expression levels were quantified by measuring the band intensity using ImageJ software. Results from quantification of nuclease expression levels are provided in FIG. 2B.

mRNA Extraction from Mouse Liver and qRT-PCR

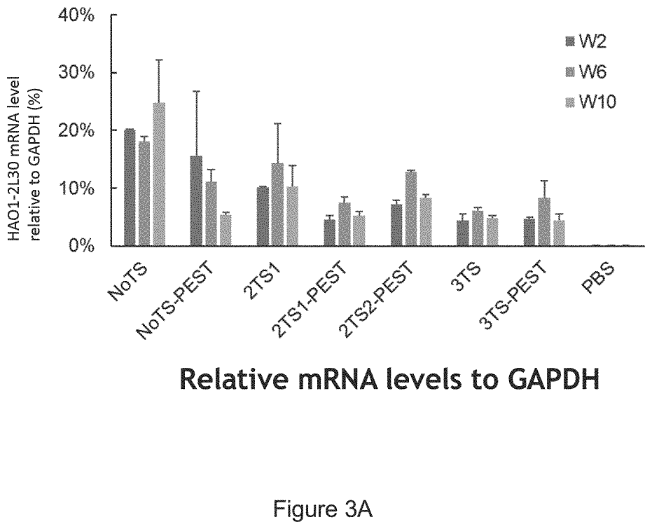

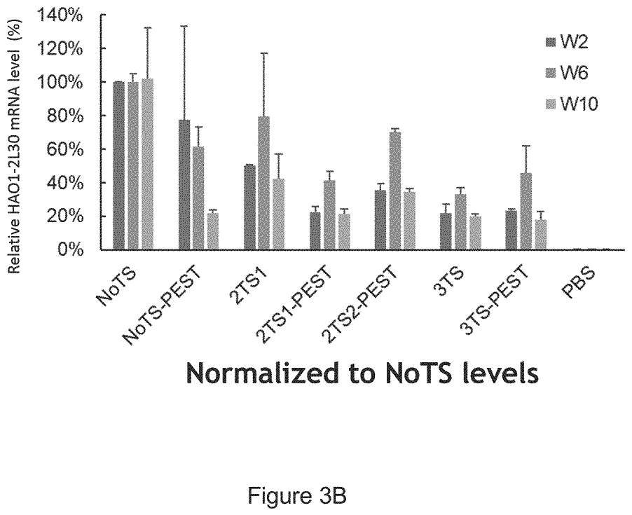

To extract mRNA, small pieces of mouse liver tissues (˜50 mg) were homogenized in TRIzol and mRNA was purified using a TRIzol Plus RNA Purification Kit (Invitrogen, Cat #12183555). An “on-column” DNase purification step was performed using a PureLink DNase Kit (Invitrogen, Cat #12185010). The amount and purity of isolated RNA was determined using a NanoDrop UV-Vis Spectrophotometer (Thermo Scientific) and RNA samples were subsequently diluted to a standard concentration of 100 ng/μL using molecular biology grade water. To generate cDNA, 500 ng of mRNA from each sample was subjected to reverse transcription using iScript Reverse Transcription Supermix (Bio-Rad, Cat #1708841). Using this cDNA as template material, qPCR reactions were prepared in duplicate using KAPA SYBR® FAST qPCR Kit (KAPA Biosystemsm, Cat #KK4602), and run on QuantStudio 7 Flex Real-Time PCR Systems from ThermoFisher. Two primer pairs were used to target either the nuclease ORF (forward 5′ ACTTCTGAAACCGTTCGTGCT 3′ (SEQ ID NO: 3), reverse 5′ GGATGCCTGAGATGGCGATAG 3′ (SEQ ID NO: 4)) or mouse GAPDH gene (forward 5′ TGGTGAAGGTCGGTGTGAAC 3′ (SEQ ID NO: 5), reverse 5′ CCATGTAGTTGAGGTCAATGAAGG 3′ (SEQ ID NO: 6)) as a housekeeping control. Following qPCR, the delta-delta Ct method was employed to determine relative levels of nuclease mRNA. Results from qPCR analysis of HAO 1-2L.30 nuclease mRNA levels is shown in FIGS. 3A and 3B.

Genomic DNA (gDNA) Extraction from Mouse Liver and qPCR

To extract total gDNA, small piece of mouse liver tissues (˜50 mg) was processed using NucleoSpin Tissue, Mini kit (Macherey-Nagel, Cat #740952.50) following manufacture's instruction. Briefly, a small section of liver was placed in a 1.5 ml tube. Lysis was achieved by incubation of the samples in a solution containing SDS and Proteinase K at 65° C. Appropriate conditions for binding of DNA to the silica membrane of the NucleoSpin® Tissue Columns were created by addition of large amounts of chaotropic ions and ethanol to the lysate. The binding process is reversible and specific to nucleic acids. Contaminations were removed by efficient washing with buffer. Pure gDNA was finally eluted under low ionic strength conditions in water. The gDNA concentrations were determined using a NanoDrop UV-Vis Spectrophotometer (Thermo Scientific) and diluted to 60 ng/μL with nuclease-free water. 120 ng total DNA of each mouse was used for qPCR as template to determine AAV copy numbers using three different primer pairs. Because 120 ng mouse genomic DNA is approximate to 21,455 copies of diploid genome given the genome size of FVB/NJ mice at 2.59E+08 base pair (bp), the AAV copy number/diploid genome was calculated by dividing the AAV copy numbers obtained from qPCR by 21455. The qPCR reaction was set up using KAPA SYBR® FAST qPCR Kit (KAPA Biosystemsm, Cat #KK4602), and run on QuantStudio 7 Flex Real-Time PCR Systems from ThermoFisher. SV40 primer pair (forward 5′ GAGTTTGGACAAACCACAACTAGA 3′ (SEQ ID NO: 7) and reverse 5′ AGCAATAGCATCACAAATTTCACAA 3′ (SEQ ID NO: 8)) targets SV40 polyA sequence in AAV genome. TBG primer pair (forward 5′ AAACTGCCAATTCCACTGCTG 3′ (SEQ ID NO: 9) and reverse 5′ CCATAGGCAAAAGCACCAAGA 3′ (SEQ ID NO: 10)) targets TBG promoter in AAV genome. The SV40 polyA sequence and TBG promoter don't contain TS insertion, therefore the AAV copy numbers obtained from qPCR using SV40 and TBG primer pairs represent the total AAV counts in cells. TS1 primer pair (forward 5′ ACAATTCGTGAGGCACTGGG 3′ (SEQ ID NO: 11) and reverse 5′ TGGAGAGAAAGGCAAAGTGGAT 3′ (SEQ ID NO: 12)) flank the region from nuclease start codon to intron in the AAV genome, in which one or two copies of TS were inserted. The AAV copy numbers obtained from qPCR using TS1 primer pairs represent the AAV counts remained after nuclease cleavage of the TS in AAV genome. Therefore, the Indel rates in AAV genome were estimated by the following equation:

AAV Indel rate (%)=100%−(TS1 AAV copy number)/(SV40 AAV copy number).

### Results

Western Blot Results

Images from the western blot analysis are provided in FIG. 2A, and results from quantification of nuclease expression levels are provided in FIG. 2B. By western blot, NoTS group showed highest nuclease levels that remained constant over the time of this experiment. As shown in FIG. 2B, after quantification, the nuclease levels of 2TS1 group and 3TS group were found to be ˜46-59% and ˜17-35% of NoTS group, respectively. When normalized against NoTS group at each time point, the nuclease levels of 2TS1 and 3TS group showed progressive loss over the time of this experiment, suggestive of accelerated nuclease loss as a result of TS insertion in AAV genome. No nuclease protein was detected in all four groups with PEST tag insertion.

qRT-PCR Results

Results from analysis of nuclease mRNA levels by qRT-PCR is provided in FIGS. 3A and 3B. Quantification of mRNA level of nuclease relative to GAPDH is provided in FIG. 3A, and mRNA level of nuclease normalized to NoTS is shown in FIG. 3B. As described in FIGS. 3A and 3B, NoTS group showed highest nuclease mRNA levels that remained constant over the time of this experiment. After quantification, the nuclease levels of 2TS1 group and 3TS group were found to be ˜42-79% and ˜20-33% of NoTS group, respectively, in close agreement with the nuclease protein levels measured by western blot. In addition, the nuclease mRNA was detected in all four groups with PEST tag insertion at 18-78% levels of NoTS group, which clearly demonstrated that nuclease with PEST tag was transcribed into mRNA from AAV genome. The reason that no nuclease protein was detected in all four groups with PEST tag insertion is because PEST tag insertion led to rapid nuclease degradation to the levels undetectable by western blot. Unexpectedly, the NoTS-PEST group showed reduced nuclease mRNA levels compared to NoTS group and the mRNA levels dropped over the time of this experiment. PEST tag insertion should only reduce protein levels, but not mRNA levels.

Absolute AAV Copy Numbers Per Diploid Genome by qPCR

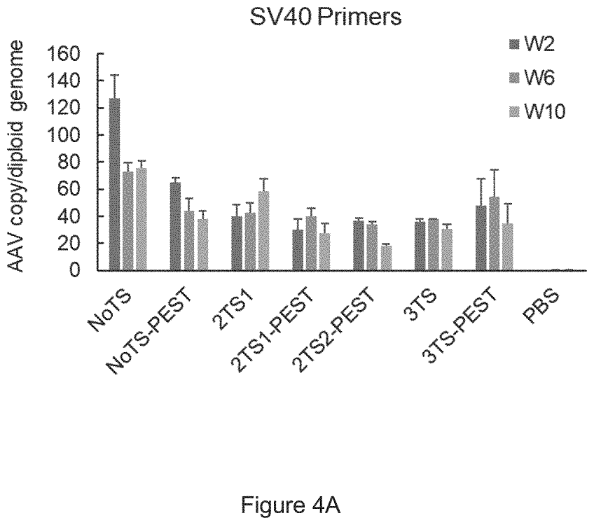

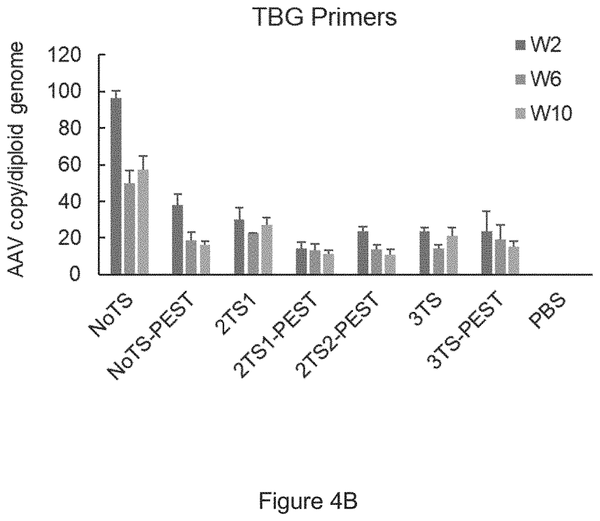

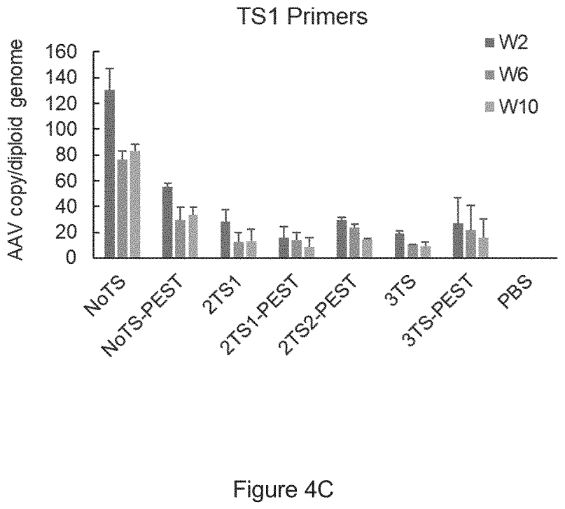

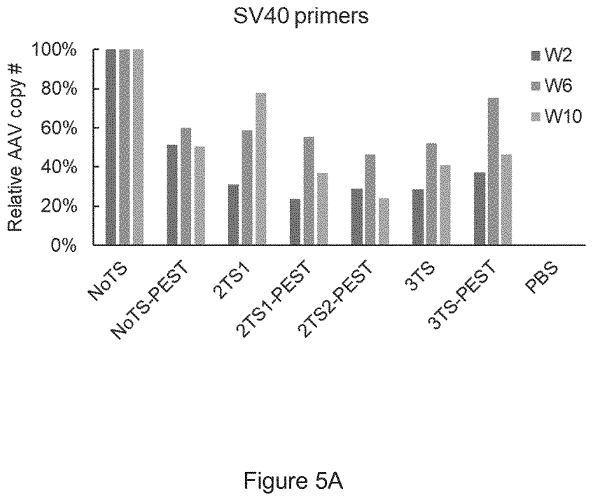

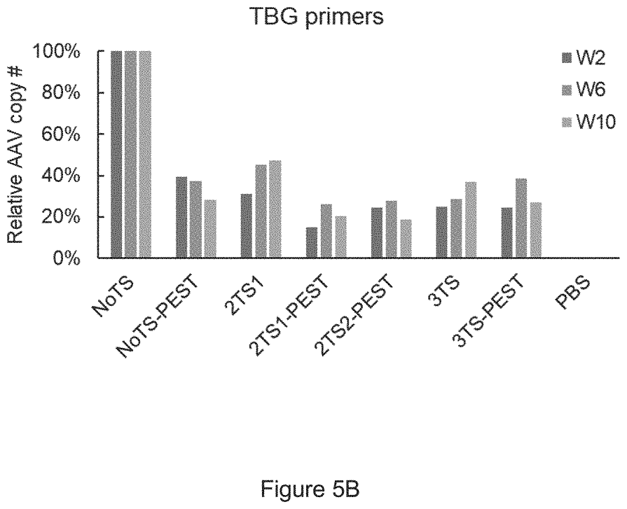

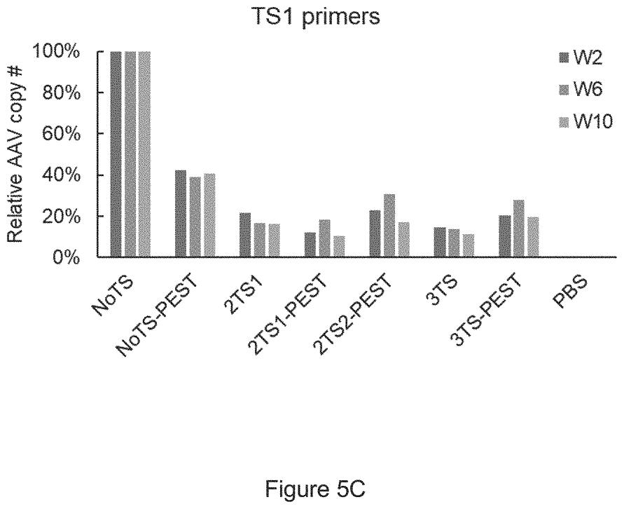

By qPCR, the AAV copy numbers per diploid genome were determined using 3 primer pairs (SV40, TBG and TS1). The results are described in FIGS. 4A-4C and 5A-5C. Quantification of absolute AAV copy number per diploid genome is described in FIGS. 4A-4C. The AAV copy numbers obtained from qPCR using SV40 and TBG primer pairs represented the total AAV counts in cells. The AAV copy numbers obtained from qPCR using TS1 primer pairs represented the AAV counts that remained after nuclease cleavage of the TS in AAV genome because TS1 primer pair flanked a region with TS insertion in the AAV genome. In all five groups with TS insertion, the AAV copy numbers of TS1 primer pair were lower when compared to those of SV40 and TBG primer pairs. This data suggested that nuclease expressed from AAV cut the TS inserted in AAV genome, thus reducing the AAV copy number when measured by TS1 primer pair.

Relative AAV Copy Numbers Per Diploid Genome by qPCR

Quantification of AAV copy number normalized to NoTS levels is described in FIGS. 5A-5C. As described in FIGS. 5A-5C, by normalizing against the AAV copy numbers of NoTS group at each time point, the changes of AAV copy number from the 3 primer pairs became more pronounced. NoTS and NoTS-PEST groups showed similar AAV copy numbers of the 3 primer pairs, which remained constant over the time of this experiment. In all five groups with TS insertion, particularly 2TS1 and 3TS groups, the progressive decrease of AAV copy number over time was only observed in those of TS1 primer pair but not in those of SV40 and TBG primer pairs, which is most likely due to cleavage of TS inserted in AAV genome by nuclease.

### Conclusion

As described in FIGS. 2A-2B, 3A-3B, 4A-4C, and 5A-5C, insertion of TS in the AAV genome led to reduction of nuclease expression on both protein and mRNA levels, and reduction in AAV copy number over time. Addition of a PEST tag to the nuclease led to reduction of nuclease expression on both protein and mRNA levels. In fact, the reduction of nuclease proteins by addition of PEST tag was so dramatic that it was undetectable by western blot analysis.

### Example 2. Determining Insertion and Deletion (Indel) Rates Generated by Nuclease

### Materials and Methods

gDNA Extraction from Mouse Livers

To extract total gDNA, small piece of mouse liver tissues (˜50 mg) was processed using NucleoSpin Tissue, Mini kit (Macherey-Nagel, Cat #740952.50) following manufacture's instruction. Briefly, a small section of liver was placed in a 1.5 ml tube. Lysis was achieved by incubation of the samples in a solution containing SDS and Proteinase K at 65° C. Appropriate conditions for binding of DNA to the silica membrane of the NucleoSpin® Tissue Columns were created by addition of large amounts of chaotropic ions and ethanol to the lysate. The binding process is reversible and specific to nucleic acids. Contaminations are removed by efficient washing with buffer. Pure gDNA is finally eluted under low ionic strength conditions in water. The gDNA concentrations were determined using a NanoDrop UV-Vis Spectrophotometer (Thermo Scientific) and diluted to 15 ng/μL with nuclease-free water.

Insertion and deletion (indel) analysis by digital droplet PCR (ddPCR) gDNA was used for indel quantification using Bio-Rad's QX200 Droplet Digital PCR system. Two taqman assays were multiplexed in the same reaction, one to detect indels at the nuclease target site and a reference assay to act as a housekeeping control in mouse genome. The primer and probe sequences for these assays are shown below:

TABLE A
Primers

| | |
|---|---|
| Target forward primer | ggtgccagaatgtgaaagt |
| | (SEQ ID NO: 13) |
| Target reverse primer | tggtcaccctctgcaca |
| | (SEQ ID NO: 14) |
| Target probe | gacattggtgaggaaaaatcctttgg |
| | (BHQ labelled) |
| | (SEQ ID NO: 15) |
| Reference probe | tgtggtcaccctctgcacagtgt |
| (Mouse) | (REF PROBE1) |
| | (SEQ ID NO: 16) |

Digital droplet PCR (ddPCR) reaction was set up using ddPCR Supermix for Probes (no dUTP) (Catalog #1863024 from Bio-Rad), the target taqman assay (in FAM), the reference taqman assay (in HEX) and HindIII-HF enzyme (NEB Catalog #R3104S) to fragment the genomic DNA. 5000 genome copies of the mock and treated samples were loaded as template in the PCR reaction.

PCR Products for Amplicon-Seq

Q5 High-Fidelity DNA Polymerase (NEB Cat #M0491) was used with the extracted gDNA from each mouse as template to PCR amplify a 336 bp amplicon. Gene specific primers (forward 5′ AGCAGTGAACAGCCAATTGA 3′ (SEQ ID NO: 17), reverse 5′ CCTCTCAAAATGCCCTTTGC 3′ (SEQ ID NO: 18)) were utilized that sat 162 bp upstream and 152 bp downstream of nuclease target site in mouse genome. The PCR products were visualized on a 1% agarose TAE gel and extracted using NucleoSpin® 96 PCR Clean-up kit (Macherey-Nagel Cat #740658.4) as directed by the kit manual.

Next Generation Sequencing (NGS)

Illumina compatible sequencing libraries were generated using NEBNext Ultra DNA Library Prep Kit for Illumina (NEB, Ipswitch, Mass., USA). Paired-end sequencing data was generated for each library using a NextSeq (Illumina, San Diego, Calif., USA). FastQ reads were joined using Flash and aligned with the reference sequence using BWA-MEM. SAM files were analyzed for insertions or deletions occurring within the specified range using a custom script.

### Results

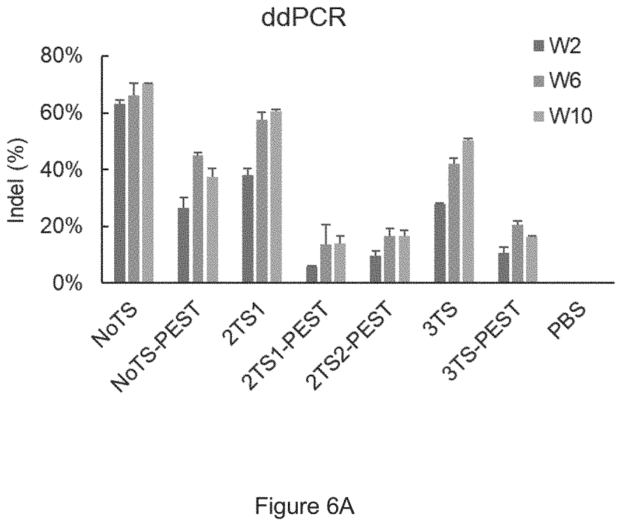

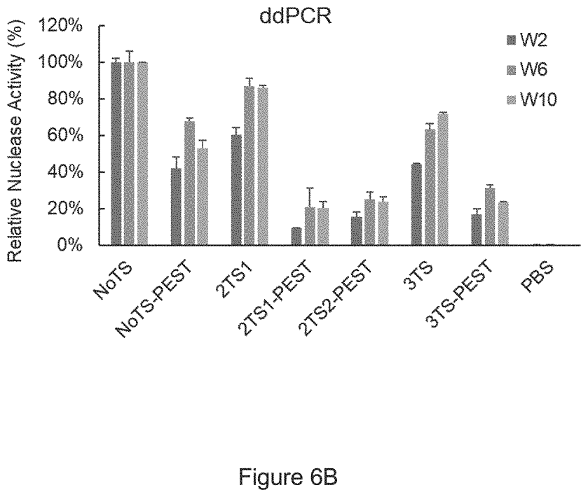

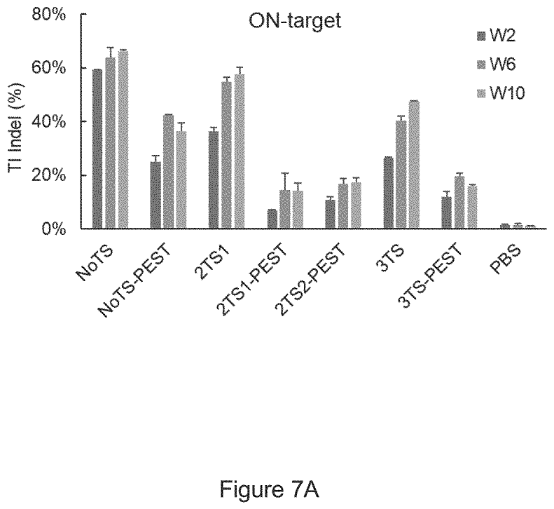

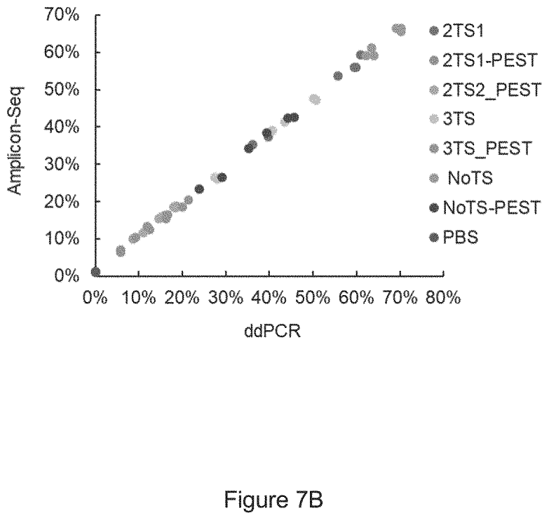

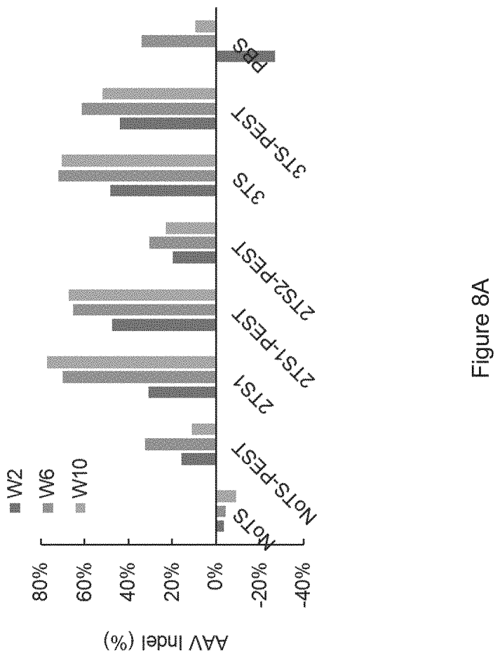

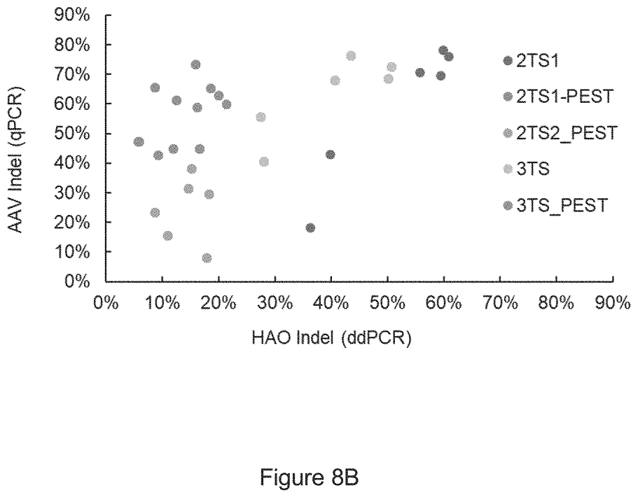

Insertion and deletion (indel) rates generated by nuclease at nuclease target site on mouse genome was measured by ddPCR and Amplicon-seq to determine nuclease activity in mouse liver. The results are described in FIGS. 6A-6B and 7A-7B. Results from experiments directed at determining indel rates in AAV genome are described in FIG. 8A. Correlation between AAV indel rates and genome indel rates (or on-target indel rates) is described in FIG. 8B.

ddPCR Results

Results from experiments directed at measuring indel rates by ddPCR are described in FIGS. 6A-6B. As described in FIGS. 6A-6B, with indel rates ranging from 63-70%, NoTS group showed highest nuclease activity, which remained constant over the time of this experiment. Surprisingly, the indel rates of NoTS-PEST group ranged from 27-45%, equivalent to 42-68% of NoTS group, given the previous data that NoTS-PEST group showed the nuclease levels undetectable by western blot. This unexpected result provided some insight on indel generation in mouse liver that extending nuclease expression is an important factor to introduce the highest level indel in mouse genome. However, even relatively short expression of the nuclease in the case of the PEST containing sequences still led to relatively high levels of indel formation. The indel rates of 2TS1 and 3TS groups increased from 38 to 60% and 28 to 50% over the time of this experiment, equivalent to 60-86% and 44-72% of NoTS group, respectively. Combined with previous western blot results that the nuclease protein levels of the 2TS1 group and 3TS group are ˜46-59% and ˜17-35% of the NoTS group, respectively, this indel result suggested that TS insertion in AAV genome caused less impact on reducing nuclease activity in mouse liver when compared to reducing nuclease levels. The three groups that combined TS and PEST tag insertion showed lowest nuclease activity in mouse liver, with indel rates ranging from 6-17%, indicative of additive impacts on reducing nuclease activity by TS and PEST tag insertion.

Amplicon-Seq Results

In addition to ddPCR, indel rates at nuclease target site in mouse genome were also measured by amplicon-seq. The results are described in FIG. 7A. Interestingly, indel rates determined by amplicon-seq were found to be in close agreement to those determined by ddPCR. As described in FIG. 7B, the similarity between the two data sets was confirmed by a Pearson correlation coefficient at 0.9993 with a P value <0.0001. This provided additional assurance on data validity of nuclease activity in vivo. Therefore, the conclusions reached based on ddPCR results also held true here.

Correlation Between Genome Indel Rates and AAV Indel Rates

For those groups with TS insertion, the indel rates in AAV genome with TS insertion were calculated by the following equation: AAV Indel rate (%)=100%−(TS1 AAV copy number)/(SV40 AAV copy number). The results are described in FIG. 8A. Because the nucleases expressed from AAV should cut its target site in mouse genome and the target sites inserted in AAV genome, a correlation between AAV indel rates (as described in FIG. 8A) and on-target indel rates (as described in FIG. 7A) would be expected. As described in FIG. 8B, the positive correlation between the two data sets was confirmed by a Pearson correlation coefficient at 0.5417 with a P value <0.002.

### Conclusion

As described in FIGS. 6A-6B, 7A-7B, and 8A-8B, TS insertion in AAV genome led to 70-90% on-target indel levels while the nuclease expression was reduced by 40-80%. Furthermore, PEST tag insertion resulted in appreciable indel levels (27-45%) with undetectable nuclease protein levels, suggesting that extended period of nuclease expression is important for indel generation. Hence, TS and PEST tag insertion showed additive effects on reducing nuclease activity, and on-target indel rates were found to be positively correlated with the AAV indel rates as a result of nuclease activity.

### Example 3. Insertion of TS and/or PEST in AAV Genome Increase On-Target Nuclease Activity and Reduce Off-Target Nuclease Activity

### Materials and Methods

gDNA Extraction from Mouse Livers

To extract total gDNA, small piece of mouse liver tissues (˜50 mg) was processed using NucleoSpin Tissue, Mini kit (Macherey-Nagel, Cat #740952.50) following manufacture's instruction. Briefly, a small section of liver was placed in a 1.5 ml tube. Lysis was achieved by incubation of the samples in a solution containing SDS and Proteinase K at 65° C. Appropriate conditions for binding of DNA to the silica membrane of the NucleoSpin® Tissue Columns were created by addition of large amounts of chaotropic ions and ethanol to the lysate. The binding process was reversible and specific to nucleic acids. Contaminations were removed by efficient washing with buffer. Pure gDNA was finally eluted under low ionic strength conditions in water. The gDNA concentrations were determined using a NanoDrop UV-Vis Spectrophotometer (Thermo Scientific) and diluted to 60 ng/μL with nuclease-free water.

PCR Products for Amplicon-Seq

Three amplicons containing different nuclease off-target sites in mouse genome were PCR amplified using Q5 High-Fidelity DNA Polymerase (NEB Cat #M0491) with the extracted gDNA from each mouse as template. Off-target 1 amplicon was 343 bp amplified by primers (forward 5′ AAGCTCTCCAAATACCACAC 3′ (SEQ ID NO: 19), reverse 5′ AACGACACATACATGTATTGCC 3′ (SEQ ID NO: 20)). Off-target 2 amplicon was 415 bp amplified by primers (forward 5′ ACTGTTTGACTTACTGCTGCC 3′ (SEQ ID NO: 21), reverse 5′ TGTATCCTGTGATTGGTCCTG 3′ (SEQ ID NO: 22)). Off-target 4 amplicon was 420 bp amplified by primers (forward 5′ AAGGCTGTTGTCTCCCAGGCAG 3′ (SEQ ID NO: 23), reverse 5′ TTCTGAACTTTGGCTAGCTGG 3′ (SEQ ID NO: 24)). Details of the on-target (ONT) and off-target (OFT) amplicons and primers for those amplicons are provided below in Tables B and C, respectively. The PCR products were visualized on a 1% agarose TAE gel and extracted using NucleoSpin® 96 PCR Clean-up kit (Macherey-Nagel Cat #740658.4) as directed by the kit manual.

TABLE B
On-target and off-target amplicons

| Amplicon | Sequence | Chromosome | BP | Reads | Mismatches |
|---|---|---|---|---|---|
| HAO_ON | AAGACATTGGTGAGGAAAAATC | chr2 | 134324016 | 2001 | |
| | (SEQ ID NO: 25) | | | | |
| HAO_Off1 | TATACATCTGTAAAGAAAAATA | chr10 | 121990375 | 819 | 6 |
| | (SEQ ID NO: 26) | | | | |
| HAO_Of2 | TGGACATGATTAAGGAAACATC | chr11 | 64902548 | 464 | 5 |
| | (SEQ ID NO: 27) | | | | |
| HAO_Off4 | TAGACATTGGTAAAGACAAATA | chr1 | 193578707 | 234 | 4 |
| | (SEQ ID NO: 28) | | | | |

*Mismatches in off-target amplicons are shown in bold

TABLE C
Primers for on-target and off-target amplicons

| | Primer | | | |
|---|---|---|---|---|
| Site | Name | Sequence | Tm | Size |
| OFT 1 | oft1f | AAGCTCTCCAAATACCACAC | 63 | 343 |
| | | (SEQ ID NO: 19) | | |
| | oft1r | AACGACACATACATGTATTGCC | | |
| | | (SEQ ID NO: 20) | | |
| OFT 2 | oft2f | ACTGTTTGACTTACTGCTGCC | 63 | 415 |
| | | (SEQ ID NO: 21) | | |
| | oft2r | TGTATCCTGTGATTGGTCCTG | | |
| | | (SEQ ID NO: 22) | | |
| OFT 4 | oft4f | AAGGCTGTTGTCTCCCAGGCAG | 66 | 420 |
| | | (SEQ ID NO: 23) | | |
| | oft4r | TTCTGAACTTTGGCTAGCTGG | | |
| | | (SEQ ID NO: 24) | | |
| ONT | 28 F2 | AGCAGTGAACAGCCAATTGA | 65 | 336 |
| | | (SEQ ID NO: 17) | | |
| | 27 R2 | CCTCTCAAAATGCCCTTTGC | | |
| | | (SEQ ID NO: 18) | | |

Next Generation Sequencing (NGS)

Illumina compatible sequencing libraries were generated using NEBNext Ultra DNA Library Prep Kit for Illumina (NEB, Ipswitch, Mass., USA). Paired-end sequencing data was generated for each library using a NextSeq (Illumina, San Diego, Calif., USA). FastQ reads were joined using Flash and aligned with the reference sequence using BWA-MEM. SAM files were analyzed for insertions or deletions occurring within the specified range using a custom script.

### Results

Results from Previous Study

A previous cell-based assay identified a list of nuclease off-target sites for the HAO 1-2 L.30 meganuclease in a mouse cell line. The top three off-target sites were confirmed in mouse liver and were chosen to evaluate the off-targeting by self-limiting AAV.

PCR for Amplicon-Seq

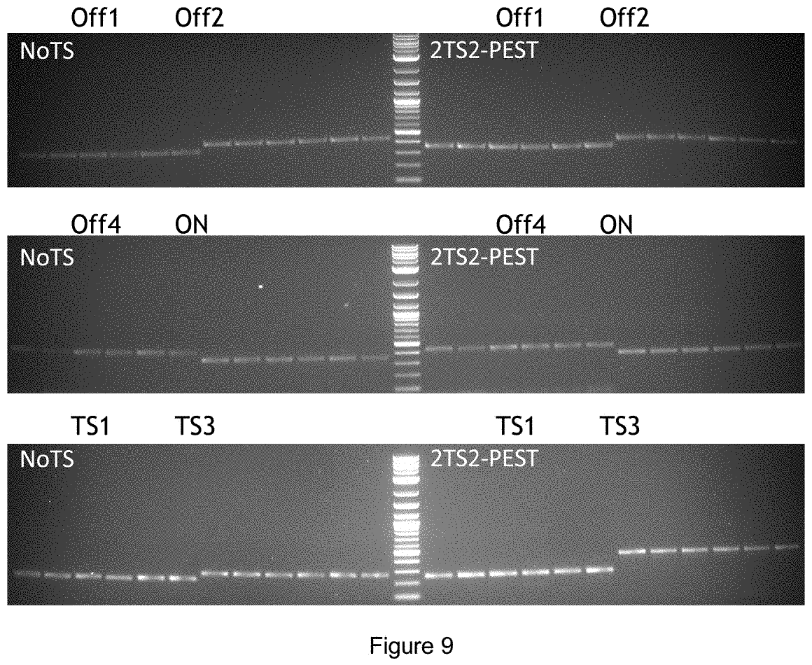

Three amplicons containing top nuclease off-target sites (off-target 1, 2, and 4) in mouse genome were PCR amplified with the extracted gDNA from each mouse as template and were visualized on a 1% agarose TAE gel. The results are described in FIG. 9. The PCR amplicons were purified using NucleoSpin® 96 PCR Clean-up kit (Macherey-Nagel Cat #740658.4) as directed by the kit manual for NGS.

Off-Targeting Nuclease Activity

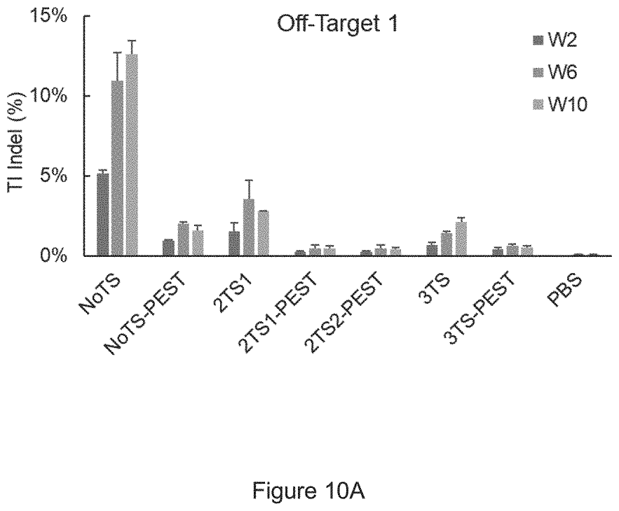

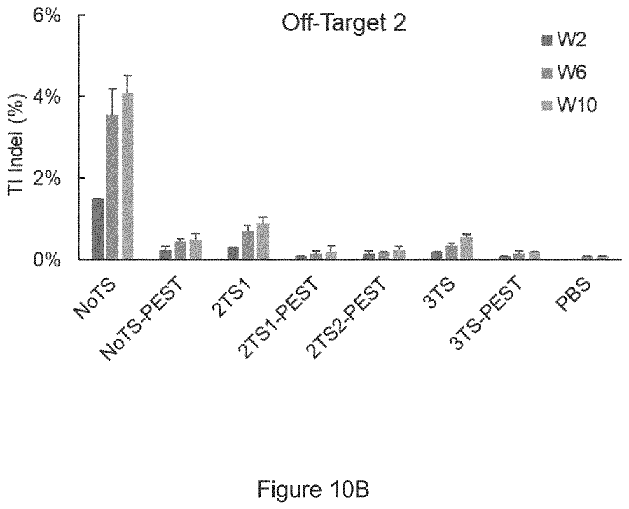

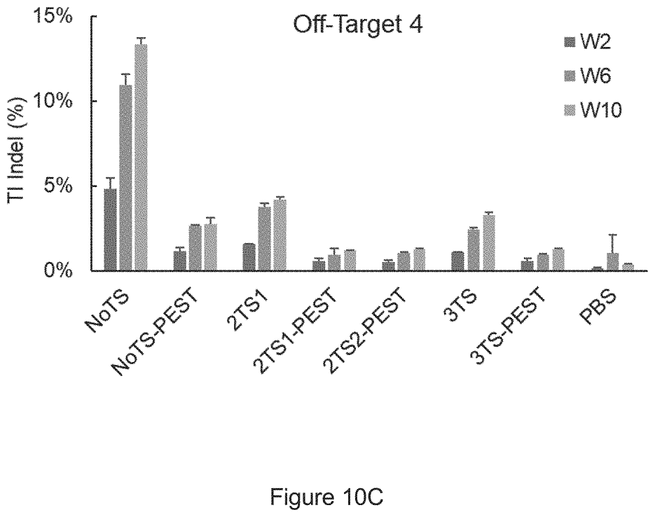

The top three nuclease off-target sites are situated on different mouse chromosomes and were chosen to evaluate the effect of self-inactivating AAV on off-targeting in vivo. Results of experiments directed at determining nuclease activity of self-inactivating AAV on off-target sites are described in FIGS. 10A-10C. As described in FIGS. 10A-10C, NoTS group showed highest nuclease activity across all three off-target sites. The nuclease activity at off-target 1 and off-target 4 was ˜20% of on-target activity (on-target activity is described in FIG. 7A). Therefore, significant off-targeting was observed when using conventional AAV vectors to deliver nuclease. Compared to NoTS group, NoTS-PEST group showed 4-8 fold lower activity across the three off-target sites. This data validated reducing nuclease levels as a functional mechanism to reduce nuclease off-targeting. This reduction of nuclease levels can be achieved by using PEST tag insertion as described here, or through other mechanisms, including a weak promoter to express less nuclease. The off-targeting activity of 2TS1 and 3TS groups were 3-7 fold lower than NoTS group, suggesting that TS insertion is another viable strategy to reduce nuclease off-targeting. The off-targeting activity of three groups that combined TS and PEST tag insertion were further reduced to 8-25 fold lower than NoTS group, which is indicative of additive effects of TS and PEST tag insertion on reducing nuclease activity at off-target sites.

Relative Nuclease Activity at Same Nuclease Protein Level

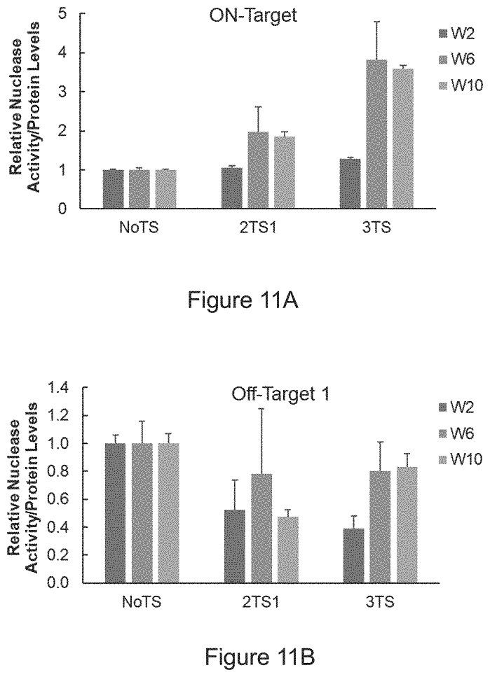

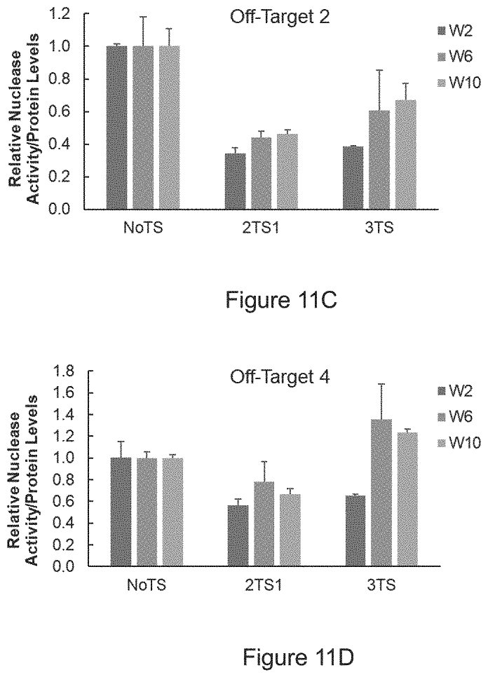

To get a more direct comparison of nuclease activity between groups, the on-target and off-target nuclease activity were normalized against the nuclease protein levels that were determined by western blot (as described in FIG. 2B). The results are described in FIGS. 11A-11D. This analysis revealed relative nuclease activity at the same nuclease protein level of NoTS group. The four groups with PEST insertion were excluded due to undetectable nuclease levels by western blot. As described in FIGS. 11A-11D, when compared to NoTS group, 2TS1 group showed ˜2 fold higher on-target activity and 1.3-3 fold lower off-targeting activity across three off-target sites 6 weeks post AAV administration. The 3TS group showed ˜4 fold higher on-target activity and 1.3-2.5 fold lower off-targeting activity at off-target 1 and off-target 2 at 6 weeks post AAV administration. These results indicated TS insertion as a viable strategy to reduce nuclease off-targeting while maintaining or even enhancing on-target activity.

Relative Nuclease Activity at Same Nuclease mRNA Level

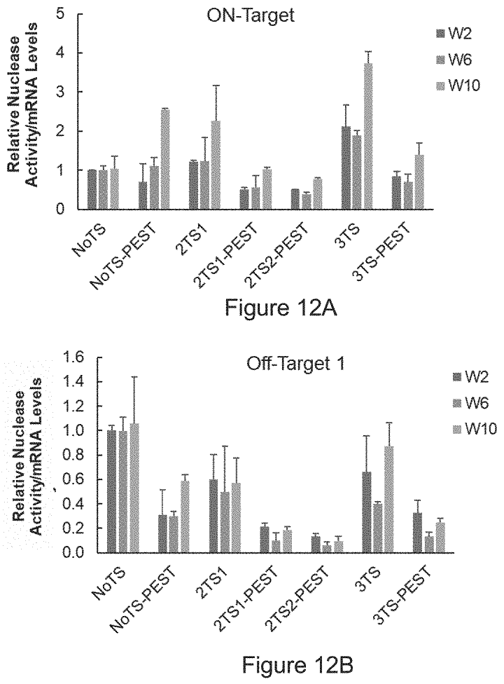

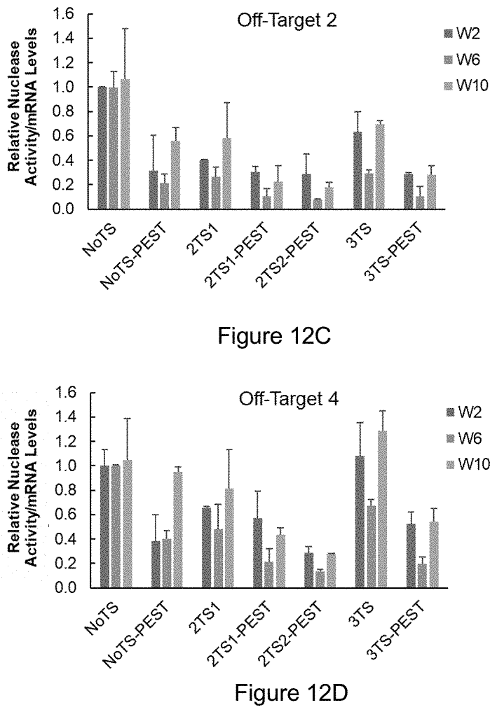

To get a more direct comparison of nuclease activity between all groups, the on-target and off-targeting nuclease activity were normalized against the nuclease mRNA levels determined by qRT-PCR. The results are described in FIGS. 12A-12D. This analysis revealed relative nuclease activity at the same nuclease mRNA level of NoTS group. As described in FIGS. 12A-12D, when compared to NoTS group, NoTS-PEST group showed ˜2 fold higher on-target activity and lower activity across all three off-target sites, validating PEST tag insertion as a viable approach to reduce nuclease off-targeting while maintaining or even enhancing on-target activity. The analysis of 2TS1 and 3TS groups activity on mRNA levels reached similar patterns as those on protein levels (as described in FIGS. 11A-11D), confirming the utility of TS insertion as a viable strategy to reduce nuclease off-targeting while maintaining or enhancing on-target activity. When compared to NoTS group, three groups that combined TS and PEST tag insertion showed similar on-target activity and lower off-targeting activity, likely a result of additive effects of TS and PEST tag insertion on reducing nuclease activity.

### Conclusion

The results described in FIGS. 9, 10A-10C, 11A-11D, and 12A-12D show that both TS insertion and PEST tag insertion are feasible to reduce nuclease activity on off-target sites. These results also indicate that when normalized at the same nuclease protein or mRNA level, both TS insertion and PEST tag insertion are able to increase on-target activity while reducing off-target activity. Furthermore, these results show that combination of TS and PEST tag insertion leads to additive effect on reducing nuclease off-targeting activity.

What is claimed is:

1. A recombinant DNA construct comprising a polynucleotide, wherein said polynucleotide comprises:
(a) a first nucleic acid sequence encoding a first engineered nuclease;
(b) a first promoter operably linked to said first nucleic acid sequence encoding said first engineered nuclease, wherein said promoter is positioned 5′ upstream of said first nucleic acid sequence and drives expression of said first engineered nuclease in a target cell; and
(c) two or more engineered nuclease construct recognition sequences.

2. The recombinant DNA construct of claim 1, wherein said polynucleotide comprises a nuclear localization signal that is positioned 5′ upstream of said first nucleic acid sequence encoding said first engineered nuclease.

3. The recombinant DNA construct of claim 1, wherein said polynucleotide comprises a nuclear localization signal that is positioned 3′ downstream of said first nucleic acid sequence encoding said first engineered nuclease.

4. The recombinant DNA construct of any one of claims 1-3, wherein said polynucleotide comprises an intron that is positioned within said first nucleic acid sequence encoding said first engineered nuclease.

5. The recombinant DNA construct of claim 4, wherein said intron is positioned 3′ downstream of said nuclear localization signal and 5′ upstream of said first nucleic acid sequence encoding said first engineered nuclease.

6. The recombinant DNA construct of claim 4 or 5, wherein at least one of said two or more engineered nuclease construct recognition sequences is positioned 3′ downstream of said intron.

7. The recombinant DNA construct of any one of claims 4-6, wherein at least one of said two or more engineered nuclease construct recognition sequences is positioned 5′ upstream of said intron.

8. The recombinant DNA construct of any one of claims 4-7, wherein at least one of said two or more engineered nuclease construct recognition sequences is positioned within said intron.

9. The recombinant DNA construct of any one of claims 1-8, wherein said first promoter is a tissue-specific promoter, a species-specific promoter, a constitutive promoter or an inducible promoter.

10. The recombinant DNA construct of claim 9, wherein said tissue-specific promoter comprises a liver-specific promoter, an ocular-specific promoter, a central nervous system (CNS)-specific promoter, a lung specific promoter, a skeletal muscle-specific promoter, a heart-specific promoter, or a kidney-specific promoter.

11. The recombinant DNA construct of claim 10, wherein said tissue-specific promoter is a liver-specific promoter.

12. The recombinant DNA construct of claim 11 wherein said liver-specific promoter comprises a human thyroxine binding globulin (TBG) promoter, a human alpha-1 antitrypsin promoter, a hybrid liver specific promoter, or an apolipoprotein A-II promoter.

13. The recombinant DNA construct of claim 10, wherein said tissue-specific promoter is an ocular-specific promoter.

14. The recombinant DNA construct of claim 13, wherein said ocular-specific promoter comprises human G-protein-coupled receptor protein kinase 1 (GRK1) promoter.

15. The recombinant DNA construct of claim 9, wherein said constitutive promoter is a native promoter.

16. The recombinant DNA construct of claim 9, wherein said constitutive promoter is a composite promoter.

17. The recombinant DNA construct of claim 9, wherein said first promoter is an inducible promoter and wherein said polynucleotide further comprises a nucleic acid sequence encoding a ligand-inducible transcription factor, wherein said ligand-inducible transcription factor regulates activation of said first promoter.

18. The recombinant DNA construct of any one of claims 1-17, wherein said polynucleotide further comprises a second nucleic acid sequence encoding a second engineered nuclease.

19. The recombinant DNA construct of claim 18, wherein said first and said second engineered nucleases are different types of nucleases.

20. The recombinant DNA construct of claim 18, wherein said polynucleotide further comprises a second promoter operably linked to said second nucleic acid sequence encoding said second engineered nuclease.

21. The recombinant DNA construct of any one of claims 1-20, wherein said two or more engineered nuclease construct recognition sequences are non-identical.

22. The recombinant DNA construct of any one of claims 1-20, wherein said two or more engineered nuclease construct recognition sequences are identical.

23. The recombinant DNA construct of any one of claims 1-20, wherein said first engineered nuclease binds and cleaves a genomic recognition sequence in a target cell and at least one of said two or more engineered nuclease construct recognition sequences, wherein said genomic recognition sequence is identical to at least one of said two or more engineered nuclease construct recognition sequences.

24. The recombinant DNA construct of claim 23, wherein said first engineered nuclease binds and cleaves a genomic recognition sequence in a target cell and all of said two or more engineered nuclease construct recognition sequences, wherein said genomic recognition sequence is identical to said two or more engineered nuclease construct recognition sequences.

25. The recombinant DNA construct of any one of claims 1-22, wherein said first engineered nuclease binds and cleaves a genomic recognition sequence in a target cell, wherein said first engineered nuclease binds and cleaves at least one of said two or more engineered nuclease construct recognition sequences, wherein said genomic recognition sequence is identical to at least one of said two or more engineered nuclease construct recognition sequences, and wherein one or more second engineered nucleases binds and cleaves at least one of said two or more engineered nuclease construct recognition sequences.

26. The recombinant DNA construct of any one of claims 1-22, wherein said first engineered nuclease binds and cleaves a genomic recognition sequence in a target cell, wherein said genomic recognition sequence is not identical to said two or more engineered nuclease construct recognition sequences.

27. The recombinant DNA construct of claim 26, wherein said first engineered nuclease cleaves at least one of said two or more engineered nuclease construct recognition sequences at about a 50% to about a 90% cleavage rate compared to a cleavage rate of said first engineered nuclease for said genomic recognition sequence.

28. The recombinant DNA construct of claim 26, wherein said first engineered nuclease does not substantially cleave said two or more engineered nuclease construct recognition sequences.

29. The recombinant DNA construct of any one of claims 25-28, wherein a second engineered nuclease binds and cleaves at least one of said two or more engineered nuclease construct recognition sequences.

30. The recombinant DNA construct of claim 29, wherein a second engineered nuclease binds and cleaves all of said engineered nuclease construct recognition sequences.

31. The recombinant DNA construct of claim 29 or claim 30, wherein said second engineered nuclease cleaves said genomic recognition sequence at about 50% to about 90% cleavage rate compared to a cleavage rate of said second engineered nuclease for at least one of said two or more engineered nuclease construct recognition sequences.

32. The recombinant DNA construct of claim 29 or claim 30, wherein said second engineered nuclease does not substantially cleave said genomic recognition sequence.

33. The recombinant DNA construct of any one of claims 26-32, wherein said genomic recognition sequence and at least one of said two or more engineered nuclease construct recognition sequences comprise different center sequences but identical recognition half-site sequences.

34. The recombinant DNA construct of any one of claims 1-33, wherein said recombinant DNA construct further comprises a polyA sequence positioned 3′ downstream of said first nucleic acid sequence encoding said first engineered nuclease.

35. The recombinant DNA construct of any one of claims 1-34, wherein said recombinant DNA construct further comprises a protein degradation peptide encoding sequence positioned 3′ downstream of said first nucleic acid sequence encoding said first engineered nuclease.

36. The recombinant DNA construct of claim 35, wherein said protein degradation peptide comprises a PEST, an intracellular protein degradation signal sequence, a degron sequence, or a ubiquitin sequence.

37. The recombinant DNA construct of claim 35 or 36, wherein said protein degradation peptide encoding sequence is positioned 5′ upstream of at least one of said two or more engineered nuclease construct recognition sequences.

38. The recombinant DNA construct of any one of claims 35-37, wherein said protein degradation peptide encoding sequence is positioned 3′ downstream of at least one of said two or more engineered nuclease construct recognition sequences.

39. The recombinant DNA construct of any one of claims 1-38, wherein said recombinant DNA construct comprises a first engineered nuclease construct recognition sequence and a second engineered nuclease construct recognition sequence.

40. The recombinant DNA construct of claim 39, wherein distance between said first and said second engineered nuclease construct recognition sequences is at least 1000 nucleotides.

41. The recombinant DNA construct of claim 39 or 40, wherein said recombinant DNA construct comprises a polynucleotide, wherein said polynucleotide comprises from 5′ to 3′:
(i) a first promoter sequence, wherein said first promoter sequence is operably linked to a first nucleic acid sequence encoding a first engineered nuclease and drives expression of said first engineered nuclease in a target cell;
(ii) a first engineered nuclease construct recognition sequence positioned 3′ downstream of said first promoter;
(iii) a nuclear localization signal positioned 3′ downstream of said first engineered nuclease construct recognition sequence;
(iv) an intron positioned 3′ downstream of said nuclear localization signal and 5′ upstream of said first nucleic acid sequence encoding said first engineered nuclease;
(v) a second engineered nuclease construct recognition sequence positioned 3′ downstream of said first nucleic acid sequence encoding said first engineered nuclease; and
(vi) a polyA sequence positioned 3′ downstream of said second engineered nuclease construct recognition sequence.

42. The recombinant DNA construct of claim 39 or 40, wherein said recombinant DNA construct comprises a polynucleotide, wherein said polynucleotide comprises from 5′ to 3′:
(i) a first promoter sequence, wherein said first promoter sequence is operably linked to a first nucleic acid sequence encoding a first engineered nuclease and drives expression of said first engineered nuclease in a target cell;
(ii) a first engineered nuclease construct recognition sequence positioned 3′ downstream of said first promoter;
(iii) a nuclear localization signal positioned 3′ downstream of said first engineered nuclease construct recognition sequence;
(iv) an intron positioned 3′ downstream of said nuclear localization signal and 5′ upstream of said first nucleic acid sequence encoding said first engineered nuclease;
(v) a protein degradation peptide encoding sequence positioned 3′ downstream of said first nucleic acid sequence encoding said first engineered nuclease;
(vi) a second engineered nuclease construct recognition sequence positioned 3′ downstream of said protein degradation peptide encoding sequence; and
(vii) a polyA sequence positioned 3′ downstream of said second engineered nuclease construct recognition sequence.

43. The recombinant DNA construct of claim 39 or 40, wherein said recombinant DNA construct comprises a polynucleotide, wherein said polynucleotide comprises from 5′ to 3′:
(i) a first promoter sequence, wherein said first promoter sequence is operably linked to a first nucleic acid sequence encoding a first engineered nuclease and drives expression of said first engineered nuclease in a target cell;
(ii) a nuclear localization signal positioned 3′ downstream of said first promoter;
(iii) an intron positioned 3′ downstream of said nuclear localization signal and 5′ upstream of said first nucleic acid sequence encoding said first engineered nuclease;
(iv) a first engineered nuclease construct recognition sequence positioned within said intron;
(v) a protein degradation peptide encoding sequence positioned 3′ downstream of said first nucleic acid sequence encoding said first engineered nuclease;
(vi) a second engineered nuclease construct recognition sequence positioned 3′ downstream of said protein degradation peptide encoding sequence; and
(vii) a polyA sequence positioned 3′ downstream of said second engineered nuclease construct recognition sequence.

44. The recombinant DNA construct of any one of claims 1-38, wherein said recombinant DNA construct comprises a first engineered nuclease construct recognition sequence, a second engineered nuclease construct recognition sequence, and a third engineered nuclease construct recognition sequence.

45. The recombinant DNA construct of claim 44, wherein distance between said first and said second engineered nuclease construct recognition sequences is at least 50 nucleotides and distance between said second and said engineered nuclease third construct recognition sequences is at least 1000 nucleotides.

46. The recombinant DNA construct of claim 44 or 45, wherein said recombinant DNA construct comprises a polynucleotide, wherein said polynucleotide comprises from 5′ to 3′:
(i) a first promoter sequence, wherein said first promoter sequence is operably linked to a first nucleic acid sequence encoding a first engineered nuclease and drives expression of said first engineered nuclease in a target cell;
(ii) a first engineered nuclease construct recognition sequence positioned 3′ downstream of said first promoter;
(iii) a nuclear localization signal positioned 3′ downstream of said first engineered nuclease construct recognition sequence;
(iv) an intron positioned 3′ downstream of said nuclear localization signal and 5′ upstream of said first nucleic acid sequence encoding said first engineered nuclease;
(v) a second engineered nuclease construct recognition sequence positioned within said intron;
(vi) a third engineered nuclease construct recognition sequence positioned 3′ downstream of said first nucleic acid sequence encoding said first engineered nuclease; and
(vii) a polyA sequence positioned 3′ downstream of said third engineered nuclease construct recognition sequence.

47. The recombinant DNA construct of claim 44 or 45, wherein said recombinant DNA construct comprises a polynucleotide, wherein said polynucleotide comprises from 5′ to 3′:
(i) a first promoter sequence, wherein said first promoter sequence is operably linked to a first nucleic acid sequence encoding a first engineered nuclease and drives expression of said first engineered nuclease in a target cell;
(ii) a first engineered nuclease construct recognition sequence positioned 3′ downstream of said first promoter;
(iii) a nuclear localization signal positioned 3′ downstream of said first engineered nuclease construct recognition sequence;
(iv) an intron positioned 3′ downstream of said nuclear localization signal and 5′ upstream of said first nucleic acid sequence encoding said first engineered nuclease;
(v) a second engineered nuclease construct recognition sequence positioned within said intron;
(vi) a protein degradation peptide encoding sequence positioned 3′ downstream of said first nucleic acid sequence encoding said first engineered nuclease;
(vii) a third engineered nuclease construct recognition sequence positioned 3′ downstream of said protein degradation peptide encoding sequence; and
(viii) a polyA sequence positioned 3′ downstream of said third engineered nuclease construct recognition sequence.

48. The recombinant DNA construct of any one of claims 1-47, wherein said engineered nuclease comprises one or more of an engineered meganuclease, a TALEN, a compact TALEN, a zinc finger nuclease, a CRISPR/Cas9 nuclease, or a megaTAL.

49. The recombinant DNA construct of claim 48, wherein said engineered nuclease comprises an engineered meganuclease.

50. A plasmid comprising the recombinant DNA construct of any one of claims 1-49.

51. A recombinant virus comprising the recombinant DNA construct of any one of claims 1-49.

52. The recombinant virus of claim 51, wherein said recombinant virus is a recombinant adenovirus, a recombinant lentivirus, a recombinant retrovirus, or a recombinant adeno-associated virus (AAV).

53. The recombinant virus of claim 51 or 52, wherein said recombinant virus is a recombinant AAV.

54. The recombinant virus of claim 53, wherein said recombinant AAV has an AAV8 serotype.

55. The recombinant virus of claim 53, wherein said recombinant AAV has an AAV5 serotype.

56. The recombinant virus of claim 53, wherein said recombinant AAV has an AAV2 serotype.

57. A pharmaceutical composition comprising a pharmaceutically acceptable carrier and said plasmid of claim 50.

58. A pharmaceutical composition comprising a pharmaceutically acceptable carrier and said recombinant DNA construct of any one of claims 1-49.

59. A pharmaceutical composition comprising a pharmaceutically acceptable carrier and said recombinant virus of any one of claims 51-56.

60. A method of cleaving a target site in genome of a target cell, said method comprising introducing the plasmid of claim 50 or the recombinant virus of any one of claims 51-56 into the target cell.

61. The method of claim 60, wherein cleavage of said two or more engineered nuclease construct recognition sequences by said engineered nuclease in said target cell increases on-target cleavage of said genome of said target cell by at least 10% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said engineered nuclease.

62. The method of claim 60, wherein cleavage of said two or more engineered nuclease construct recognition sequences by said engineered nuclease in said target cell increases on-target cleavage of said genome of said target cell by about 10-90% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said engineered nuclease.

63. The method of any one of claims 60-62, wherein cleavage of said two or more engineered nuclease construct recognition sequences by said engineered nuclease in said target cell decreases off-target cleavage of said genome of said target cell by at least 10% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said engineered nuclease.

64. The method of any one of claims 60-62, wherein cleavage of said two or more engineered nuclease construct recognition sequences by said engineered nuclease in said target cell decreases off-target cleavage of said genome of said target cell by about 10-90% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said engineered nuclease.

65. The method of any one of claims 60-64, wherein cleavage of said two or more engineered nuclease construct recognition sequences by said engineered nuclease in said target cell reduces the persistence time of said plasmid or said recombinant virus in said target cell when compared to a control plasmid or control recombinant virus that does not comprise two or more engineered nuclease construct recognition sequences cleaved by said engineered nuclease.

66. The method of claim 65, wherein said persistence time in said target cell is less than 10 weeks.

67. The method of claim 66, wherein said persistence time in said target cell is less than 6 weeks.

68. The method of claim 67, wherein said persistence time in said target cell is about 2 weeks.

69. The method of any one of claims 60-68, wherein said engineered nuclease binds and cleaves a genomic recognition sequence in said target cell, and wherein following cleavage of said two or more engineered nuclease construct recognition sequences, integration of said plasmid or said recombinant virus into the genome of said target cell is reduced by at least 10% following at least 2 weeks, at least 6 weeks, or at least 10 weeks after introduction of said plasmid or said recombinant virus into said target cell, when compared to introducing a control plasmid or control recombinant virus that does not comprise two or more engineered nuclease construct recognition sequences cleaved by said engineered nuclease.

70. The method of any one of claims 60-69, wherein said engineered nuclease binds and cleaves a genomic recognition sequence in said target cell, and wherein following cleavage of said two or more engineered nuclease construct recognition sequences, integration of said plasmid or said recombinant virus into the genome of said target cell is reduced by about 10-90% following at least 2 weeks, at least 6 weeks, or at least 10 weeks after introduction of said plasmid or said recombinant virus into said target cell, when compared to introducing a control plasmid or control recombinant virus that does not comprise two or more engineered nuclease construct recognition sequences cleaved by said engineered nuclease.

71. The method of any one of claims 60-70, wherein cleavage of said two or more engineered nuclease construct recognition sequences by said engineered nuclease in said target cell reduces mRNA and/or protein expression of said engineered nuclease in said target cell by at least 10% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said first engineered nuclease.

72. The method of any one of claims 60-71, wherein cleavage of said two or more engineered nuclease construct recognition sequences by said engineered nuclease in said target cell reduces mRNA and/or protein expression of said engineered nuclease in said target cell by about 10-90% following at least 2 weeks, at least 6 weeks, or at least 10 weeks after introduction of said plasmid or said recombinant virus into said target cell, when compared to introduction of a control plasmid or control recombinant virus that does not comprise two or more engineered nuclease construct recognition sequences cleaved by said first engineered nuclease.

73. The method of any one of claims 60-72, wherein cleavage of said two or more engineered nuclease construct recognition sequences by said engineered nuclease in said target cell reduces copy number of said plasmid or said recombinant virus in said target cell following at least 2 weeks, at least 6 weeks, or at least 10 weeks after introduction of said plasmid or said recombinant virus into said target cell by at least 10%, when compared to a control plasmid or control recombinant virus that does not comprise two or more engineered nuclease construct recognition sequences cleaved by said engineered nuclease.

74. The method of any one of claims 60-73, wherein cleavage of said two or more engineered nuclease construct recognition sequences by said engineered nuclease in said target cell reduces copy number of said plasmid or said recombinant virus in said target cell following at least 2 weeks, at least 6 weeks, or at least 10 weeks after introduction of said plasmid or said recombinant virus into said target cell by about 10-90%, when compared to a control plasmid or control recombinant virus that does not comprise two or more engineered nuclease construct recognition sequences cleaved by said engineered nuclease.

75. The method of any one of claims 60-74, wherein cleavage of said two or more engineered nuclease construct recognition sequences by said engineered nuclease in said target cell reduces immunogenic and genotoxic effect of said plasmid or said recombinant virus in said target cell by at least 10% following at least 2 weeks, at least 6 weeks, or at least 10 weeks after introduction of said plasmid or said recombinant virus into said target cell, when compared to a control plasmid or control recombinant virus that does not comprise two or more engineered nuclease construct recognition sequences cleaved by said engineered nuclease.

76. The method of any one of claims 60-75, wherein cleavage of said two or more engineered nuclease construct recognition sequences by said engineered nuclease in said target cell reduces immunogenic and genotoxic effect of said plasmid or said recombinant virus in said target cell by about 10-90% following at least 2 weeks, at least 6 weeks, or at least 10 weeks after introduction of said plasmid or said recombinant virus into said target cell, when compared to a control plasmid or control recombinant virus that does not comprise two or more engineered nuclease construct recognition sequences cleaved by said engineered nuclease.

77. The method of claim 75 or 76, wherein said genotoxic effect comprises translocations, inversions, and/or indels.

78. The method of any one of claims 60-77, wherein the target cell is an eukaryotic cell.

79. The method of claim 78, wherein the eukaryotic cell is a mammalian cell.

80. The method of claim 78 or 79, wherein the eukaryotic cell is a human cell.

81. The method of claim 78, wherein the eukaryotic cell is a plant cell.

82. A method for producing a genetically-modified eukaryotic cell having a disrupted target sequence in a genome of said genetically modified eukaryotic cell, said method comprising:
introducing into said eukaryotic cell the recombinant DNA construct of any one of claims 1-49,
wherein said engineered nuclease is expressed in said eukaryotic cell;
wherein said engineered nuclease produces a cleavage site in said genome at a genomic recognition sequence, and wherein said target sequence is disrupted by non-homologous end-joining at said cleavage site.

83. The method of claim 82, wherein said first engineered nuclease binds and cleaves at least one of said two or more engineered nuclease construct recognition sequences.

84. The method of claim 82, wherein said first engineered nuclease binds and cleaves all of said two or more engineered nuclease construct recognition sequences.

85. The method of any one of claims 82-84, wherein said recombinant DNA construct is introduced into said eukaryotic cell by a recombinant virus.

86. The method of any one of claims 82-85, wherein said eukaryotic cell is a mammalian cell.

87. The method of any one of claims 82-86, wherein said eukaryotic cell is a human cell.

88. The method of any one of claims 82-85, wherein said eukaryotic cell is a plant cell.

89. A method for producing a genetically-modified eukaryotic cell comprising an exogenous sequence of interest inserted into a genome of said eukaryotic cell, said method comprising introducing into said eukaryotic cell one or more recombinant DNA constructs, including:
(a) a recombinant DNA construct of any one of claims 1-49, wherein said engineered nuclease is expressed in said eukaryotic cell; and
(b) a second recombinant DNA construct encoding said sequence of interest;
wherein said engineered nuclease produces a cleavage site in said genome at a genomic recognition sequence; and wherein said sequence of interest is inserted into said genome at said cleavage site.

90. The method of claim 89, wherein said first engineered nuclease binds and cleaves at least one of said two or more engineered nuclease construct recognition sequences.

91. The method of claim 89, wherein said first engineered nuclease binds and cleaves all of said two or more engineered nuclease construct recognition sequences.

92. The method of any one of claims 89-91, wherein said second recombinant DNA construct further comprises sequences homologous to sequences flanking said cleavage site and said sequence of interest is inserted at said cleavage site by homologous recombination.

93. The method of any one of claims 89-92, wherein said recombinant DNA construct is introduced into said eukaryotic cell by a recombinant virus.

94. The method of any one of claims 89-93, wherein said second recombinant DNA construct is introduced into said eukaryotic cell by a recombinant virus.

95. The method of any one of claims 89-94, wherein said eukaryotic cell is a mammalian cell.

96. The method of any one of claims 89-95, wherein said eukaryotic cell is a human cell.

97. The method of any one of claims 89-94, wherein said eukaryotic cell is a plant cell.

98. A genetically-modified eukaryotic cell prepared by the method of any one of claims 82-97.


SEQUENCE LISTING (38 sequences)

SEQ ID NO: 1 - DNA, 24 bp, Artificial Sequence
  Synthetic sequence
  misc_feature (1)..(24): primer (forward) used to determine viral titer by qPCR
gagtttggacaaaccacaactaga

SEQ ID NO: 2 - DNA, 25 bp, Artificial Sequence
  synthetic sequence
  misc_feature (1)..(25): primer (reverse) used to determine viral titer by qPCR
agcaatagcatcacaaatttcacaa

SEQ ID NO: 3 - DNA, 21 bp, Artificial Sequence
  Synthetic Sequence
  misc_feature (1)..(21): primer (forward) used to target nuclease ORF
acttctgaaaccgttcgtgct

SEQ ID NO: 4 - DNA, 21 bp, Artificial Sequence
  Synthetic Sequence
  misc_feature (1)..(21): primer (reverse) used to target nuclease ORF
ggatgcctgagatggcgatag

SEQ ID NO: 5 - DNA, 20 bp, Artificial Sequence
  Synthetic sequence
tggtgaaggtcggtgtgaac

SEQ ID NO: 6 - DNA, 24 bp, Artificial Sequence
  Synthetic Sequence
ccatgtagttgaggtcaatgaagg

SEQ ID NO: 7 - DNA, 24 bp, Artificial Sequence
  Synthetic Sequence
gagtttggacaaaccacaactaga

SEQ ID NO: 8 - DNA, 25 bp, Artificial Sequence
  Synthetic sequence
agcaatagcatcacaaatttcacaa

SEQ ID NO: 9 - DNA, 21 bp, Artificial Sequence
  Synthetic sequence
aaactgccaattccactgctg

SEQ ID NO: 10 - DNA, 21 bp, Artificial Sequence
  Synthetic sequence
ccataggcaaaagcaccaaga

SEQ ID NO: 11 - DNA, 20 bp, Artificial Sequence
  Synthetic sequence
acaattcgtgaggcactggg

SEQ ID NO: 12 - DNA, 22 bp, Artificial Sequence
  Synthetic sequence
tggagagaaaggcaaagtggat

SEQ ID NO: 13 - DNA, 19 bp, Artificial Sequence
  Synthetic sequence
ggtgccagaatgtgaaagt

SEQ ID NO: 14 - DNA, 17 bp, Artificial Sequence
  Synthetic sequence
tggtcaccctctgcaca

SEQ ID NO: 15 - DNA, 26 bp, Artificial Sequence
  Synthetic sequence
gacattggtgaggaaaaatcctttgg

SEQ ID NO: 16 - DNA, 23 bp, Artificial Sequence
  Synthetic sequence
tgtggtcaccctctgcacagtgt

SEQ ID NO: 17 - DNA, 20 bp, Artificial Sequence
  Synthetic sequence
agcagtgaacagccaattga

SEQ ID NO: 18 - DNA, 20 bp, Artificial Sequence
  Synthetic sequence
cctctcaaaatgccctttgc

SEQ ID NO: 19 - DNA, 20 bp, Artificial Sequence
  Synthetic sequence
aagctctccaaataccacac

SEQ ID NO: 20 - DNA, 22 bp, Artificial Sequence
  Synthetic sequence
aacgacacatacatgtattgcc

SEQ ID NO: 21 - DNA, 21 bp, Artificial Sequence
  Synthetic sequence
actgtttgacttactgctgcc

SEQ ID NO: 22 - DNA, 21 bp, Artificial Sequence
  Synthetic sequence
tgtatcctgtgattggtcctg

SEQ ID NO: 23 - DNA, 22 bp, Artificial Sequence
  Synthetic sequence
aaggctgttgtctcccaggcag

SEQ ID NO: 24 - DNA, 21 bp, Artificial Sequence
  Synthetic sequence
ttctgaactttggctagctgg

SEQ ID NO: 25 - DNA, 22 bp, Artificial Sequence
  Synthetic sequence
aagacattggtgaggaaaaatc

SEQ ID NO: 26 - DNA, 22 bp, Artificial Sequence
  Synthetic sequence
tatacatctgtaaagaaaaata

SEQ ID NO: 27 - DNA, 22 bp, Artificial Sequence
  Synthetic sequence
tggacatgattaaggaaacatc

SEQ ID NO: 28 - DNA, 22 bp, Artificial Sequence
  Synthetic sequence
tagacattggtaaagacaaata

SEQ ID NO: 29 - DNA, 2362 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagcctaggcatgcaagccaccatggcaccgaagaagaagcgcaaggtgcatatgaacaccaagtacaacaaggtaagtatcaaggttacaagacaggtttaaggagaccaatagaaactgggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaagctagccgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 30 - DNA, 2488 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagcctaggcatgcaagccaccatggcaccgaagaagaagcgcaaggtgcatatgaacaccaagtacaacaaggtaagtatcaaggttacaagacaggtttaaggagaccaatagaaactgggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccccctaggagtcatggcttcccgccggcggtggcggcgcaggatgatggcacgctgcccatgtcttgtgcccaggagagcgggatggaccgtcaccctgcagcctgtgcttctgctaggatcaatgtgtaagctagccgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 31 - DNA, 2413 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagggtacccaagacattggtgaggaaaaatccctaggcatgcaagccaccatggcaccgaagaagaagcgcaaggtgcatatgaacaccaagtacaacaaggtaagtatcaaggttacaagacaggtttaaggagaccaatagaaactgggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaagctagccaagacattggtgaggaaaaatcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 32 - DNA, 2539 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagggtacccaagacattggtgaggaaaaatccctaggcatgcaagccaccatggcaccgaagaagaagcgcaaggtgcatatgaacaccaagtacaacaaggtaagtatcaaggttacaagacaggtttaaggagaccaatagaaactgggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccccctaggagtcatggcttcccgccggcggtggcggcgcaggatgatggcacgctgcccatgtcttgtgcccaggagagcgggatggaccgtcaccctgcagcctgtgcttctgctaggatcaatgtgtaagctagccaagacattggtgaggaaaaatcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 33 - DNA, 2528 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagcctaggcatgcaagccaccatggcaccgaagaagaagcgcaaggtgcatatgaacaccaagtacaacaaggtaagtatcaaggttacaagacaggtttaaggcgccaatagaataagacattggtgaggaaaaatcggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccccctaggagtcatggcttcccgccggcggtggcggcgcaggatgatggcacgctgcccatgtcttgtgcccaggagagcgggatggaccgtcaccctgcagcctgtgcttctgctaggatcaatgtgtaagctagccaagacattggtgaggaaaaatcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 34 - DNA, 2431 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagggtacccaagacattggtgaggaaaaatccctaggcatgcaagccaccatggcaccgaagaagaagcgcaaggtgcatatgaacaccaagtacaacaaggtaagtatcaaggttacaagacaggtttaaggcgccaatagaataagacattggtgaggaaaaatcggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaagctagccaagacattggtgaggaaaaatcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 35 - DNA, 2557 bp, Artificial Sequence
  Synthetic sequence
ctgcgcgctcgctcgctcactgaggccgcccgggcaaagcccgggcgtcgggcgacctttggtcgcccggcctcagtgagcgagcgagcgcgcagagagggagtggaattcacgcgttgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaagcttaattgtgagcgctcacaattgtgaaccgtcagatcactagaaccttaattgtgagcgctcacaattgtgagactgctgccgcagtcagtaattgtgagcgctcacaattgacctggagctgcaggagttggtaattgtgagcgctcacaattcgtgaggcactgggcagggtacccaagacattggtgaggaaaaatccctaggcatgcaagccaccatggcaccgaagaagaagcgcaaggtgcatatgaacaccaagtacaacaaggtaagtatcaaggttacaagacaggtttaaggcgccaatagaataagacattggtgaggaaaaatcggcttgtcgagacagagaagactcttgcgtttctgataggcacctattggtcttactgacatccactttgcctttctctccacaggagttcttactctacttagcagggtttgtagacggtgacggttccatctatgcagcgatcaggcccagtcagacagctaagttcaagcaccggctgcagctcttttttgcggtctatcaaaagacgcagcgccgttggttcctcgacaagctggtggacgagatcggtgtgggttacgtgactgacgctggcagcgtctccagttactttctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacccgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctttgccagtatccatcctcagcaacgtaataagttcaagcaccagctgtctctccatttcacggtccgtcagaagacacagcgccgttggttccttgacaagctggtggacgagatcggtgtgggttacgtgattgacgagggcagcgtctccagttatcggttaagcaagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccccctaggagtcatggcttcccgccggcggtggcggcgcaggatgatggcacgctgcccatgtcttgtgcccaggagagcgggatggaccgtcaccctgcagcctgtgcttctgctaggatcaatgtgtaagctagccaagacattggtgaggaaaaatcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgggaattgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 36 - DNA, 260 bp, Homo sapiens
gtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccag

SEQ ID NO: 37 - DNA, 346 bp, Polyomavirus SV40
gtatttgcttcttccttaaatcctggtgttgatgcaatgtactgcaaacaatggcctgagtgtgcaaagaaaatgtctgctaactgcatatgcttgctgtgcttactgaggatgaagcatgaaaatagaaaattatacaggaaagatccacttgtgtgggttgattgctactgcttcgattgctttagaatgtggtttggacttgatctttgtgaaggaaccttacttctgtggtgtgacataattggacaaactacctacagagatttaaagctctaaggtaaatataaaatttttaagtgtataatgtgttaaactactgattctaattgtttgtgtattttag

SEQ ID NO: 38 - protein, 354 aa, Artificial Sequence
  Synthetic sequence
MNTKYNKEFLLYLAGFVDGDGSIYAAIRPSQTAKFKHRLQLFFAVYQKTQRRWFLDKLVDEIGVGYVTDAGSVSSYFLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIFASIHPQQRNKFKHQLSLHFTVRQKTQRRWFLDKLVDEIGVGYVIDEGSVSSYRLSKIKPLHNFLTQLQPFLKLKQKQANLVLKIIEQLPSAKESPDKFLEVCTWVDQIAALNDSKTRKTTSETVRAVLDSLSEKKKSSP